(19) United States
(12) Patent Application Publication
(10) Pub. No.: US 2024/0276746 A1
(43) Pub. Date: Aug. 15, 2024

(54) MATERIAL FOR PHOTOELECTRIC CONVERSION ELEMENTS FOR IMAGE PICKUP

(71) Applicant: NIPPON STEEL CHEMICAL & MATERIAL CO., LTD., Tokyo (JP)

(72) Inventors: Munetomo INOUE, Tokyo (JP); Kentaro HAYASHI, Tokyo (JP)

(73) Assignee: NIPPON STEEL CHEMICAL & MATERIAL CO., LTD., Tokyo (JP)

(21) Appl. No.: 18/565,774

(22) PCT Filed: Jul. 13, 2022
(86) PCT No.: PCT/JP2022/027621

§ 371 Date: Nov. 30, 2023

(30) Foreign Application Priority Data
Jul. 15, 2021 (JP) 2021-117404

(51) Int. Cl. H10K 39/32; H10K 30/60; H10K 30/86; H10K 85/20; H10K 85/60; H10K 101/30

(52) U.S. Cl. CPC H10K 39/32; H10K 30/60; H10K 30/86; H10K 85/211; H10K 85/622; H10K 85/633; H10K 85/636; H10K 85/654; H10K 85/6572; H10K 85/6574; H10K 85/6576; H10K 85/658; H10K 2101/30

(57) ABSTRACT

Provided are a material that achieves higher sensitivity and higher resolution of a photoelectric conversion element for imaging, and a photoelectric conversion element using the above material. A material for a photoelectric conversion element for imaging including a carbazole compound represented by (Cz)-L¹-(Cz) and having a structure in which two carbazole rings (Cz) are bonded with L¹, wherein at least one of L¹ and a substituent Ar substituted on the carbazole rings is a group having an aromatic ring structure selected from the following formula (4) or (5). (Here, a ring A is represented by formula (5A); X¹ represents O, S, Se, N—R, or N; and X² represents O, S, or Se.)

### TECHNICAL FIELD

The present invention relates to a material for a photoelectric conversion element and a photoelectric conversion element using the same, and particularly to a material for a photoelectric conversion element useful for an imaging device.

In recent years, development of an organic electronic device using a thin film formed with an organic semiconductor is in progress. Examples thereof include an electroluminescent element, a solar cell, a transistor element, and a photoelectric conversion element. In particular, development of an organic EL element, which is an electroluminescent element with an organic substance, is most advanced among them. The applications for smartphones, TV and the like are in progress, and development for a purpose of further higher functionality is continuously conducted.

On the photoelectric conversion element, an element using a P-N junction of an inorganic semiconductor, such as silicon, has been conventionally developed and practically used, and made are investigations for high functionalization of a digital camera and a camera for a smartphone and investigation for application for a monitoring camera, a sensor for an automobile, and the like. However, problems for these various uses include improving sensitivity and micronizing a pixel (improving resolution). For the photoelectric conversion element using an inorganic semiconductor, a mainly adopted method for obtaining a color image is disposing color filters corresponding to RGB, which are the three primary colors of light, on a light receiving part of the photoelectric conversion element. This method has problems in terms of utilization efficiency of an incident light and resolution, because the method disposes the RGB color filters on a plane (Non Patent Literature 1 and 2).

As a solution for such problems of the photoelectric conversion element, a photoelectric conversion element using an organic semiconductor instead of the inorganic semiconductor is developed (Non Patent Literature 1 and 2). This utilizes an ability to selectively absorb only light having a specific wavelength region with high sensitivity that the organic semiconductor has, and proposed is stacking photoelectric conversion elements composed of organic semiconductors corresponding to the three primary colors of light to solve the problem of improving the sensitivity and improving the resolution. An element in which a photoelectric conversion element composed of the organic semiconductor and a photoelectric conversion element composed of the inorganic semiconductor are stacked is also proposed (Non Patent Literature 3).

Here, the photoelectric conversion element composed of the organic semiconductor is an element having a photoelectric conversion layer composed of a thin film of the organic semiconductor between two electrodes, wherein a hole blocking layer and/or an electron blocking layer is disposed between the photoelectric conversion layer and the two electrodes, as necessary. In the photoelectric conversion element, light having a desired wavelength is absorbed in the photoelectric conversion layer to generate an exciton, and then charge separation of the exciton generates a hole and an electron. Thereafter, the hole and the electron move toward each electrode to convert the light into an electric signal. For a purpose of accelerating this process, a method of applying a bias voltage between both the electrodes is commonly used, but one of objects is reducing a leakage current from both the electrodes generated by applying the bias voltage. Accordingly, it can be mentioned that controlling the move of the hole and the electron in the photoelectric conversion element is a key to exhibit characteristic of the photoelectric conversion element.

The organic semiconductor used for each layer of the photoelectric conversion element can be classified into a P-type organic semiconductor and an N-type organic semiconductor. The P-type organic semiconductor is used as a hole transport material, and the N-type organic semiconductor is used as an electron transport material. To control the move of the hole and the electron in the aforementioned photoelectric conversion element, made are various developments of an organic semiconductor having appropriate physical properties such as hole mobility, electron mobility, an energy value of a highest occupied molecular orbital (HOMO), and an energy value of a lowest unoccupied molecular orbital (LUMO). However, the organic semiconductor still has insufficient characteristics, and has not been utilized in commercial practice.

Patent literature 1 proposes an element using quinacridone as the P-type organic semiconductor and subphthalocyanine chloride as the N-type organic semiconductor for the photoelectric conversion layer, and an indolocarbazole derivative for a first buffer layer disposed between the photoelectric conversion layer and the electrode.

Patent literature 2 proposes an element using, for the photoelectric conversion layer, a chrysenodithiophene derivative as the P-type organic semiconductor and fullerenes or a subphthalocyanine derivative as the N-type organic semiconductor.

Patent literature 3 and 4 proposes an element using a carbazole derivative for the electron blocking layer disposed between the photoelectric conversion layer and the electrode. Patent literature 5 proposes an element using a pyrene derivative or a triphenylene derivative for the electron blocking layer disposed between the photoelectric conversion layer and the electrode. Patent literature 6 proposes an element using a biscarbazole compound and the like for the electron blocking layer.

### CITATION LIST

### Patent Literature

### Patent Literature 1

JP 2018-85427 (A)

### Patent Literature 2

JP 2019-54228 (A)

### Patent Literature 3

JP 2011-228614 (A)

### Patent Literature 4

JP 2021-77888 (A)

### Patent Literature 5

JP 2015-153910 (A)

### Patent Literature 6

JP 2011-176259 (A)

### Non Patent Literature

### Non Patent Literature 1

NHK Science & Technology Research Laboratories R&D No. 132, pp. 4-11 (2012.3)

### Non Patent Literature 2

NHK Science & Technology Research Laboratories R&D No. 174, pp. 4-17 (2019.3)

### Non Patent Literature 3

2019 IEEE International Electron Devices Meeting (IEDM), pp. 16.6.1-16.6.4 (2019)

### SUMMARY OF INVENTION

### Technical Problem

In the use of the photoelectric conversion element for imaging for highly functionalizing a digital camera and a camera for a smartphone and for application for a monitoring camera, a sensor for an automobile, and the like, challenges are further higher sensitivity and higher resolution. In view of such a circumstance, an object of the present invention is to provide a material that achieves higher sensitivity and higher resolution of the photoelectric conversion element for imaging, and a photoelectric conversion element for imaging using the same.

### Solution to Problem

The present inventors have made intensive investigation, and consequently found that using a specific carbazole compound efficiently proceeds a process of generating a hole and an electron by charge separation of an exciton in a photoelectric conversion layer, and a process of moving of the hole and the electron in the photoelectric conversion element. This finding has led to the completion of the present invention.

The present invention is a material for a photoelectric conversion element for imaging, the material comprising a carbazole compound represented by the following general formula (1), (2), or (3).

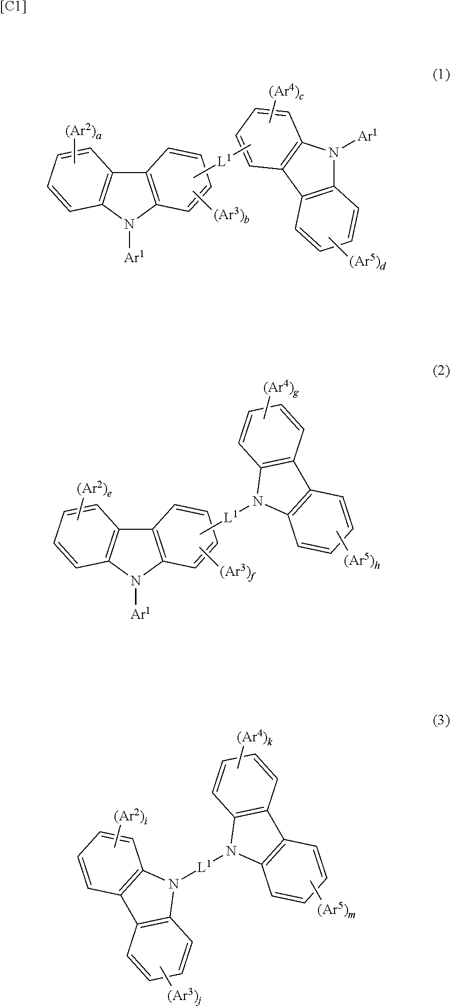

In the formulae (1), (2), and (3), Ar¹ to Ar⁵ each independently represent deuterium, a cyano group, a substituted or unsubstituted diarylamino group having 12 to 30 carbon atoms, a substituted or unsubstituted arylheteroarylamino group having 12 to 30 carbon atoms, a substituted or unsubstituted diheteroarylamino group having 12 to 30 carbon atoms, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to six of aromatic rings of the aromatic hydrocarbon group or the aromatic heterocyclic group are linked; L¹ each independently represents a direct bond, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to six of aromatic rings thereof are linked; “a” to “k” and “m” each independently represent an integer of 0 to 3. At least one of L¹ and Ar¹ to Ar⁵ represents a group having an aromatic ring structure selected from the following formula (4) or (5), and the aromatic ring structure optionally has a substituent. L¹ or Ar¹ to Ar⁵ do not represent an aromatic heterocyclic group having 12 or more carbon atoms except for a case of the group having the aromatic ring structure.

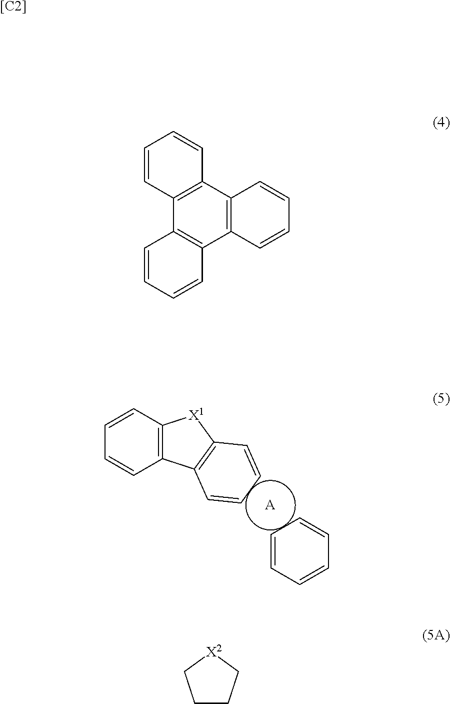

Here, a ring A represents a heterocyclic ring represented by formula (5), and the ring A is fused with an adjacent ring at any position. X¹ represents O, S, Se, N—R, or N, and X² represents O, S, or Se. R represents a substituted or unsubstituted aromatic hydrocarbon group having 6 to 30 carbon atoms, a substituted or unsubstituted aromatic heterocyclic group having 3 to 11 carbon atoms, or a substituted or unsubstituted linked aromatic group in which two to six of aromatic rings thereof are linked.

The Ar¹ to Ar⁵ each independently preferably represent deuterium, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to six of aromatic rings thereof are linked. “a” to “f” each preferably represent 0, and g+h, i+j, and k+m preferably represent 0 or 1.

Among the materials for a photoelectric conversion element represented by the formulae (1) to (3), the material for a photoelectric conversion element represented by formula (1) or (2) is preferable.

The group having the aromatic ring structure is preferably a group represented by the following formula (4a), (4b), (5a), (5b), or (5c):

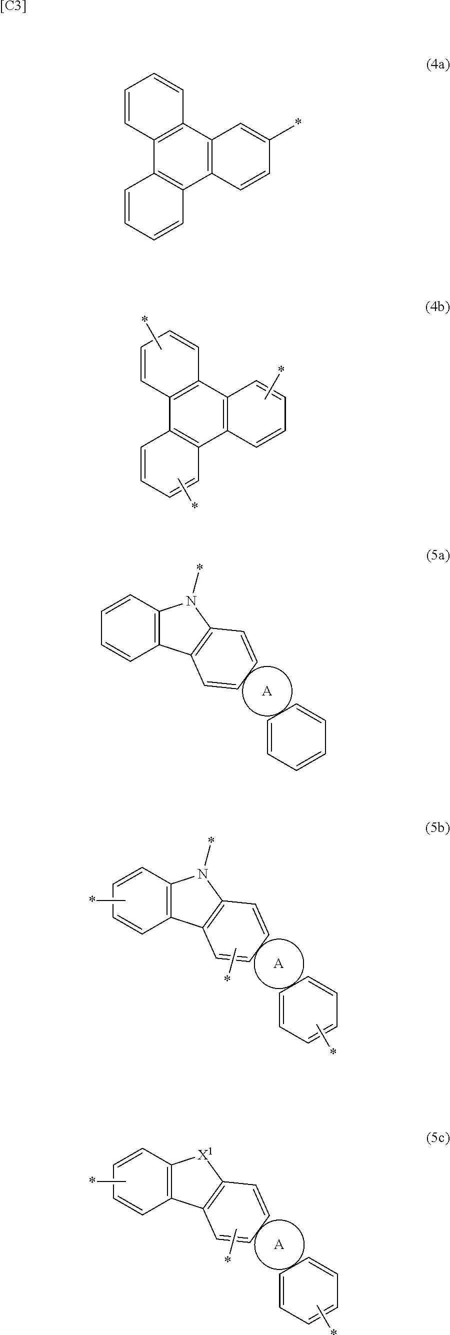

wherein a ring A is same as in formula 5; and “*” represents a bonding point, and at least two thereof are bonding points in formulae (4b), (5b), and (5c).

When the formula (4b) represents a divalent group, the formula (4b) is preferably represented by the following formula (4c).

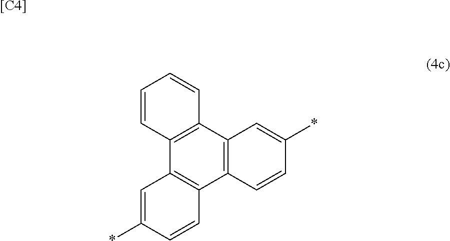

In the material for a photoelectric conversion element, an energy level of highest occupied molecular orbital (HOMO) obtained by structural optimization calculation with a density functional calculation B3LYP/6-31G (d) is preferably −4.5 eV or lower, or an energy level of lowest unoccupied molecular orbital (LUMO) is preferably −2.5 eV or higher.

The material for a photoelectric conversion element preferably has a hole mobility of 1×10⁻⁶ cm²/Vs or more, or is preferably amorphous.

The material for a photoelectric conversion element may be used as a hole transport material.

The present invention is a photoelectric conversion element for imaging, comprising a photoelectric conversion layer and an electron blocking layer between two electrodes, wherein at least one layer of the photoelectric conversion layer or the electron blocking layer contains the above material for a photoelectric conversion element.

In the photoelectric conversion element for imaging of the present invention, the photoelectric conversion layer may contain an electron transport material, and the electron blocking layer may contain the material for a photoelectric conversion element.

### Advantageous Effect of Invention

Using the material for a photoelectric conversion element for imaging of the present invention can achieve appropriate move of the hole and the electron in the photoelectric conversion element, and consequently enables to reduce a leakage current generated by applying a bias voltage during the conversion of light into electric energy. As a result, it is considered that a photoelectric conversion element that achieves a low dark current value and a high contrast ratio can be obtained. The material of the present invention is useful as a material for a photoelectric conversion element for a photoelectric-converting film-stacked imaging device.

### BRIEF DESCRIPTION OF DRAWING

FIG. 1 is a sectional schematic view illustrating a structure example of a photoelectric conversion element for imaging.

### DESCRIPTION OF EMBODIMENTS

The photoelectric conversion element for imaging of the present invention is a photoelectric conversion element having at least one organic layer between two electrodes and converting light into electric energy. This organic layer contains the material for a photoelectric conversion element for imaging represented by any of the general formulae (1) to (3). Hereinafter, the material for a photoelectric conversion element for imaging represented by any of the general formulae (1) to (3) is also referred to as a material for a photoelectric conversion element, a material of the present invention, or the compound represented by the general formulae (1) to (3).

The compound represented by the general formulae (1) to (3) will be described below.

In the general formulae (1) to (3), Ar¹ to Ar⁵ each independently represent deuterium, a cyano group, a substituted or unsubstituted diarylamino group having 12 to 30 carbon atoms, a substituted or unsubstituted arylheteroarylamino group having 12 to 30 carbon atoms, a substituted or unsubstituted diheteroarylamino group having 12 to 30 carbon atoms, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to six of aromatic rings of the aromatic hydrocarbon group or the aromatic heterocyclic group are linked.

L¹ each independently represents a direct bond, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to six of aromatic rings thereof are linked.

At least one of L¹ and Ar¹ to Ar⁵ represents a group having the aromatic ring structure represented by the formula (4) or (5), and the aromatic ring structure optionally has a substituent. L¹ or Ar¹ to Ar⁵ do not represent an aromatic heterocyclic group having 12 or more carbon atoms except for a case of the group having the aromatic ring structure. In a case of the linked aromatic group having the aromatic heterocyclic group, the linked aromatic group does not have an aromatic heterocyclic group having 12 or more carbon atoms.

Specific examples of the case where Ar¹ to Ar⁵ represent an unsubstituted diarylamino group having 12 to 30 carbon atoms, an unsubstituted arylheteroarylamino group having 12 to 30 carbon atoms, or an unsubstituted diheteroarylamino group having 12 to 30 carbon atoms include diphenylamino, dibiphenylamino, phenylbiphenylamino, naphthylphenylamino, dinaphthylamino, triphenylenephenylamino, dianthranilamino, diphenanthrenylamino, dibenzofuranylphenylamino, dibenzofuranylbiphenylamino, phenylcarbazolephenylamino, or bisdibenzofuranylamino. Preferable examples thereof include diphenylamino, dibiphenylamino, phenylbiphenylamino, naphthylphenylamino, dinaphthylamino, triphenylenephenylamino, dibenzofuranylphenylamino, dibenzofuranylbiphenylamino, and bisdibenzofuranylamino. More preferable examples thereof include diphenylamino, phenylbiphenylamino, triphenylenephenylamino, dibenzofuranylphenylamino, or dibenzofuranylbiphenylamino. The aryl group constituting the above amino groups is preferably an aryl group having 6 to 18 carbon atoms, and the heteroaryl group is preferably a heteroaryl group having 6 to 15 carbon atoms. A number of carbon atoms of these amino groups is preferably 12 to 24. A heteroatom in the heteroaryl group is preferably N, S, or O.

L¹ and Ar¹ to Ar⁵ may represent a substituted or unsubstituted aromatic hydrocarbon group, a substituted or unsubstituted aromatic heterocyclic group, or a substituted or unsubstituted linked aromatic group in which two to six of aromatic rings thereof are linked. Although L¹ represents a divalent group and Ar¹ to Ar⁵ represent a monovalent group, these are groups obtained by removing one or two hydrogens from a corresponding aromatic hydrocarbon compound, aromatic heterocyclic compound, or linked aromatic compound. Thus, L¹ and Ar¹ to Ar⁵ will be collectively described.

When L¹ or Ar¹ to Ar⁵ represent the unsubstituted aromatic hydrocarbon group having 6 to 30 carbon atoms, the aromatic hydrocarbon group is a group obtained by removing one or two hydrogens from an aromatic hydrocarbon. Examples of the aromatic hydrocarbon include: monocyclic aromatic hydrocarbons, such as benzene; bicyclic aromatic hydrocarbons, such as naphthalene; tricyclic aromatic hydrocarbons, such as indacene, biphenylene, phenalene, anthracene, phenanthrene, and fluorene; tetracyclic aromatic hydrocarbons, such as fluoranthene, acephenanthrylene, aceanthrylene, triphenylene, pyrene, chrysene, tetraphene, tetracene, and pleiadene; and pentacyclic aromatic hydrocarbons, such as picene, perylene, pentaphene, pentacene, tetraphenylene, and naphthoanthracene. The aromatic hydrocarbon group is preferably benzene, naphthalene, anthracene, triphenylene, or pyrene.

When L¹ or Ar¹ to Ar⁵ represent the unsubstituted aromatic heterocyclic group having 3 to 18 carbon atoms, the aromatic heterocyclic group is a group obtained by removing one or two hydrogens from an aromatic heterocyclic compound. Examples of the aromatic heterocyclic compound include: nitrogen-containing aromatic compounds having a pyrrole ring, such as pyrrole, pyrrolopyrrole, indole, isoindole, pyrroloisoindole, and carboline; thiophene, benzothiophene, furan, benzofuran, pyridine, pyrimidine, triazine, quinoline, isoquinoline, quinazoline, or quinoxaline. The aromatic heterocyclic compound is preferably thiophene, benzothiophene, furan, benzofuran, pyridine, pyrimidine, triazine, quinoline, isoquinoline, quinazoline, or quinoxaline. Examples thereof also preferably include an unsubstituted nitrogen-containing aromatic compound having the structure represented by formula (5).

The group having the aromatic ring structure represented by formula (4) or formula (5) optionally has a substituent, or may be contained as one aromatic group constituting a linked aromatic group. Preferable examples of the group having the structure represented by formula (4) include a triphenylene group. Preferable examples of the group having the structure represented by formula (5) include a group in which X¹ represents N.

The linked aromatic group herein refers to an aromatic group in which aromatic rings of two or more aromatic groups are bonded and linked with a single bond. These linked aromatic groups may be linear or branched. A linking position in linking the benzene rings each other may be any of ortho, meta, and para. The aromatic group may be an aromatic hydrocarbon group or an aromatic heterocyclic group. The plurality of the aromatic groups may be same as or different from each other.

The aromatic ring constituting the linked aromatic group is an aromatic ring in the aromatic hydrocarbon group or the aromatic heterocyclic group, and these aromatic rings are bonded with a single bond. A number of the bonded aromatic rings is two to six, and preferably two to five. Preferable examples of the aromatic ring in the aromatic hydrocarbon group or the aromatic heterocyclic group include benzene, naphthalene, anthracene, triphenylene, pyrene, thiophene, benzothiophene, furan, benzofuran, pyridine, pyrimidine, triazine, quinoline, isoquinoline, quinazoline, quinoxaline ring, or an aromatic ring represented by formula (4) or (5). The aromatic ring is more preferably benzene, naphthalene, anthracene, triphenylene, quinoline, or quinazoline ring.

The diarylamino group, the arylheteroarylamino group, the diheteroarylamino group, the aromatic hydrocarbon group, the aromatic heterocyclic group, or the linked aromatic group optionally has a substituent. Examples of the substituent include deuterium, a cyano group, a diarylamino group having 12 to 30 carbon atoms, an arylheteroarylamino group having 12 to 30 carbon atoms, a diheteroarylamino group having 12 to 30 carbon atoms, and an alkyl group having 1 to 20 carbon atoms. Specific examples of the case of the diarylamino group, the arylheteroarylamino group, or the diheteroarylamino group refer to the case where Ar¹ to Ar⁵ represent these groups.

The alkyl group having 1 to 20 carbon atoms may be any of linear, branched, and cyclic alkyl groups, and is preferably a linear, branched, or cyclic alkyl group having 1 to 10 carbon atoms. Specific examples thereof include: linear saturated hydrocarbon groups, such as a methyl group, an ethyl group, a n-propyl group, a n-butyl group, a n-pentyl group, a n-hexyl group, a n-octyl group, a n-dodecyl group, a n-tetradecyl group, and a n-octadecyl group; branched saturated hydrocarbon groups, such as an isopropyl group, an isobutyl group, a neopentyl group, a 2-ethylhexyl group, and a 2-hexyloctyl group; and saturated alicyclic hydrocarbon groups, such as a cyclopentyl group, a cyclohexyl group, a cyclooctyl group, a 4-butylcyclohexyl group, and a 4-dodecylcyclohexyl group.

Preferable aspects of the general formula (1) include the following formulae (1a) to (1f), and the formulae (1d) to (1f) are more preferable. Here, reference signs common with the general formula (1) have the same means.

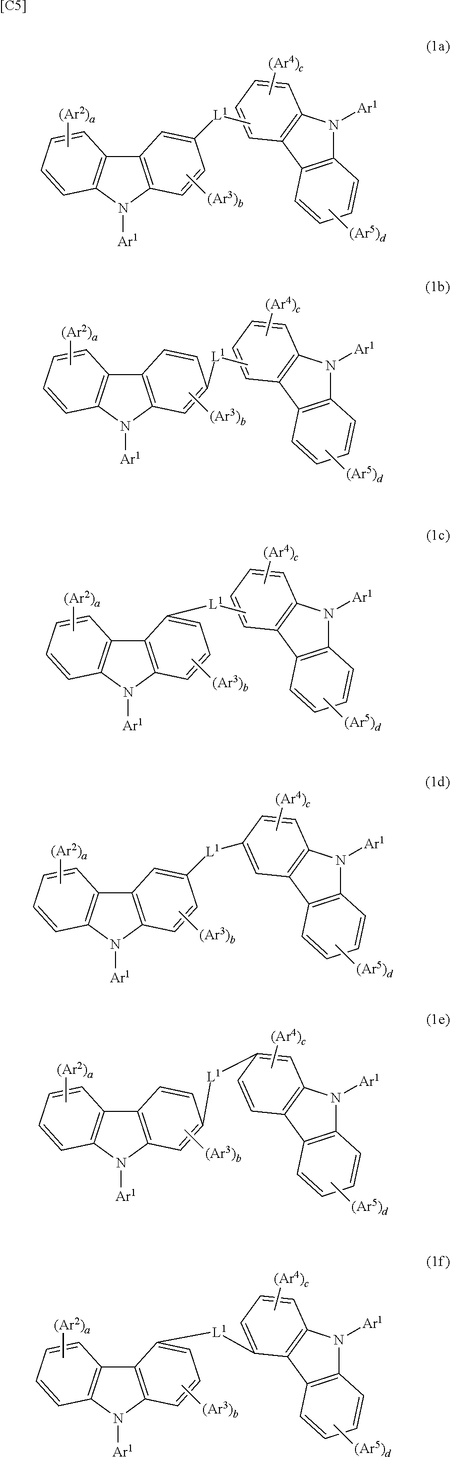

Preferable aspects of the general formula (2) include the following formulae (2a) to (2c), and the formulae (2a) and (2c) are more preferable. Here, reference signs common with the general formula (2) have the same means.

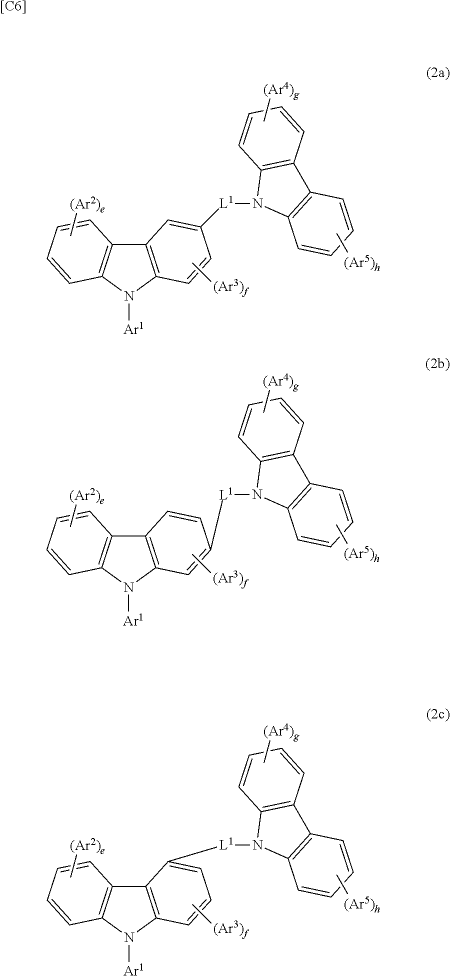

When at least one of L¹ and Ar¹ to Ar⁵ has the aromatic ring structure selected from the formula (4), at least one of L¹ and Ar² to Ar⁵ preferably has the aromatic ring structure of formula (4), and at least one of L¹, Ar⁴, and Ar⁵ more preferably has the aromatic ring structure of formula (4). When at least one of L¹ and Ar¹ to Ar⁵ has the aromatic ring structure of formula (5), at least one of L¹ and Ar² to Ar⁵ preferably has the aromatic ring structure of formula (5), and at least one of Ar⁴ and Ar⁵ more preferably has the aromatic ring structure of formula (5).

In formula (5), a ring A represents a heterocyclic ring represented by formula (5A), and the ring A is fused with an adjacent ring at any position.

X¹ represents O, S, Se, N—R, or N, and X² represents O, S, or Se. X¹ preferably represents N—R or N, and X² preferably represents O or S. In a case of N, the N may be bonded to the carbazole ring at the N-position.

R represents a substituted or unsubstituted aromatic hydrocarbon group having 6 to 30 carbon atoms, a substituted or unsubstituted aromatic heterocyclic group having 3 to 11 carbon atoms, or a substituted or unsubstituted linked aromatic group in which two to six of aromatic rings thereof are linked.

Examples of the unsubstituted aromatic hydrocarbon group having 6 to 30 carbon atoms include groups obtained by removing one hydrogen from an aromatic hydrocarbon. Examples of the aromatic hydrocarbon include: monocyclic aromatic hydrocarbons, such as benzene; bicyclic aromatic hydrocarbons, such as naphthalene; tricyclic aromatic hydrocarbons, such as indacene, biphenylene, phenalene, anthracene, phenanthrene, and fluorene; tetracyclic aromatic hydrocarbons, such as fluoranthene, acephenanthrylene, aceanthrylene, triphenylene, pyrene, chrysene, tetraphene, tetracene, and pleiadene; and pentacyclic aromatic hydrocarbons, such as picene, perylene, pentaphene, pentacene, tetraphenylene, and naphthoanthracene. The aromatic hydrocarbon group is preferably benzene, naphthalene, anthracene, triphenylene, or pyrene.

As the unsubstituted aromatic heterocyclic group having 3 to 11 carbon atoms, groups obtained by removing one or two hydrogens from an aromatic heterocyclic compound may be used. Examples of the aromatic heterocyclic compound include nitrogen-containing aromatic compounds having a pyrrole ring, such as pyrrole, pyrrolopyrrole, indole, pyrroloindole, benzoindole, naphthopyrrole, isoindole, pyrroloisoindole, benzoisoindole, naphthoisopyrrole, and carboline. Preferable examples thereof include thiophene, benzothiophene, furan, benzofuran, pyridine, pyrimidine, triazine, quinoline, isoquinoline, quinazoline, or quinoxaline.

When R represents the aromatic hydrocarbon group or the aromatic heterocyclic group, R optionally has a substituent. Examples of the substituent are same as the substituent when the L¹ or Ar¹ to Ar⁵ represent these groups.

The aromatic ring structure of formula (4) or formula (5) has one or more bonding points (or bonds; represented by “*”). When the aromatic ring structure has a substituent or is contained as a constitutional unit of the linked aromatic group, the aromatic ring structure has a plurality of bonds. In the other cases and a case of L¹, the aromatic ring structure has two bonds, and in the other cases and a case of Ar¹ to Ar⁵, the aromatic ring structure has one bond. The aromatic ring structure may be bonded at any position.

The group having the aromatic ring structure is preferably the group represented by formula (4a), (4b), (4c), (5a), (5b), or (5c). Here, the formulae (4a) and (5a) represent a monovalent group, and a case where Ar¹ to Ar⁵ have one bond corresponds thereto. The formulae (4b), (5b), and (5c) represent divalent or more group, and in a case of the divalent group, a case where L¹ has two bonds corresponds thereto. The formula (4c) represents a divalent group, and it can be mentioned that the formula (4c) is a preferable aspect where the formula (4b) represents a divalent group. When the group has a substituent, the group is bonded to the substituent preferably at a bonding point represented by the formula (4a), (4b), (5a), (5b), or (5c), but may be bonded at other bonding points. When the group has the aromatic ring structure as a constituent of the linked aromatic group, the group is preferably linked at the above bonding point or preferably bonded to the carbazolyl group at the end.

The formula (4a) is a case where the group having the formula (4) as the aromatic ring structure is a monovalent group, and the formula (4b) is a case where the group is a divalent or more group. The formula (5a) is a case where the group having the formula (5) as the aromatic ring structure is a monovalent group, and the formula (5a) or (5c) is a case where the group is a divalent or more group. Although “*” in the formula (4b), (5b), or (5c) represents a bonding point, in a case where a number of the bonds is two or less, the other “*” represent hydrogens (or bonding points to a substituent).

When the group is represented by the formula (4b), the same benzene ring optionally has a plurality of bonding points.

In a case of the group having the aromatic ring structure of formula (4), the group is preferably represented by any of the following formulae (4a) and (4c) to (4g). The formula (4a) or (4c) is more preferable. “*” represents a bonding point.

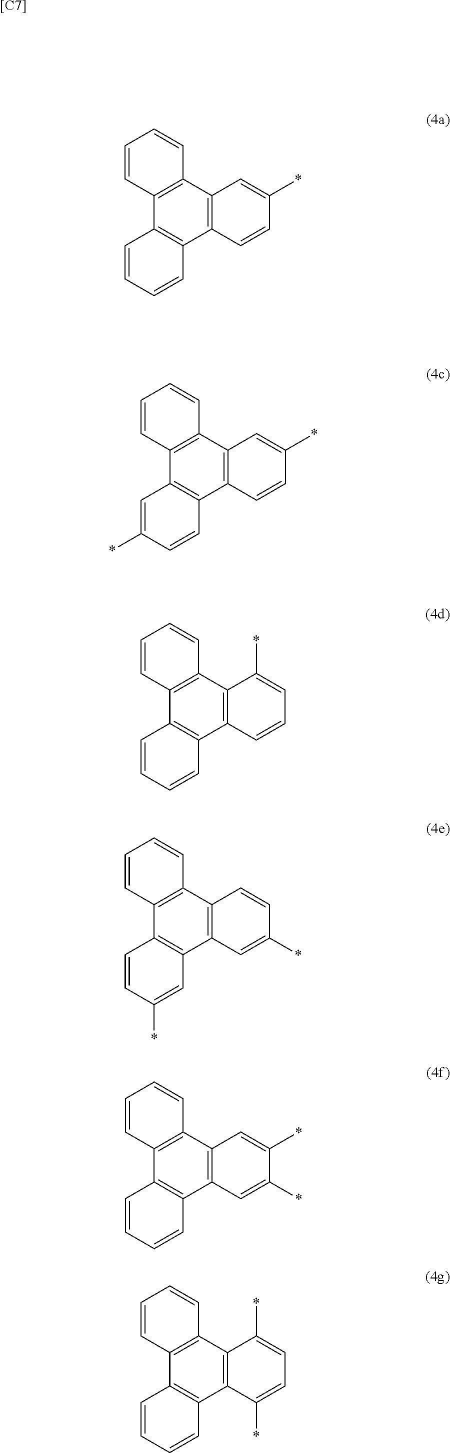

In a case of the group having the aromatic ring structure of formula (5), the ring A may be bonded at any position, but the structure represented by formula (5a), (5b), or (5c) is preferable. The ring A is represented by the formula (5A), and X² represents O, S, or Se. The formula (5a) represents a monovalent group and the formula (5b) or (5c) represents a divalent or more group, but the divalent or more group is preferably the formula (5b). The formula (5b) is a case where X¹ in the formula (5) represents N. In the formula (5c), in a case where X¹ represents N—R, the group may be bonded on R.

In a case of the group having the aromatic ring structure of formula (5), the structure represented by the following formulae (5a) and (5d) to (5j), or bonding at the bonding point represented by “*” or bonding on R when X¹ represents N—R is preferable. The formula (5a) is more preferable.

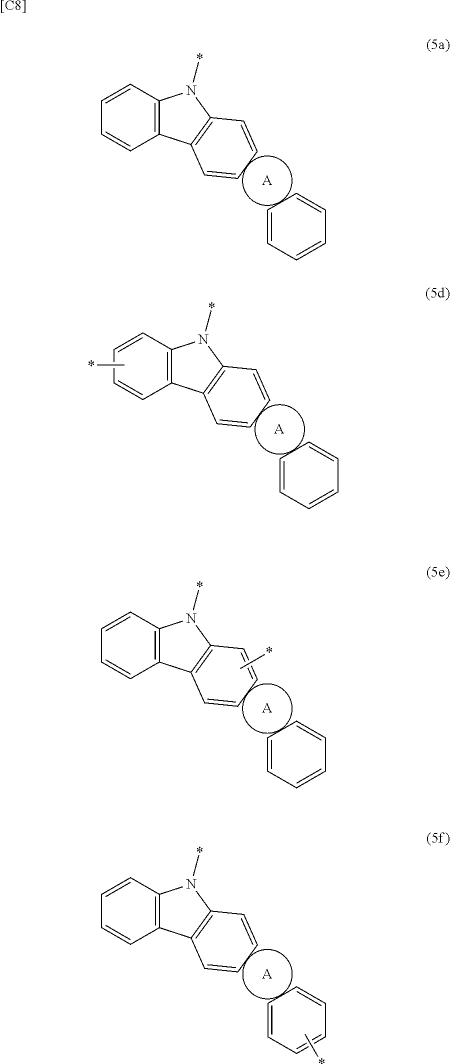

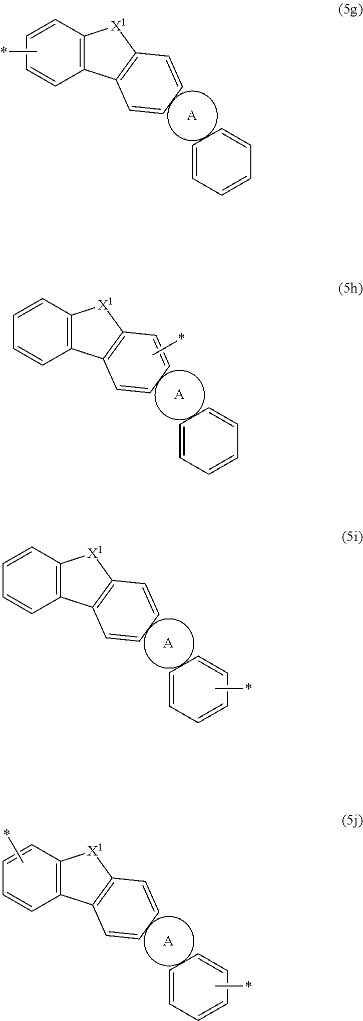

In a case of the group having the aromatic ring structure of formula (5) and there are two or more bonding points, the structure may have a plurality of bonding points in the same benzene ring.

The aromatic ring structure of formula (5) is represented by the following formulae (5k) to (5g), preferably represented by the formula (5k), formula (5l), formula (5n), formula (5p), or formula (5q), and more preferably represented by the formula (5k), formula (5n), or formula (5g).

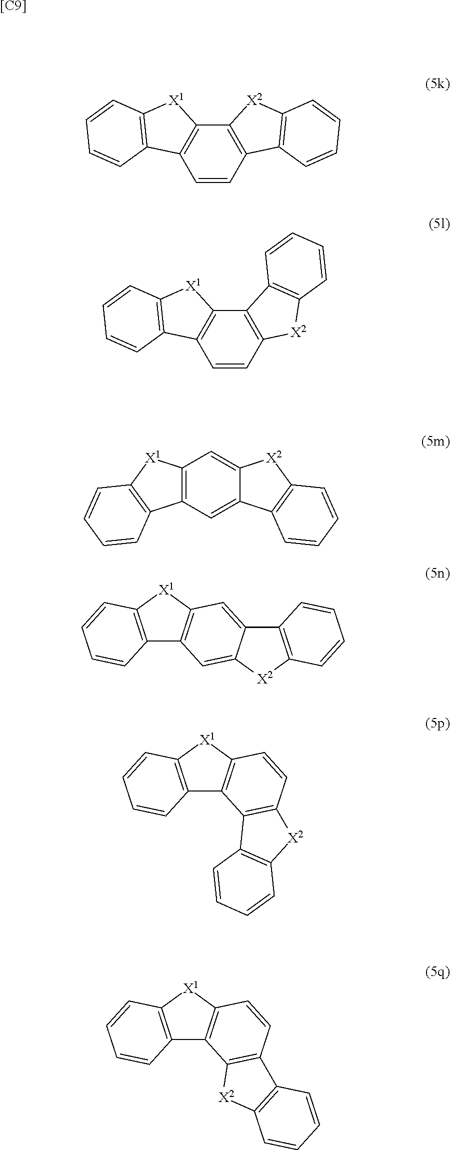

In the general formulae (1) to (3), “a” to “m” each independently represent an integer of 0 to 3. “a” to “f” preferably represent 0, and g+h, i+j, and k+m preferably represent 0 or 1.

Preferable specific examples of the compound represented by the general formula (1) or the material for a photoelectric conversion element of the present invention will be shown below, but are not limited thereto.

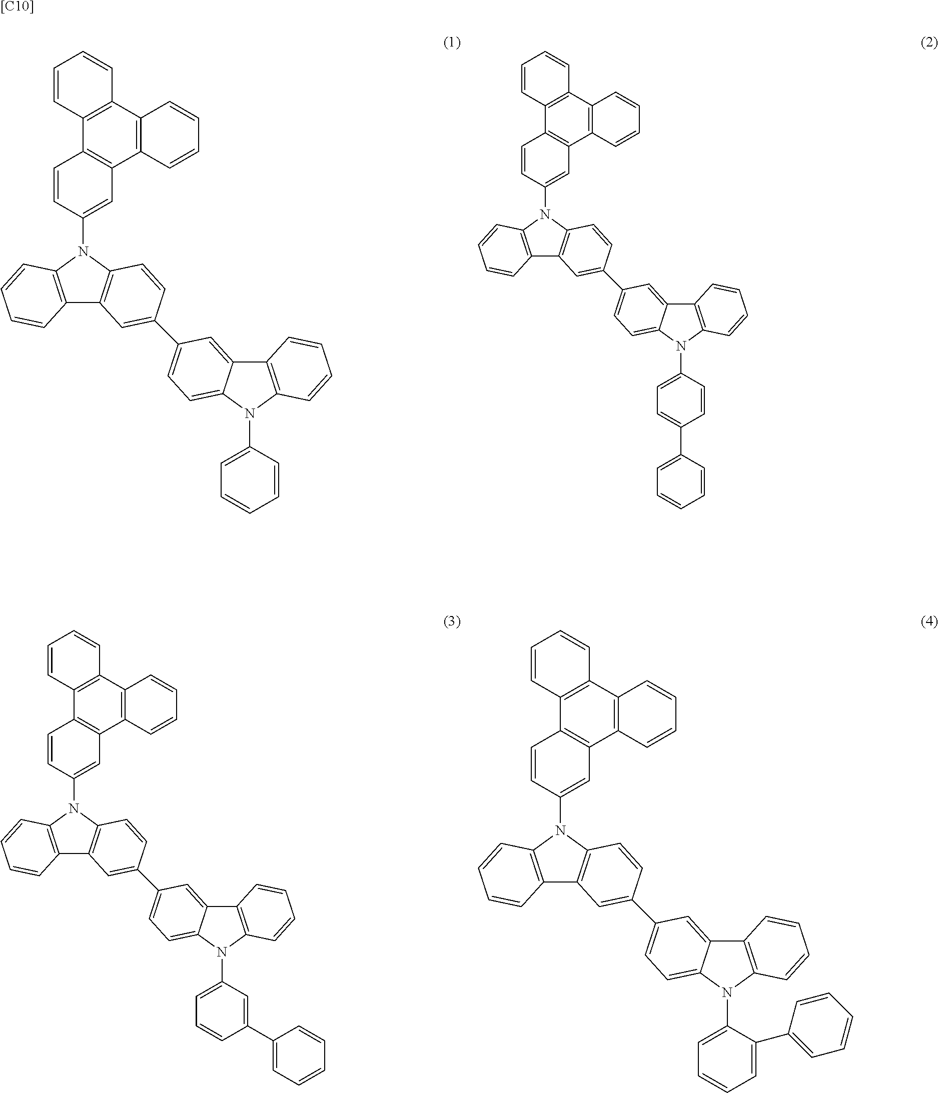

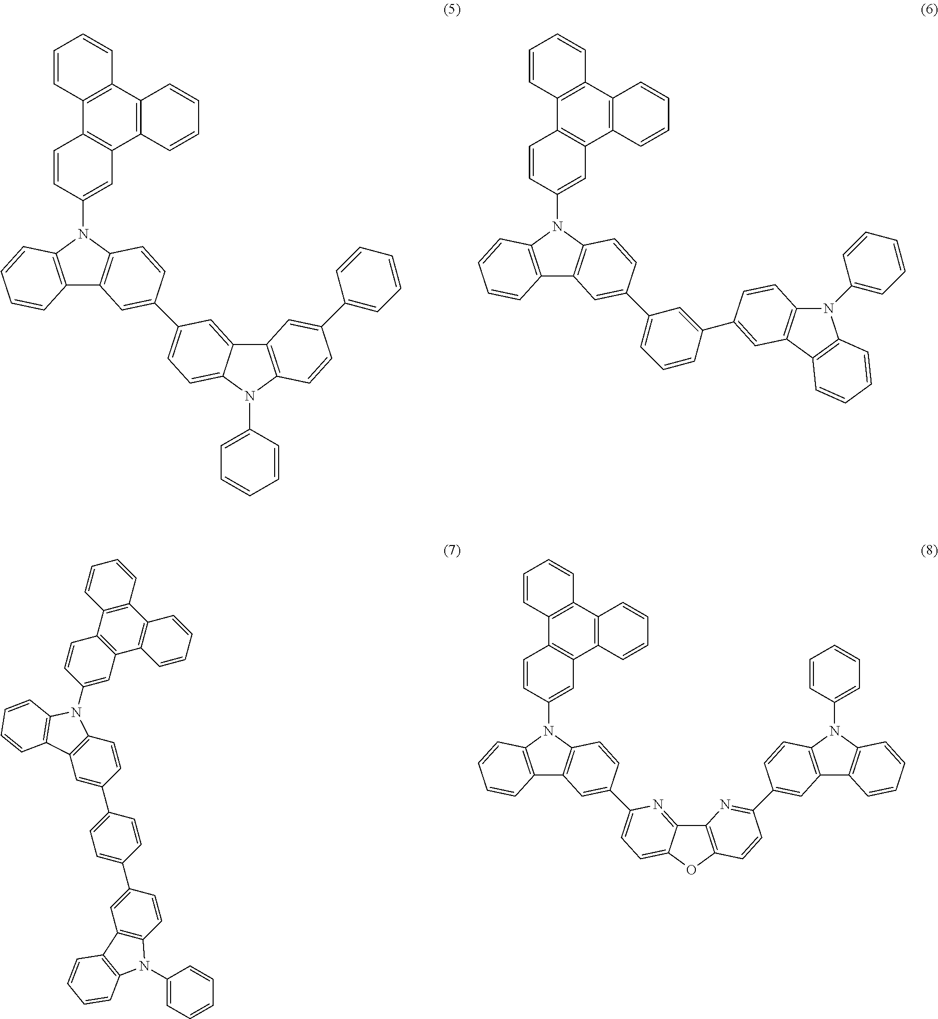

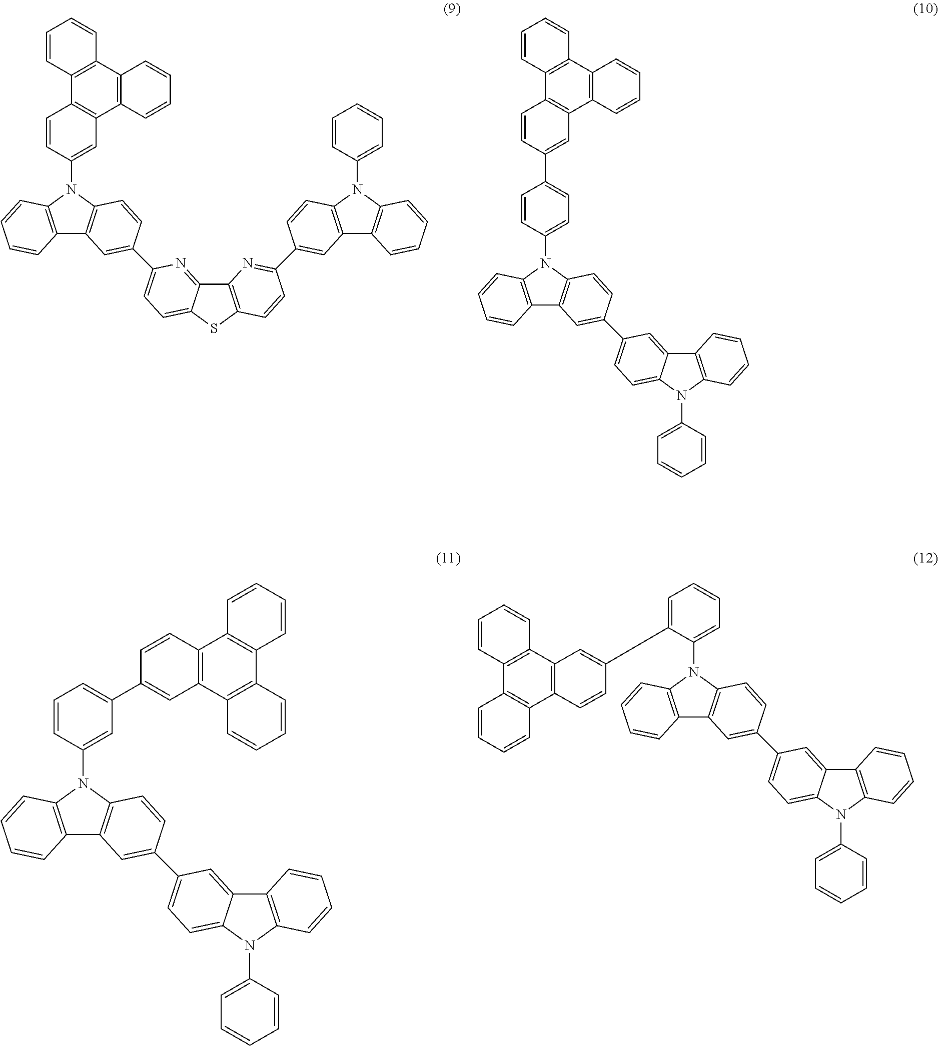

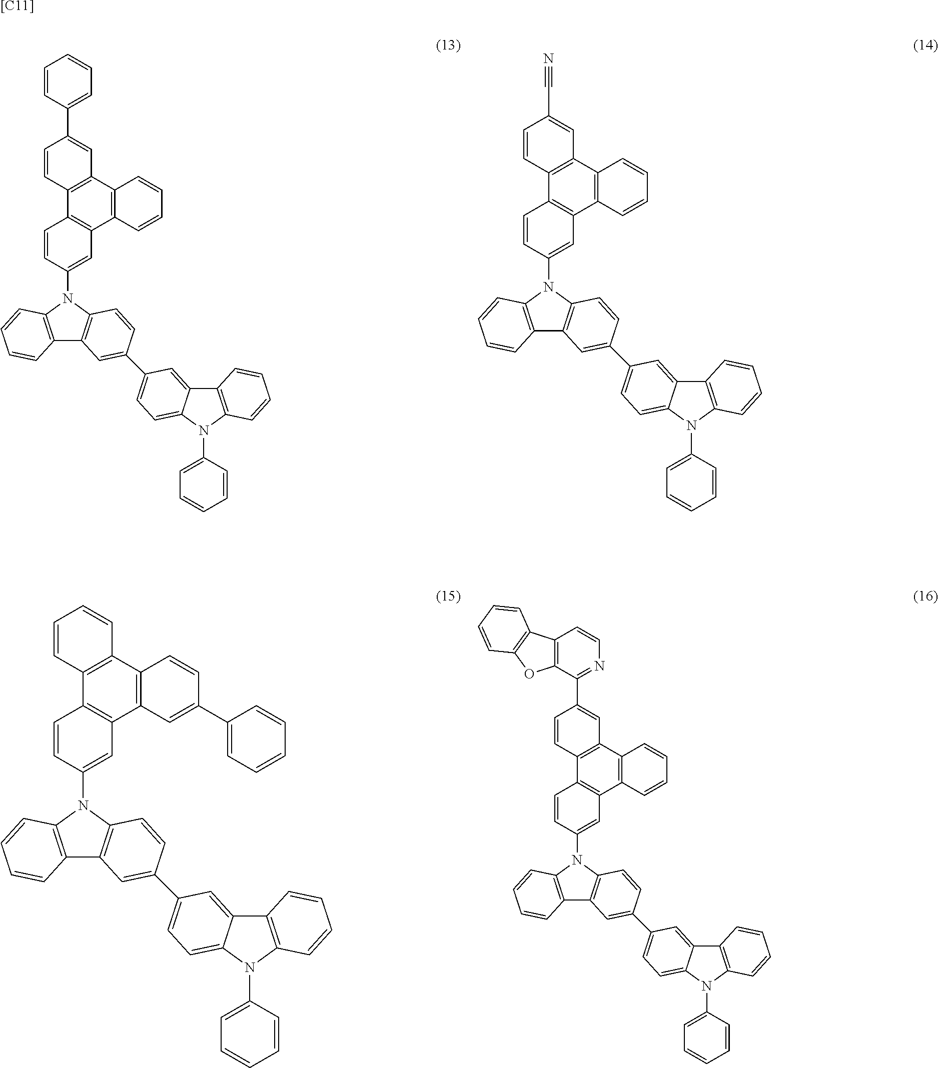

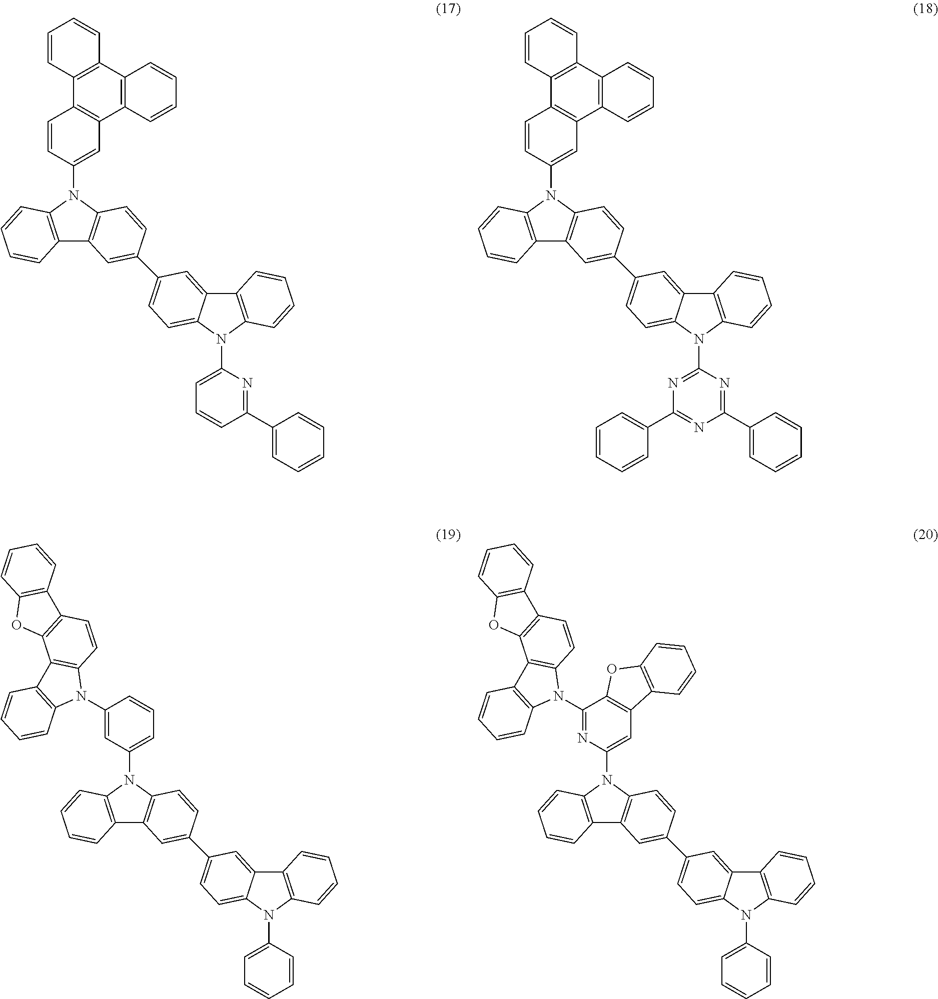

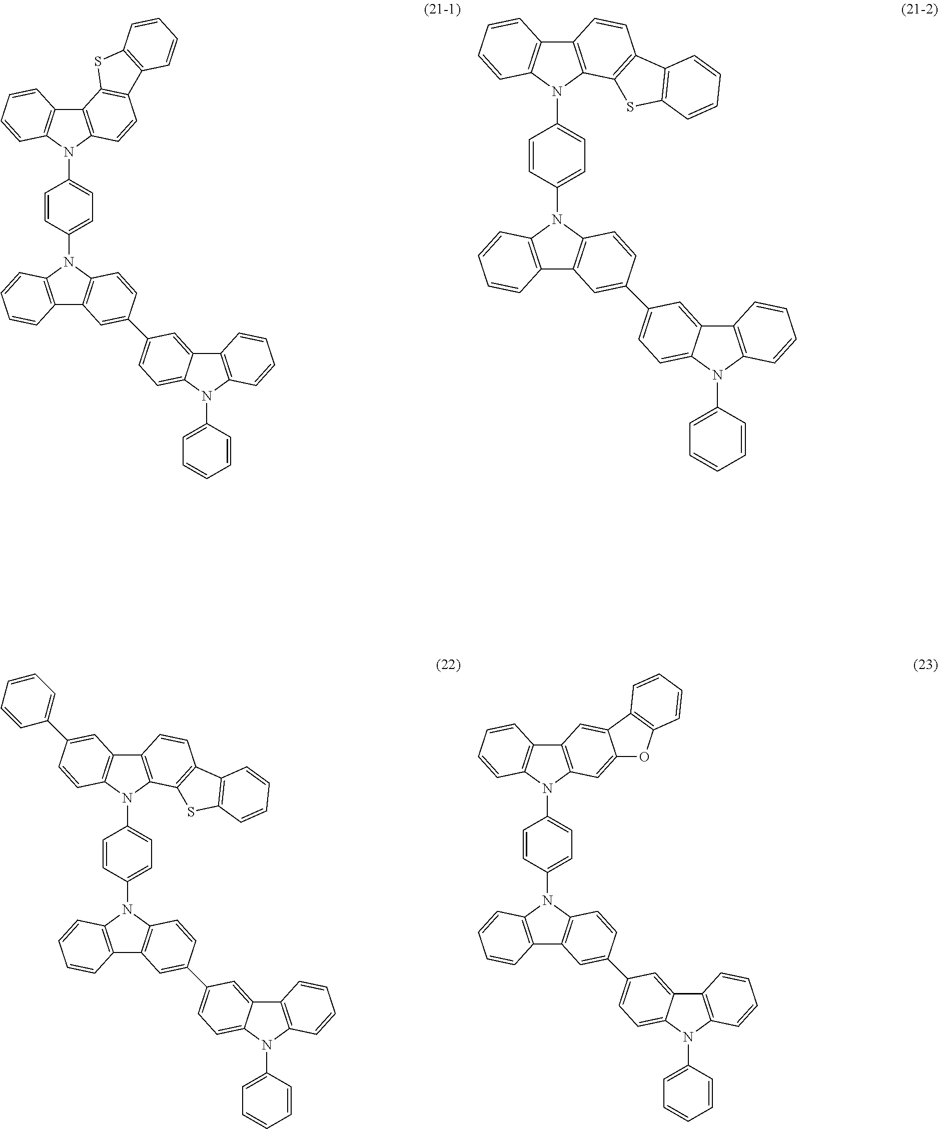

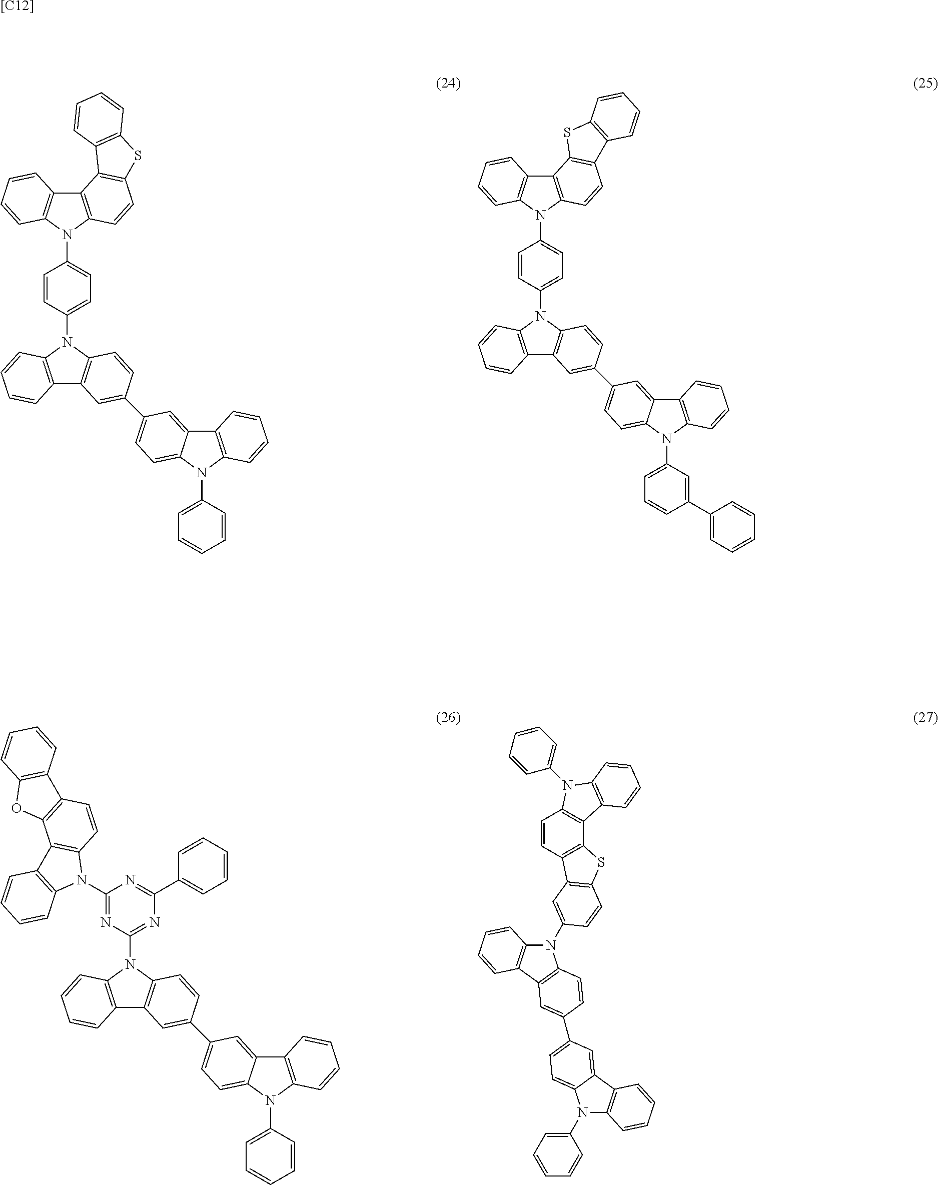

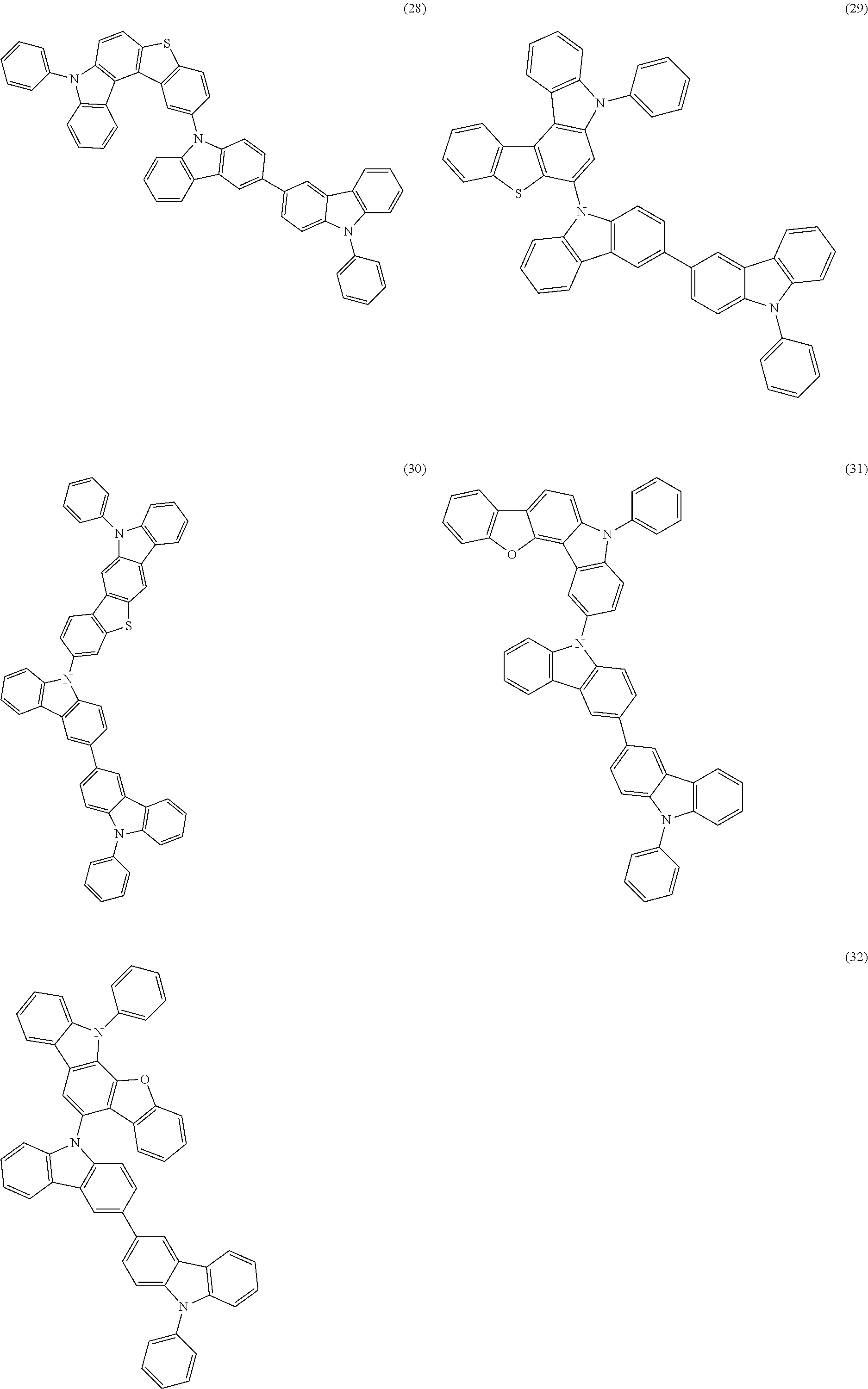

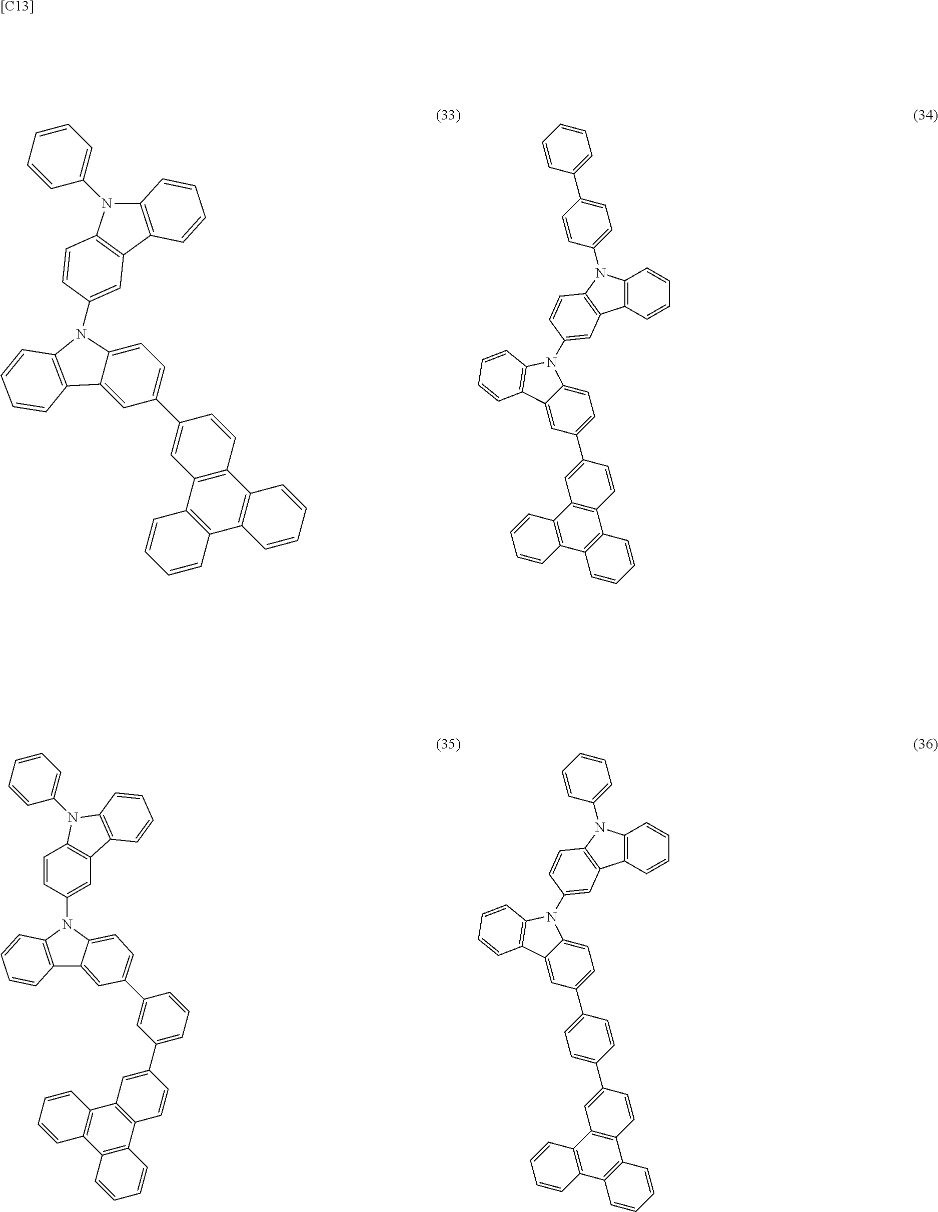

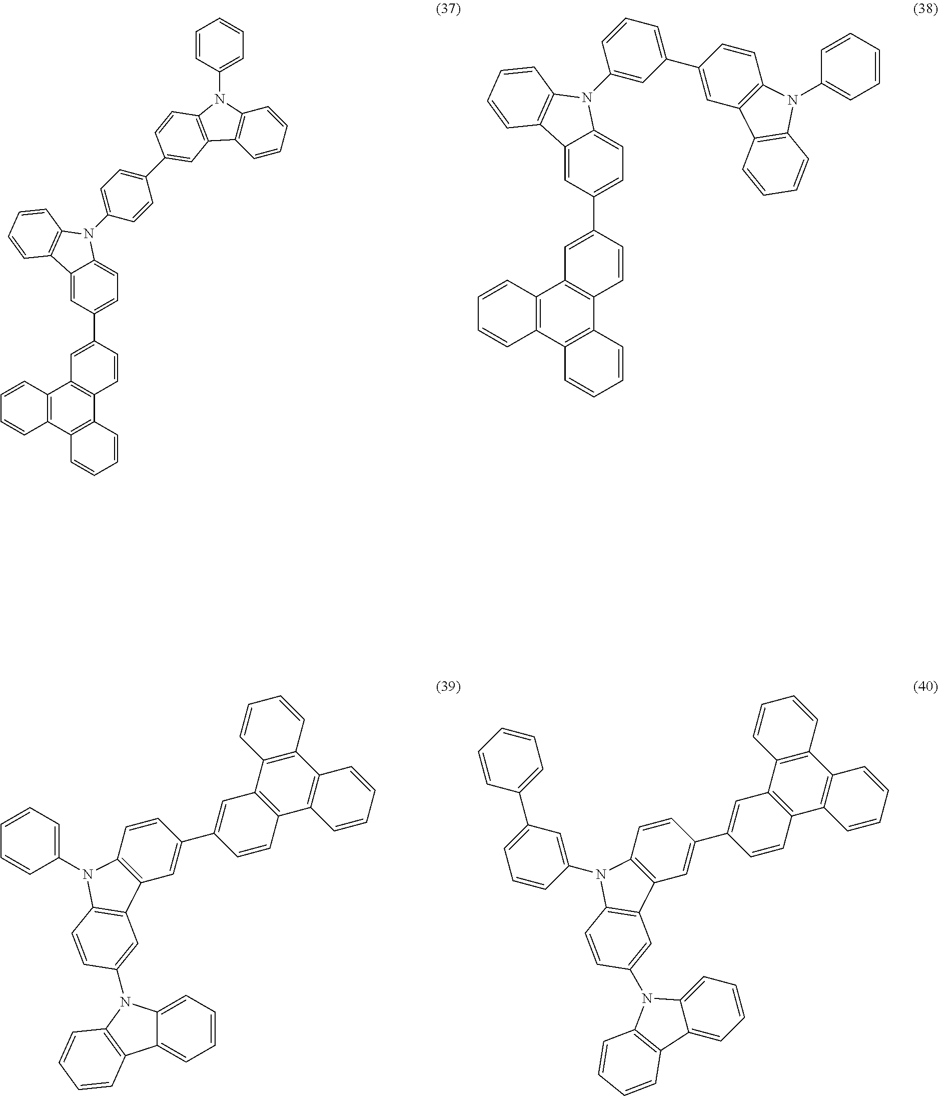

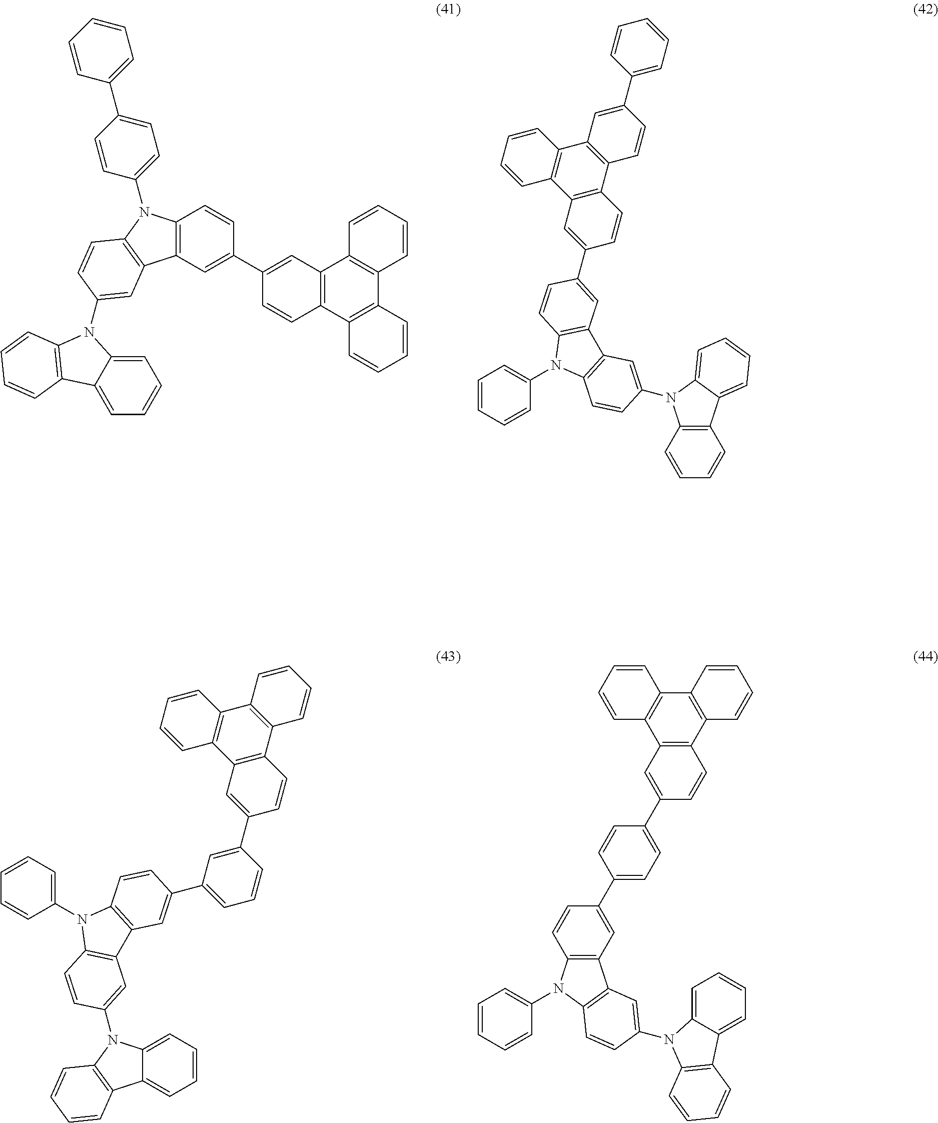

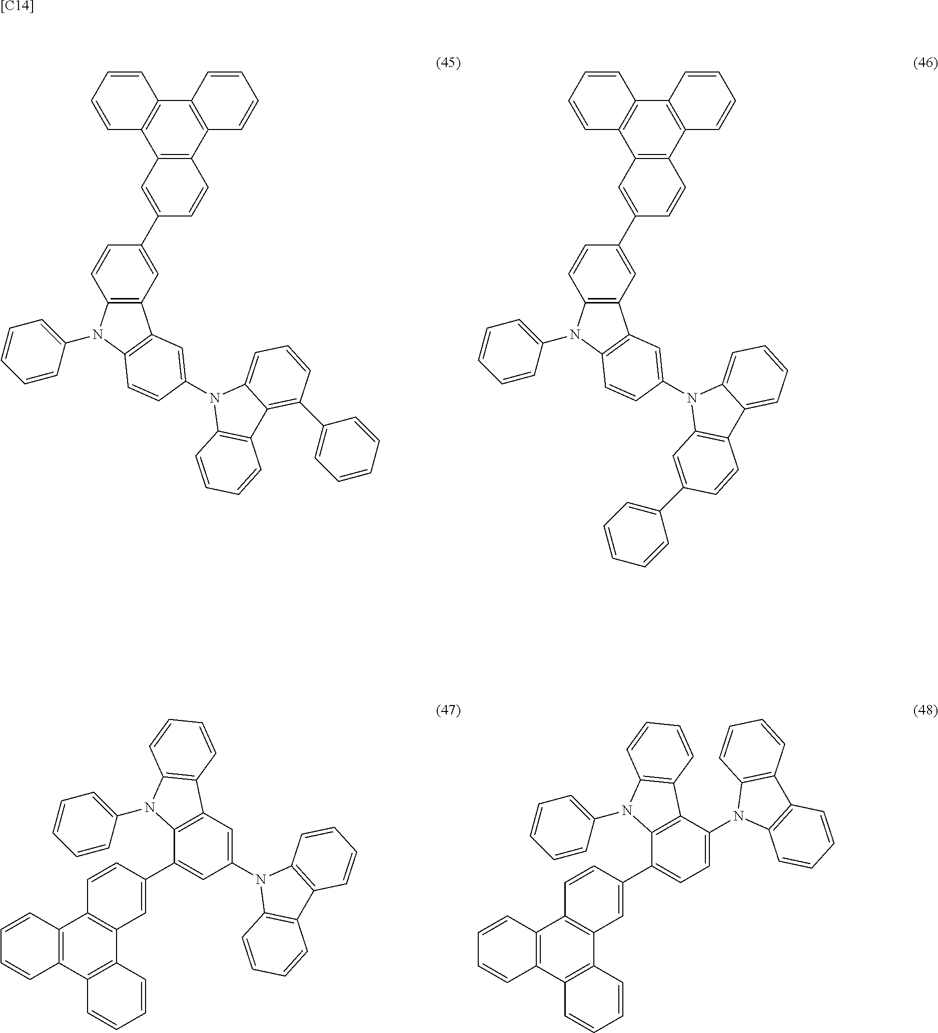

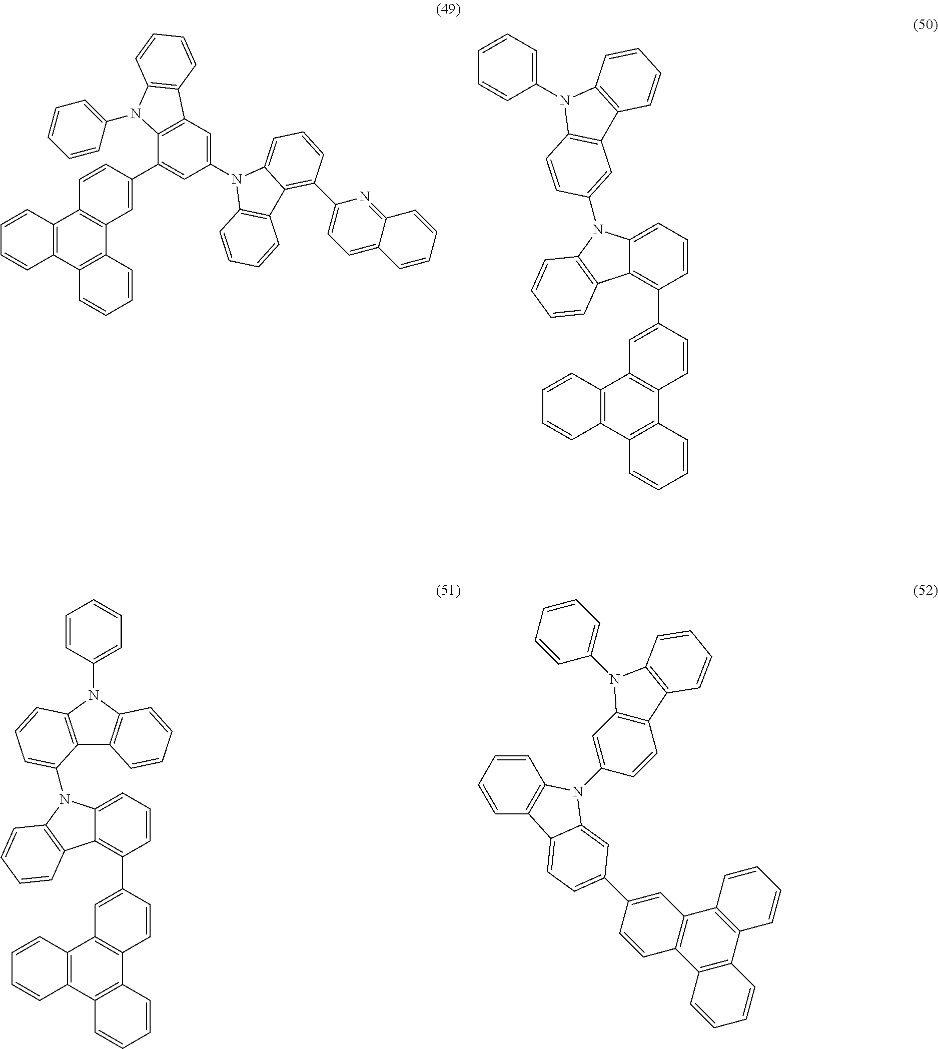

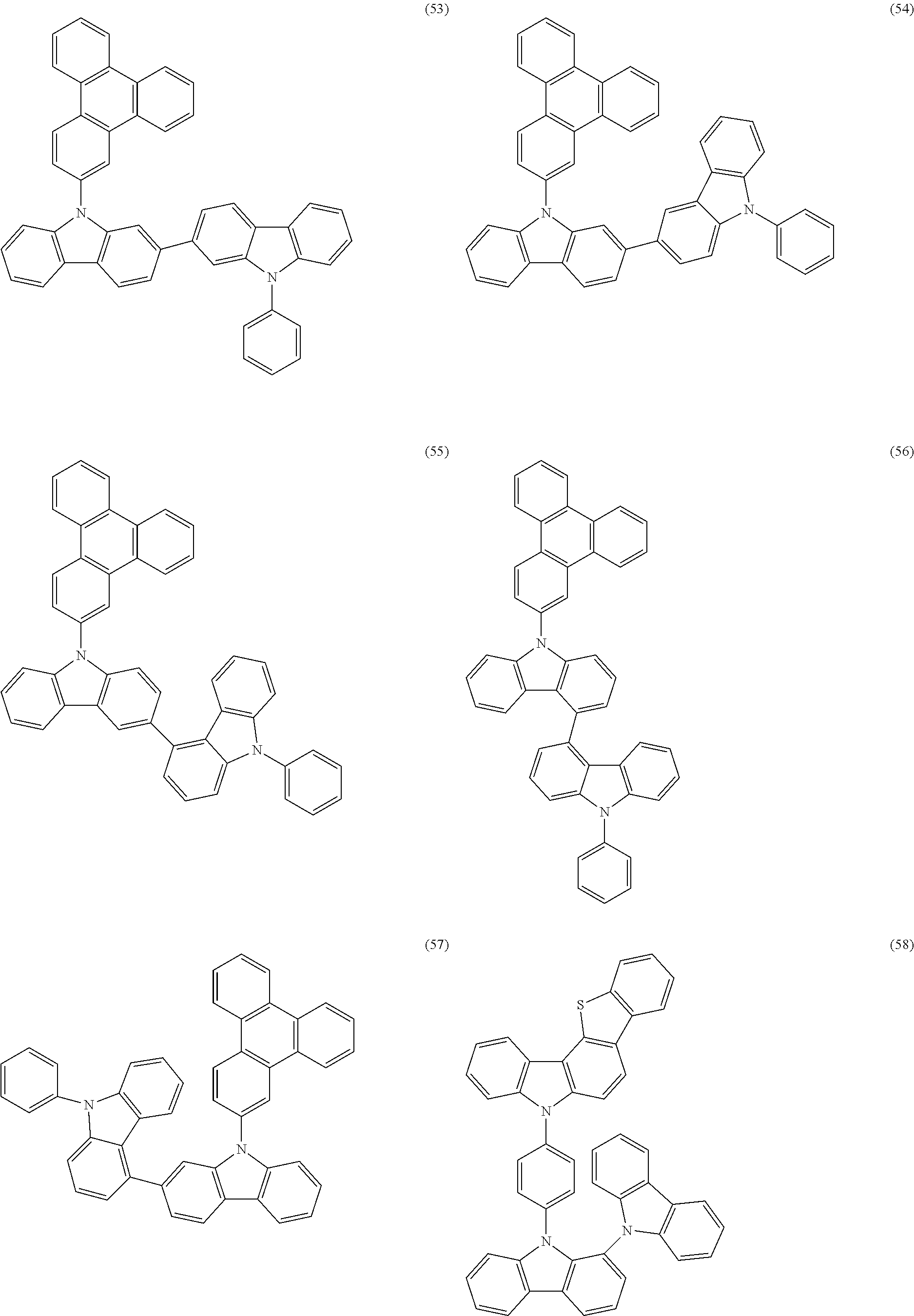

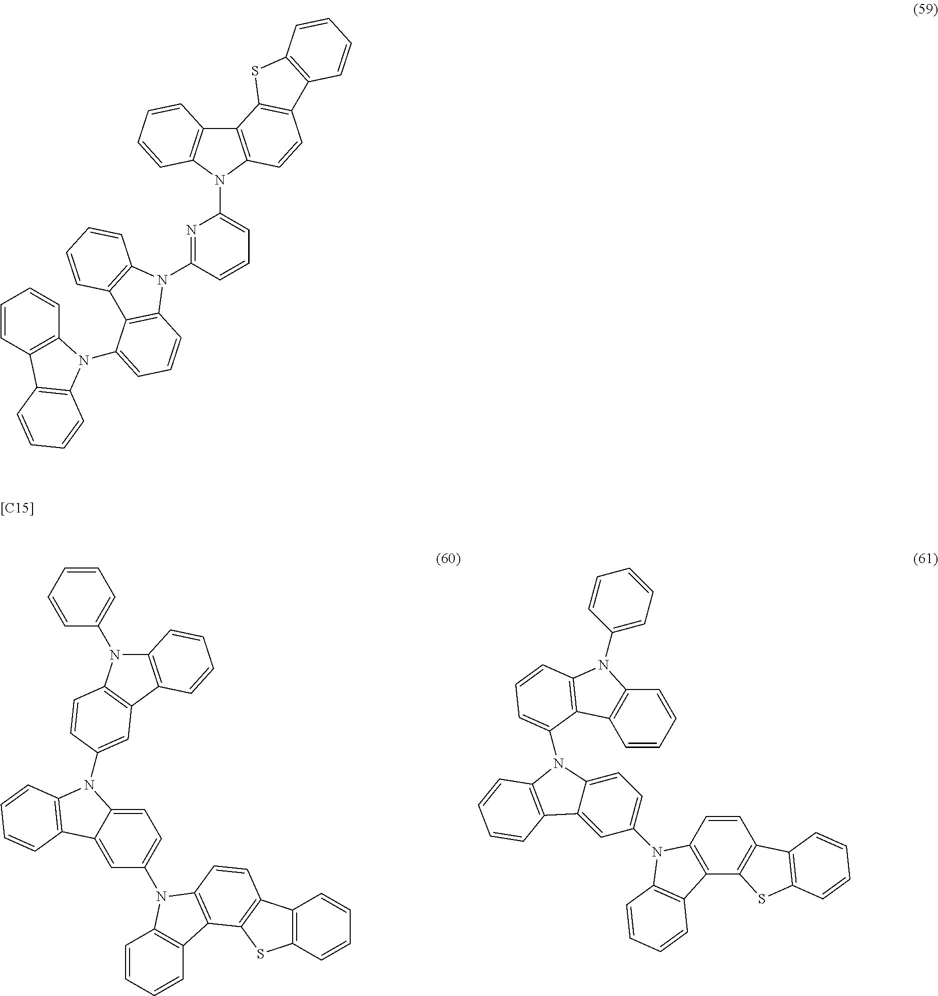

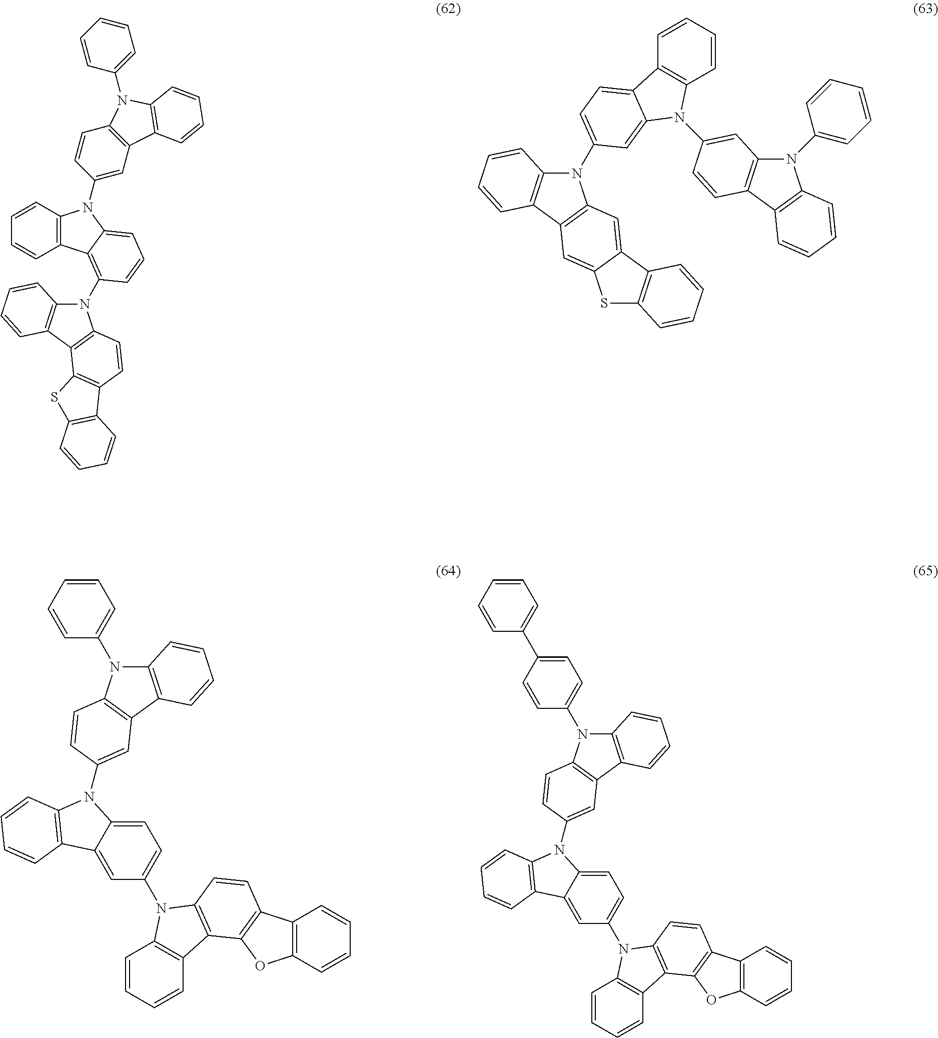

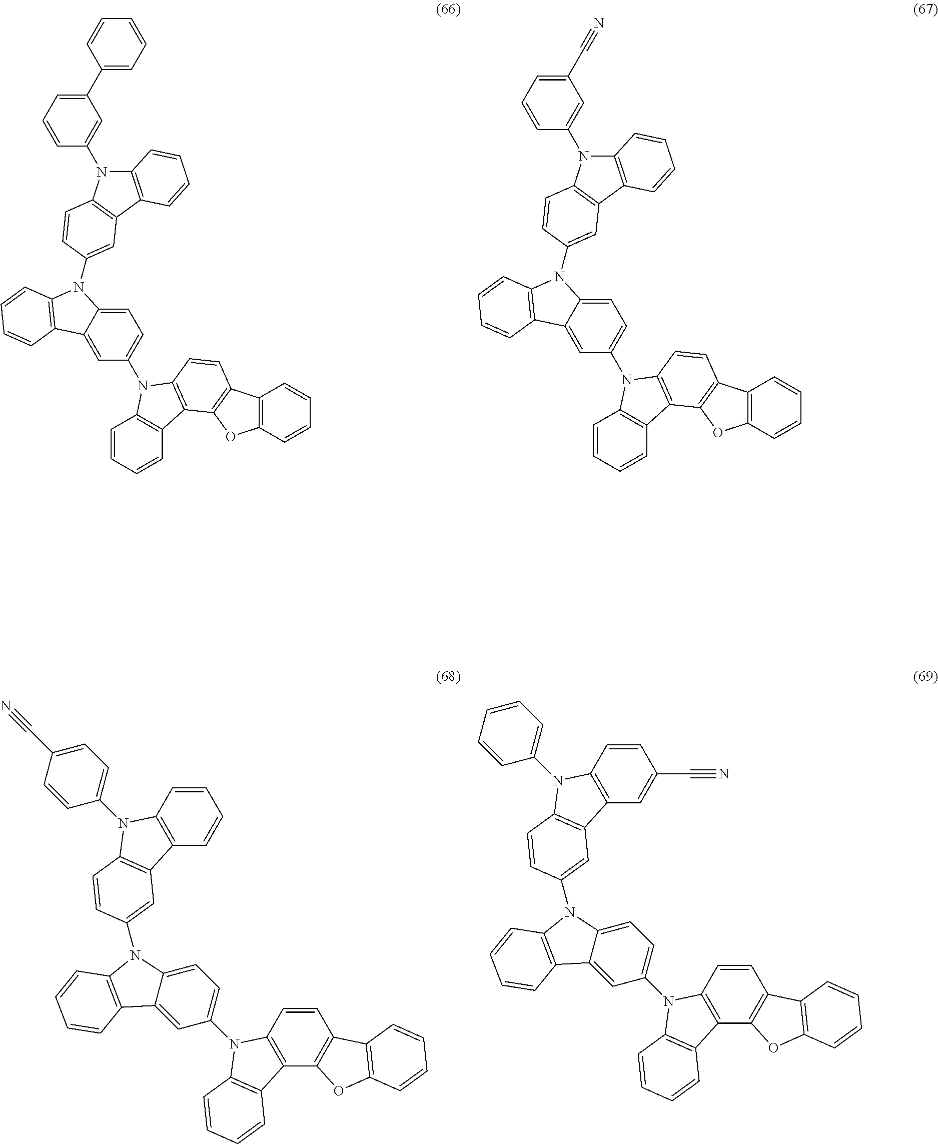

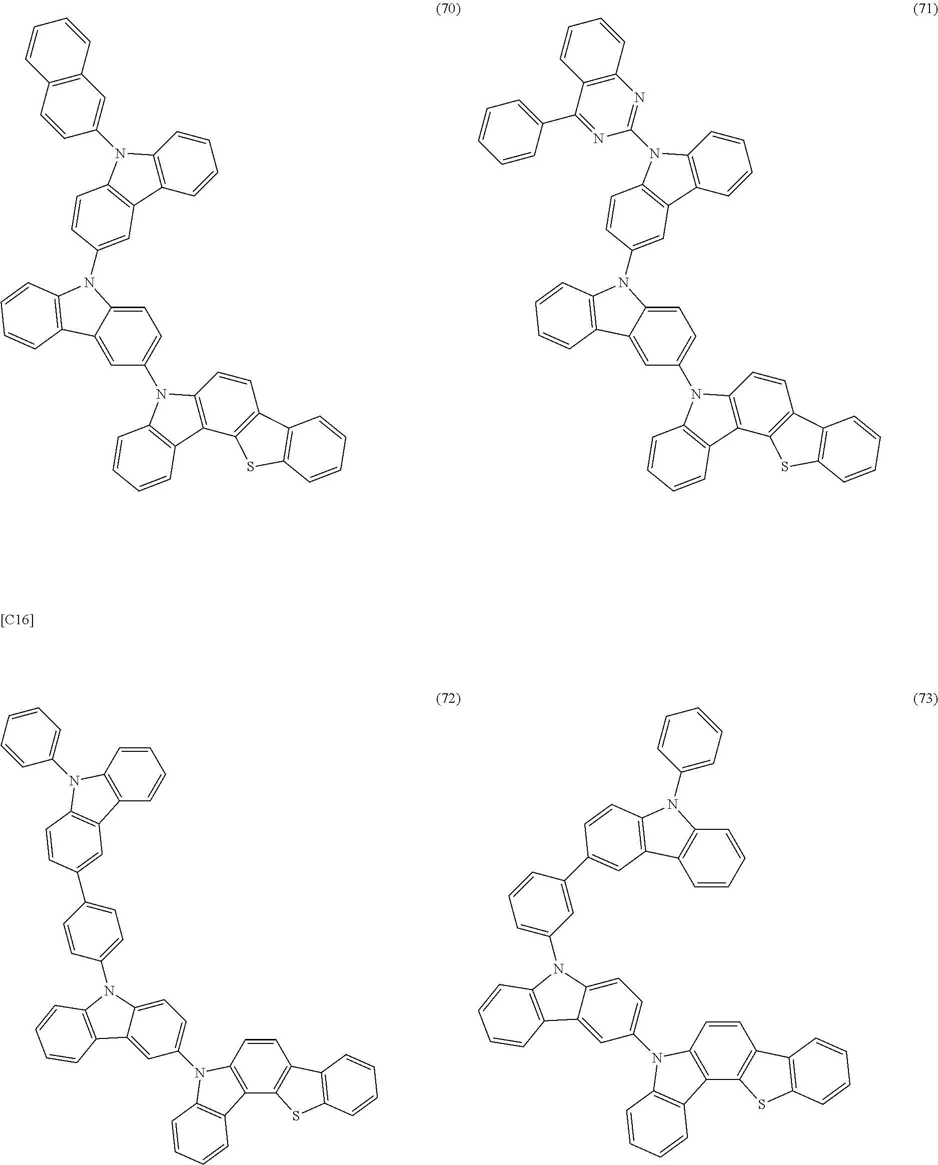

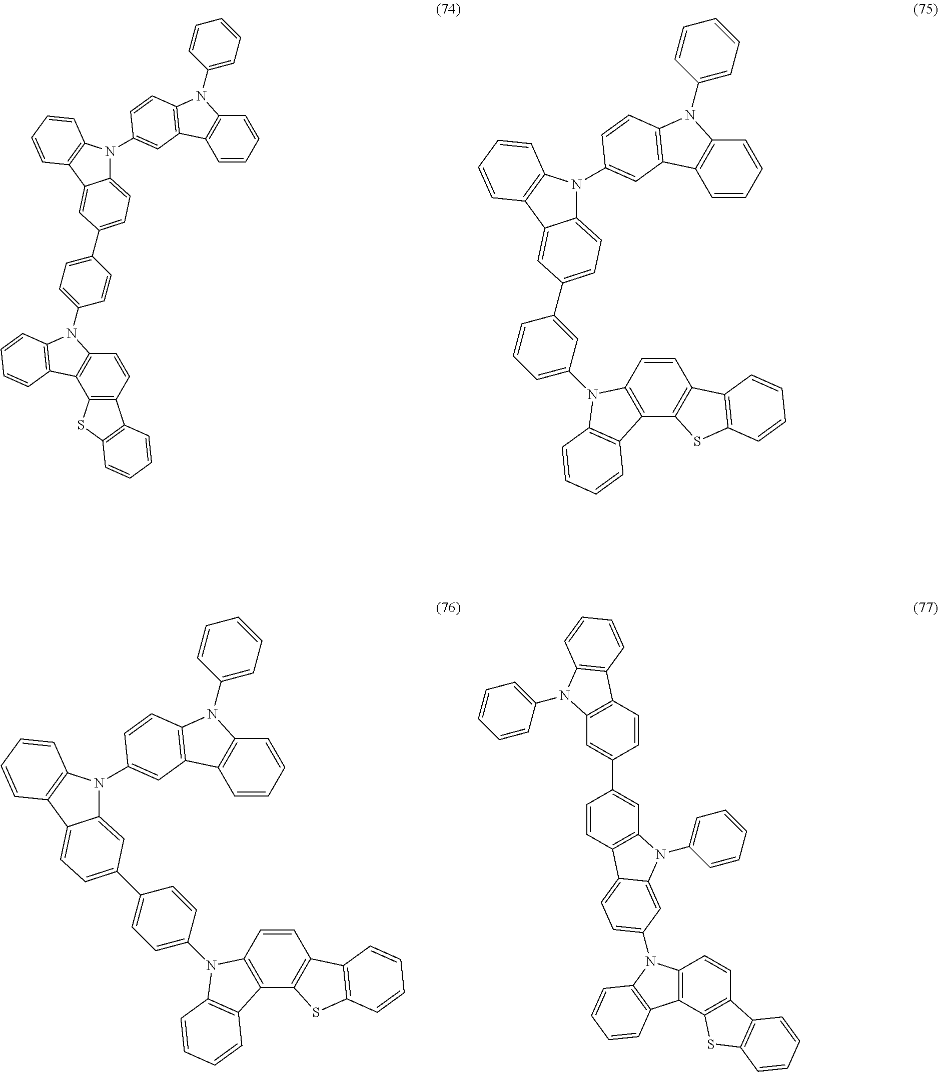

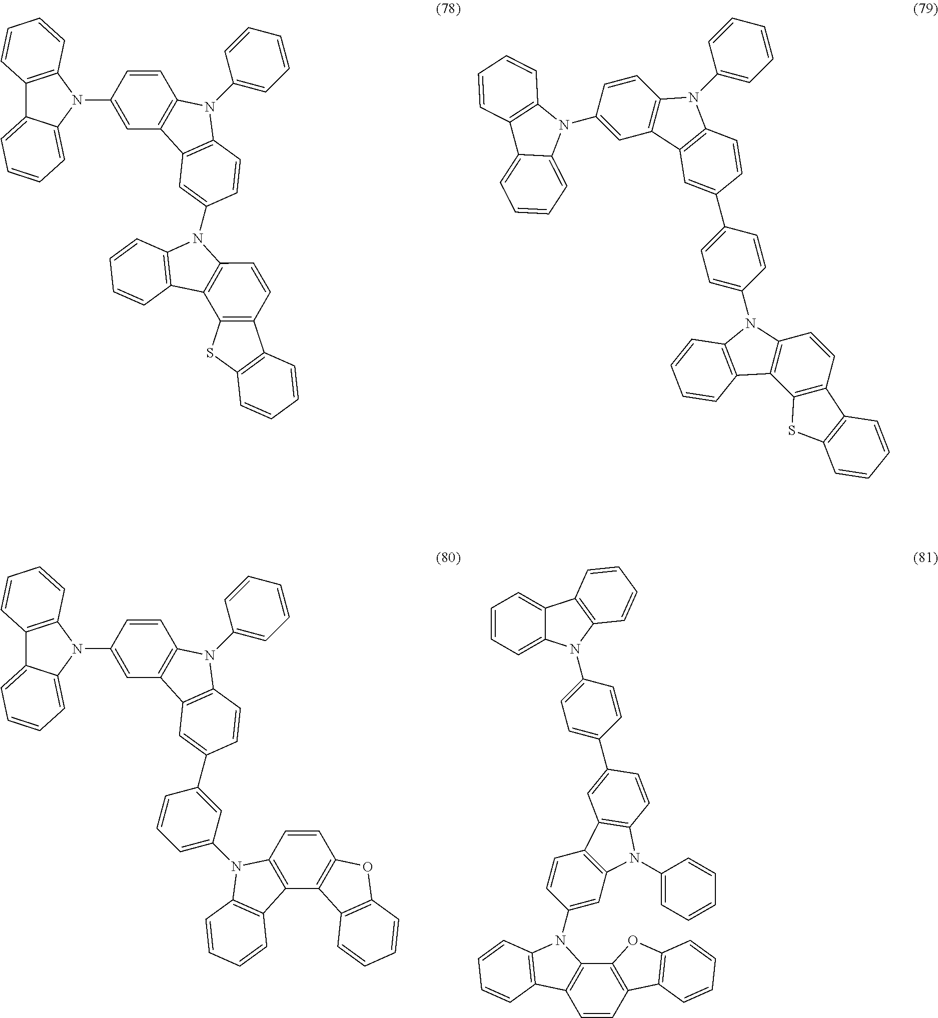

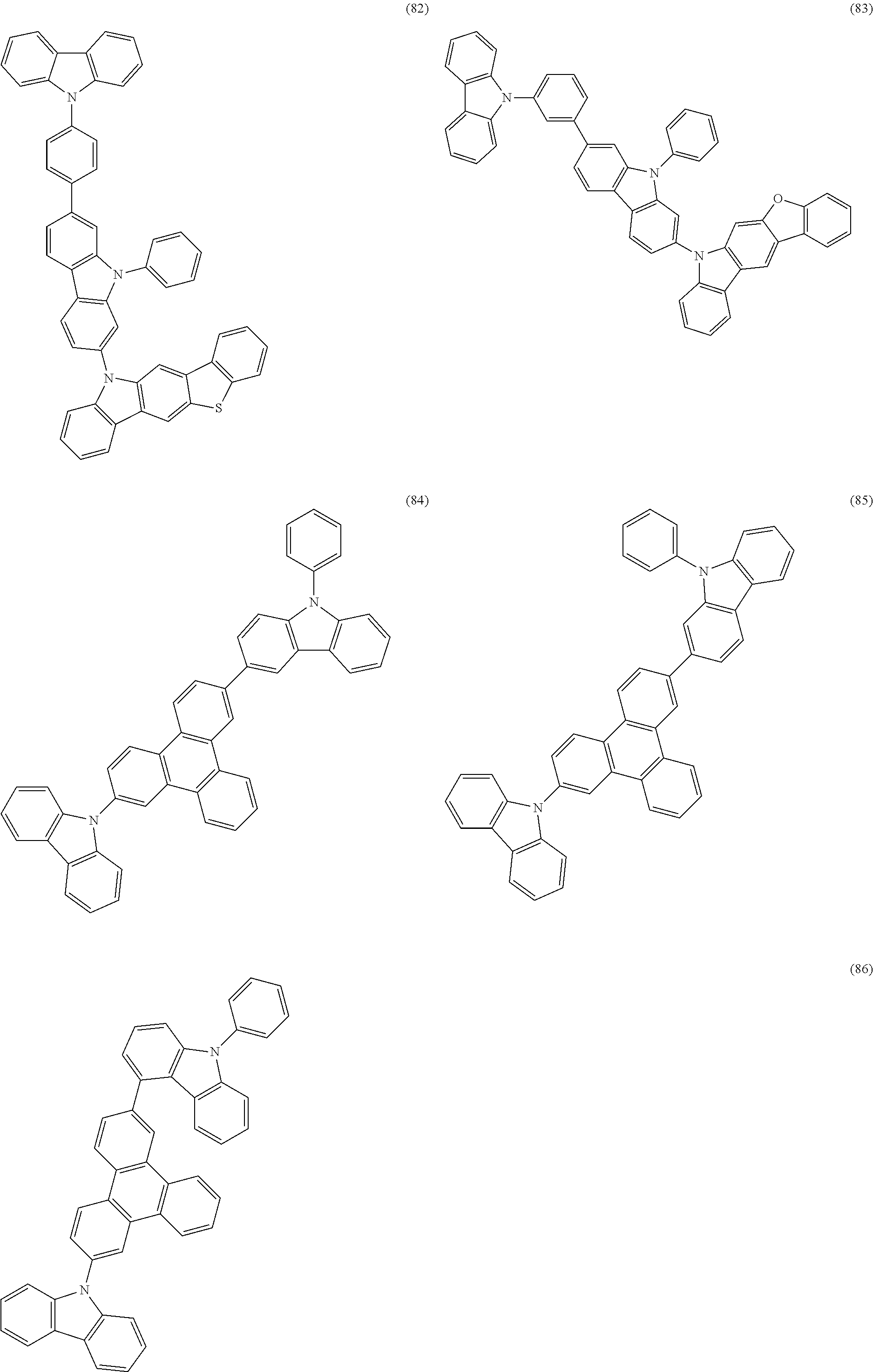

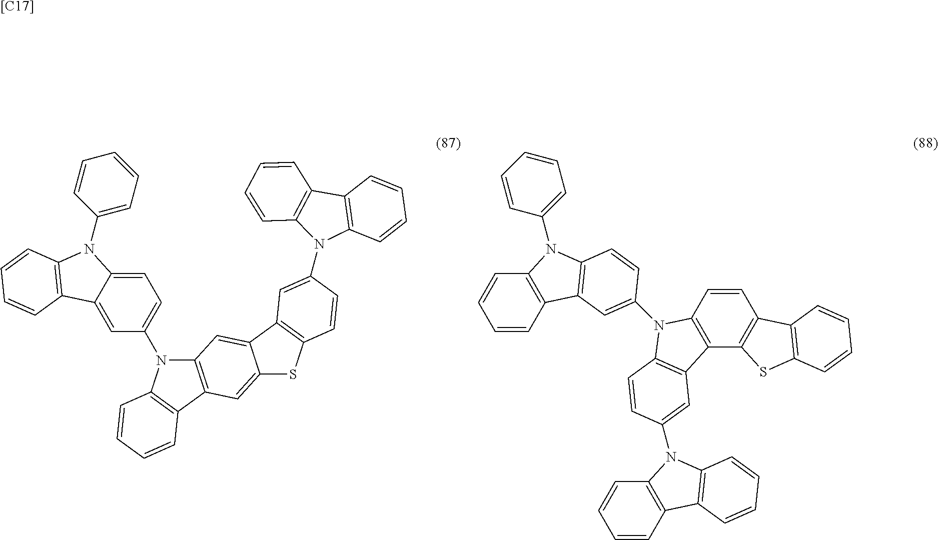

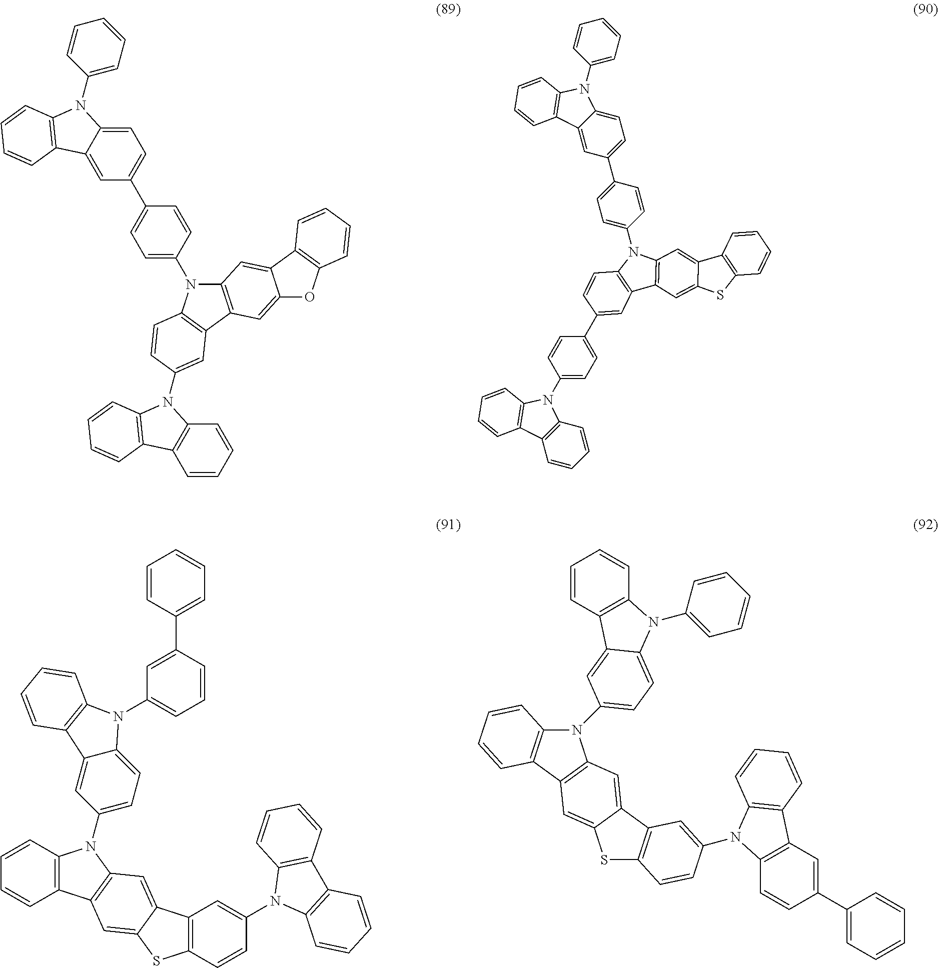

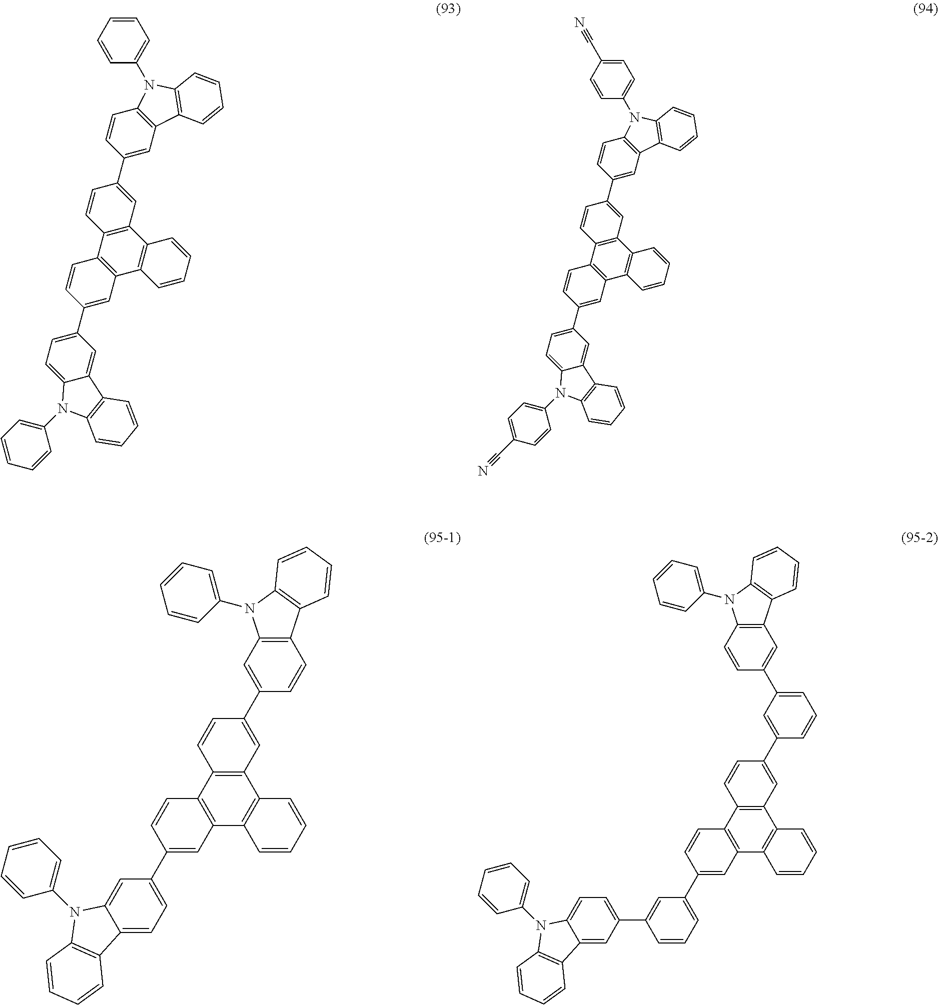

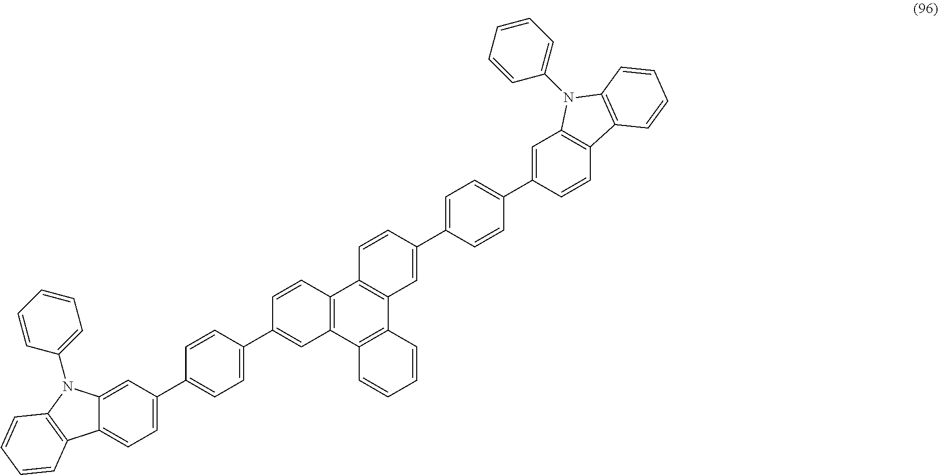

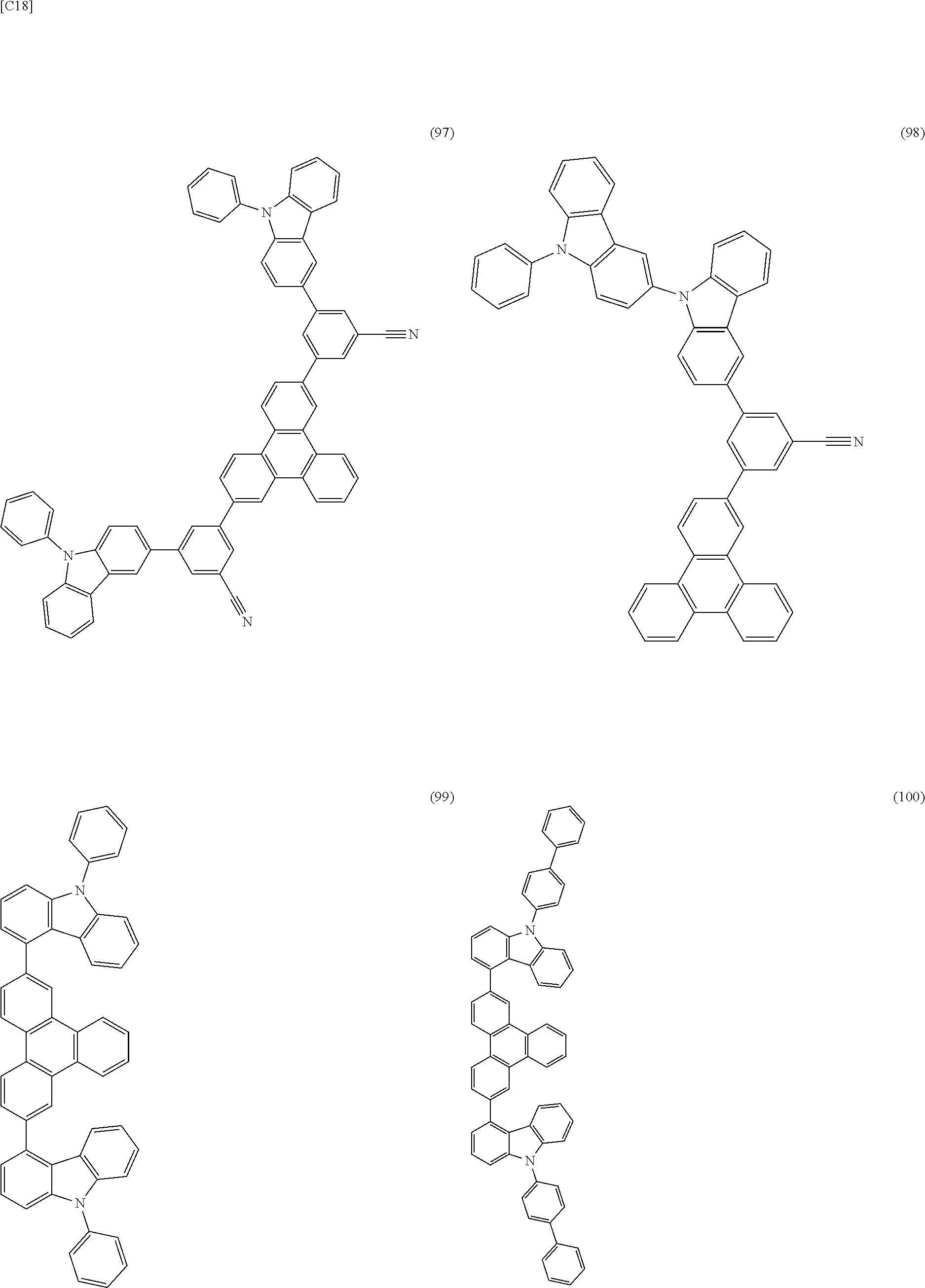

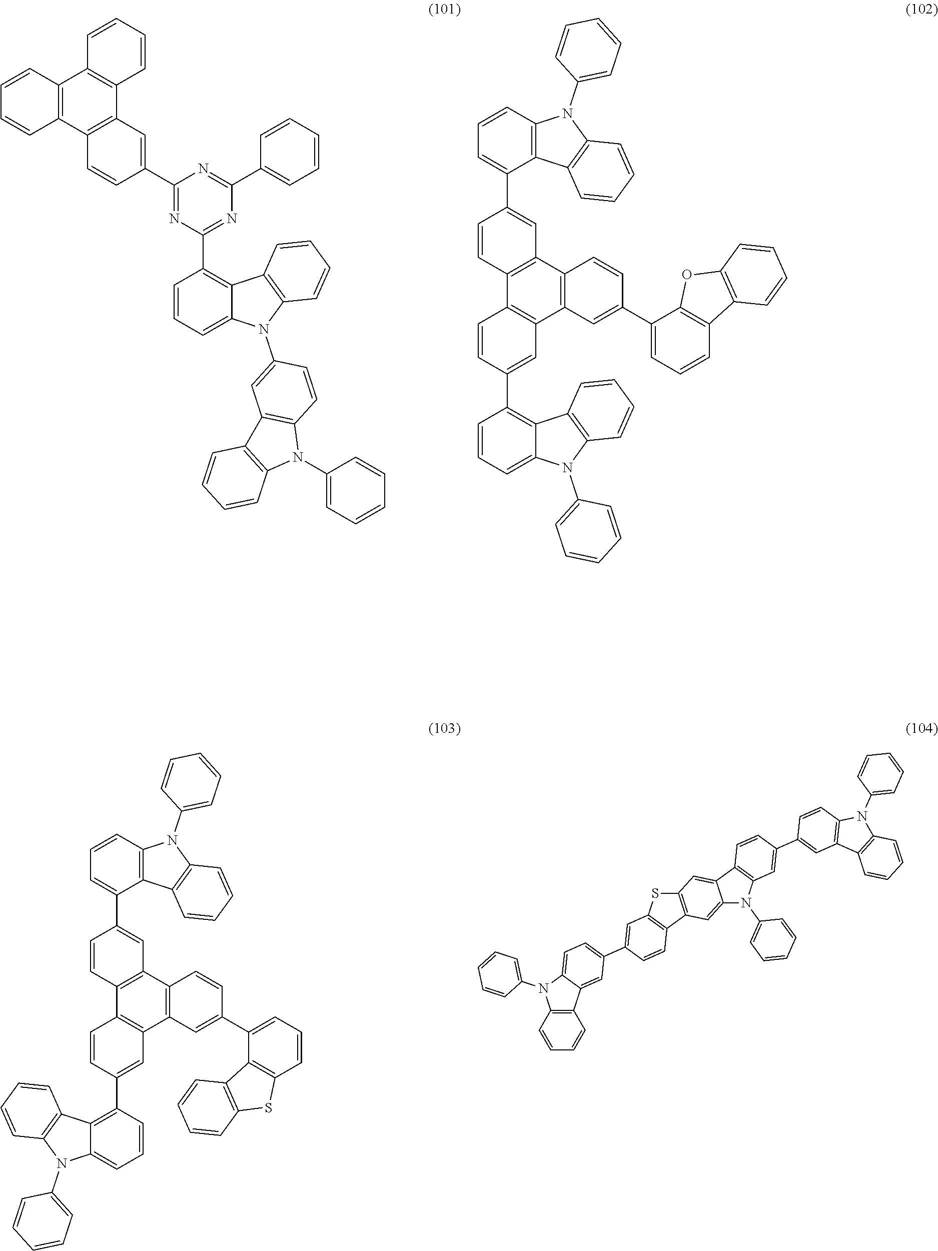

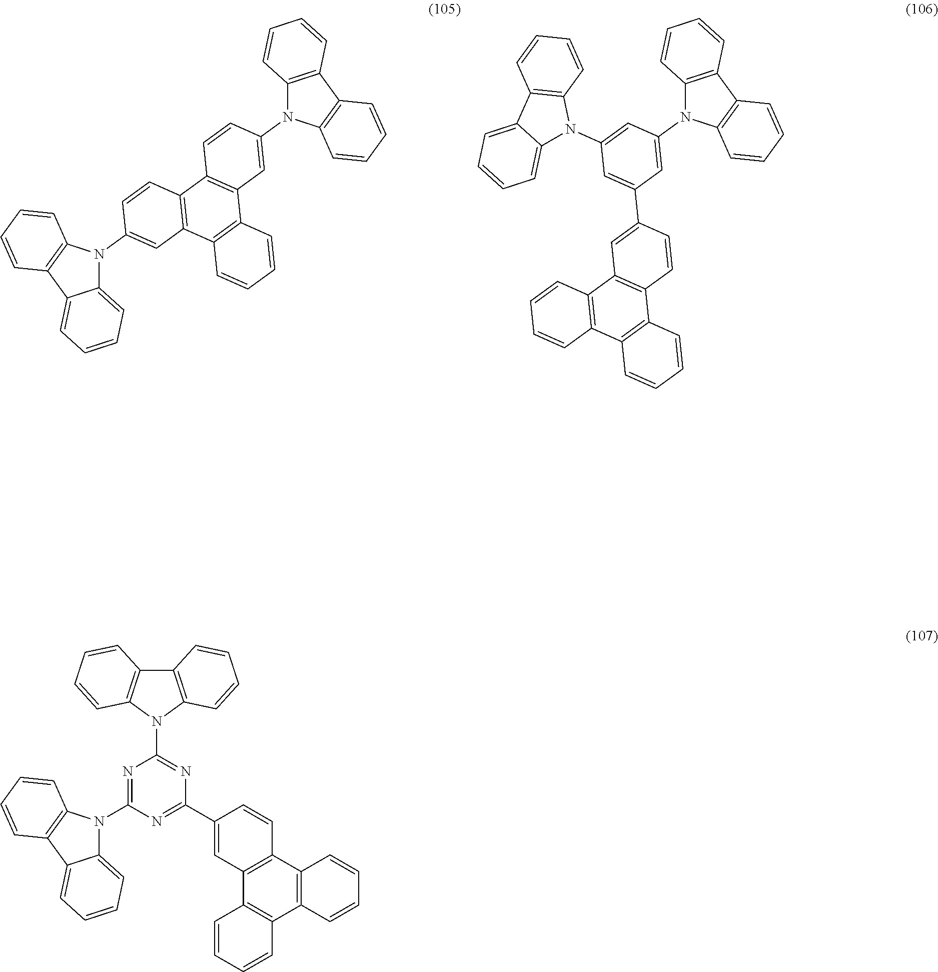

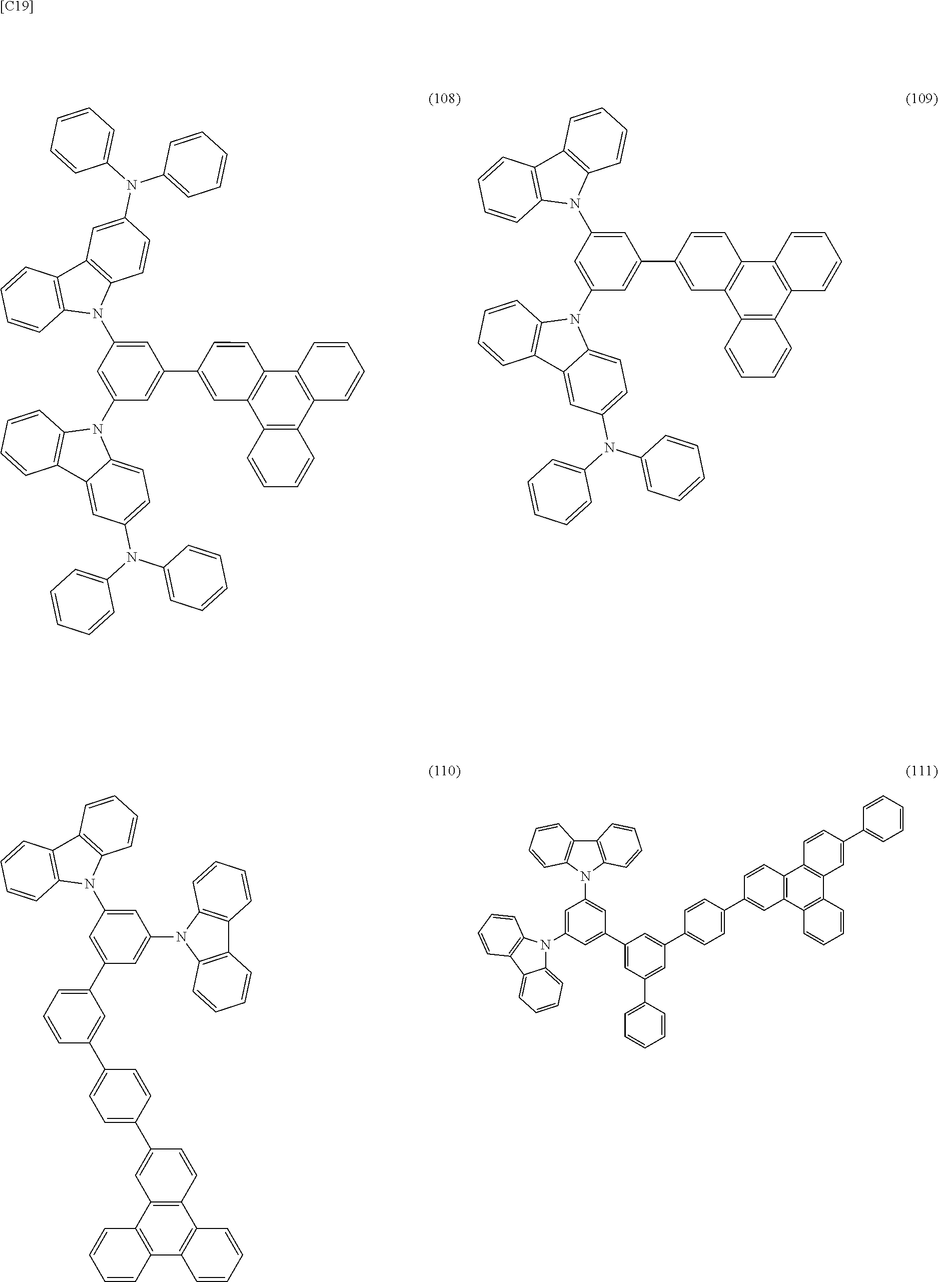

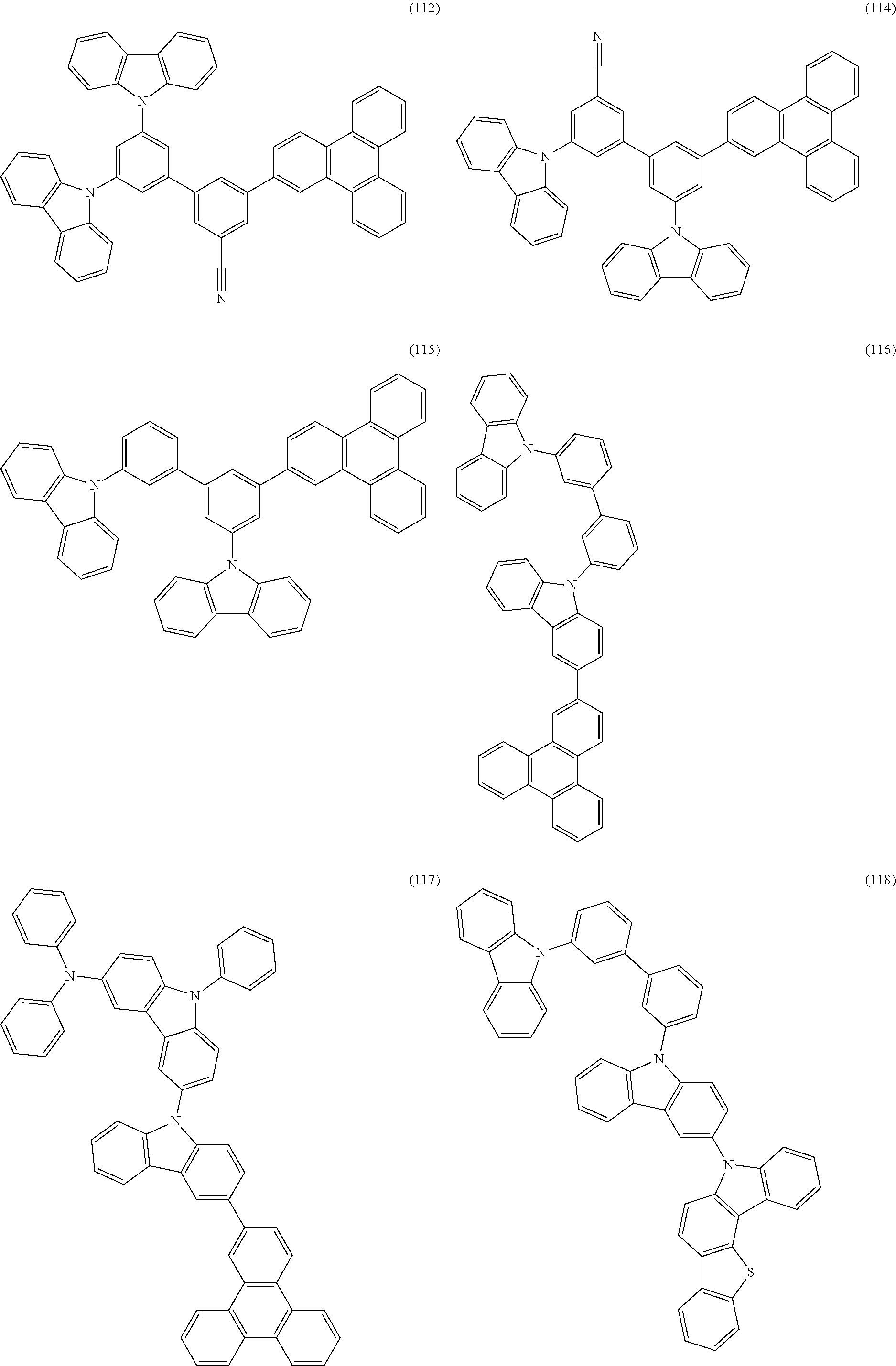

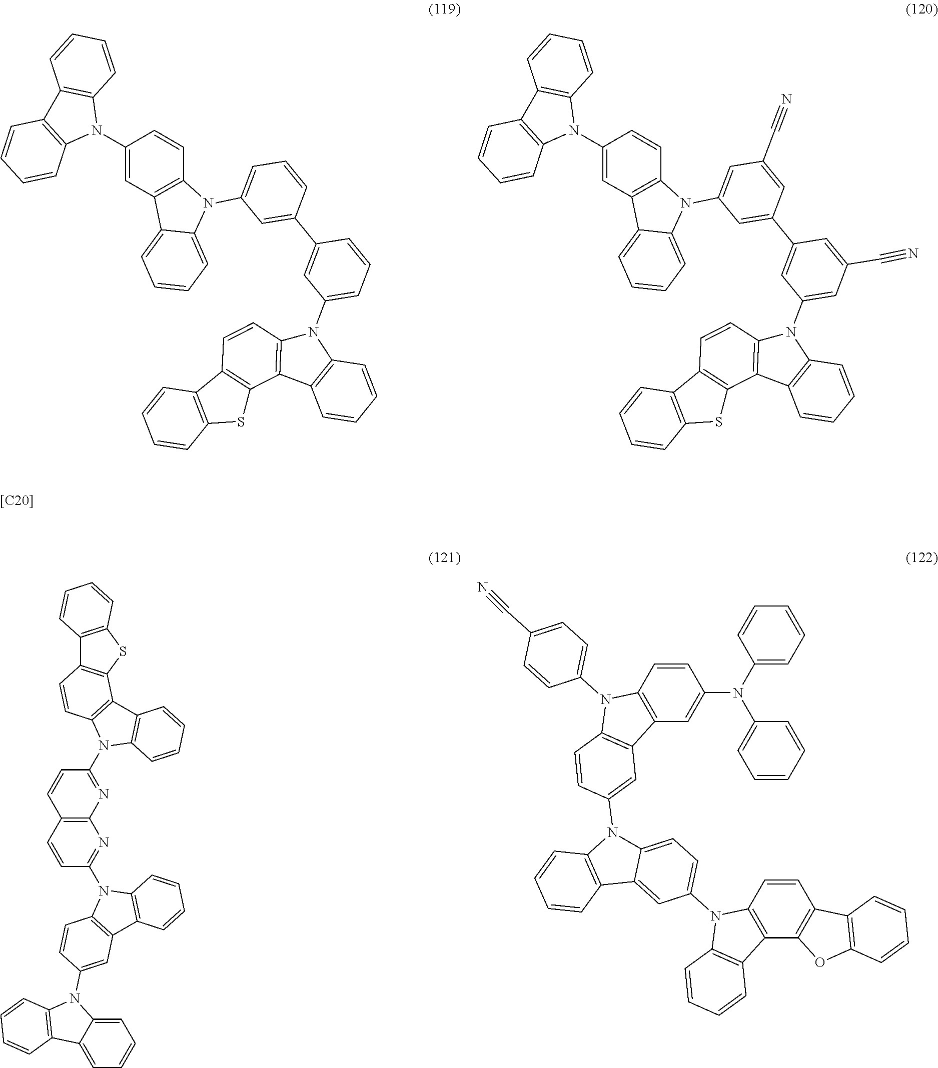

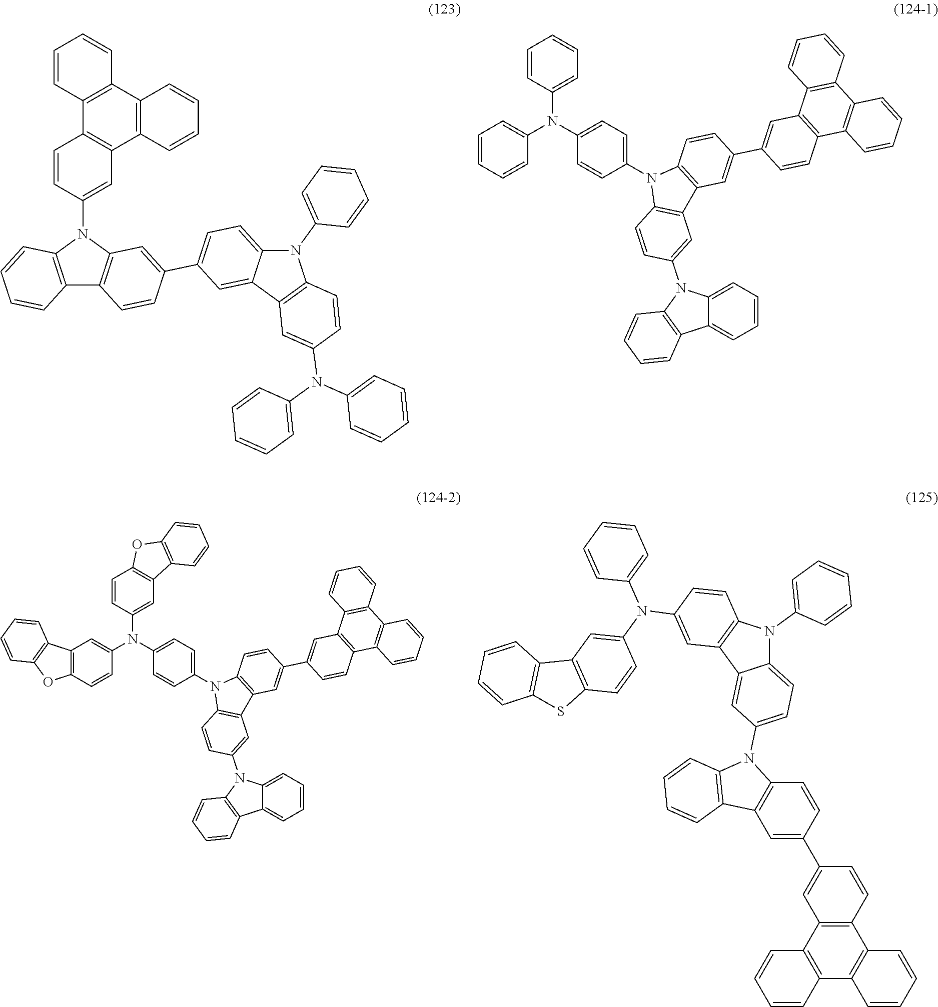

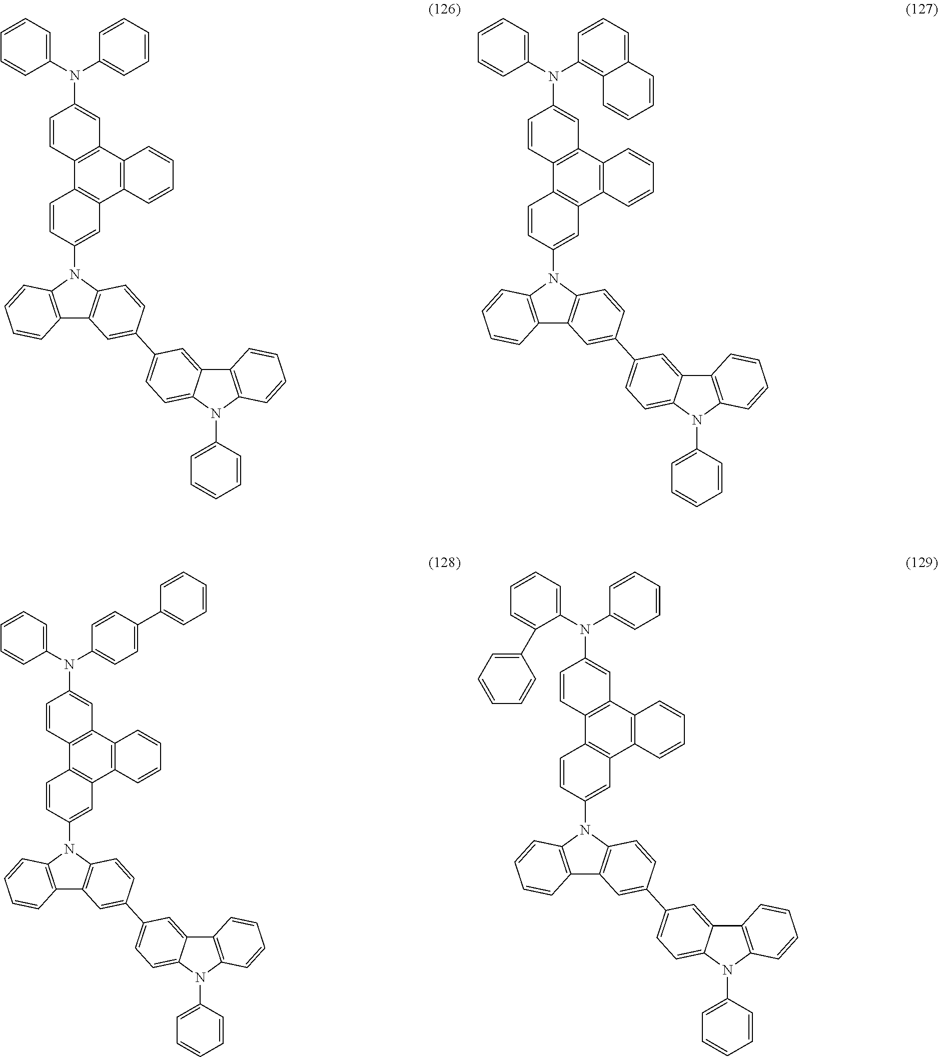

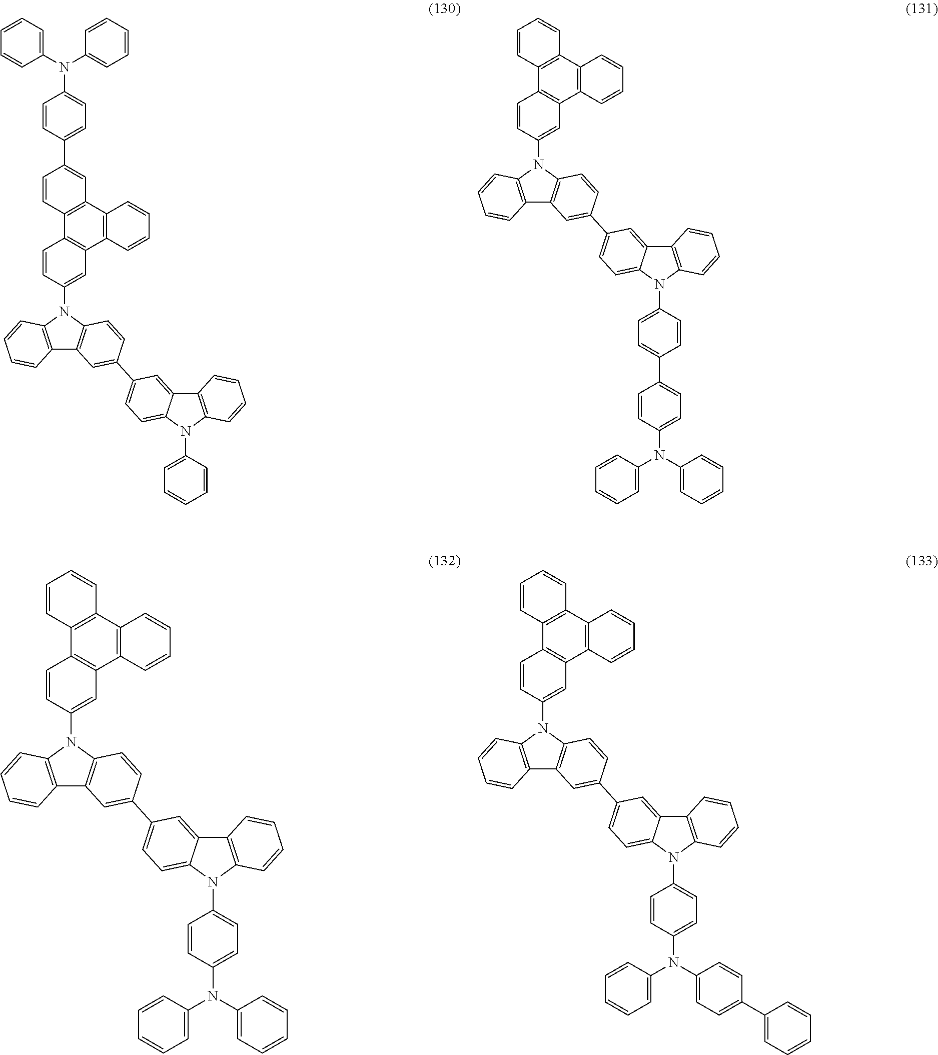

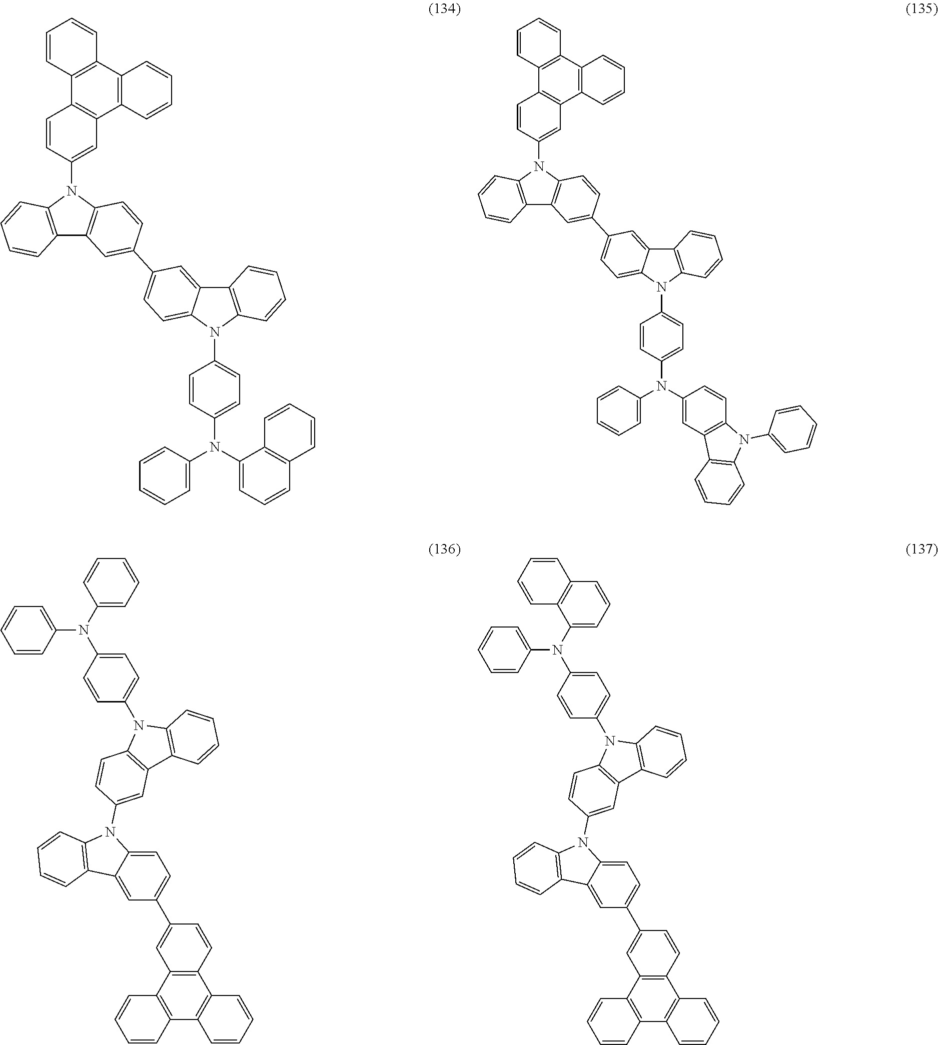

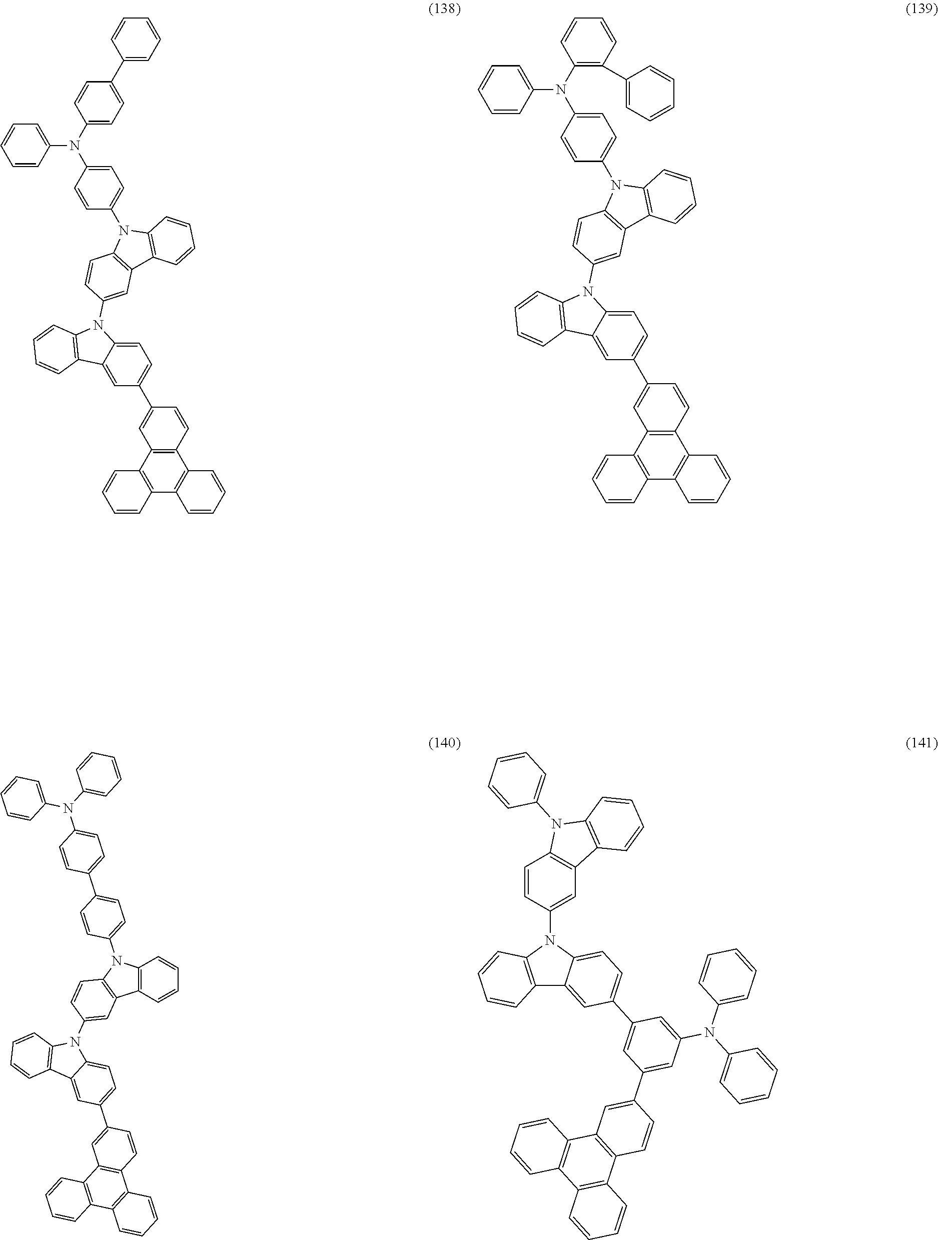

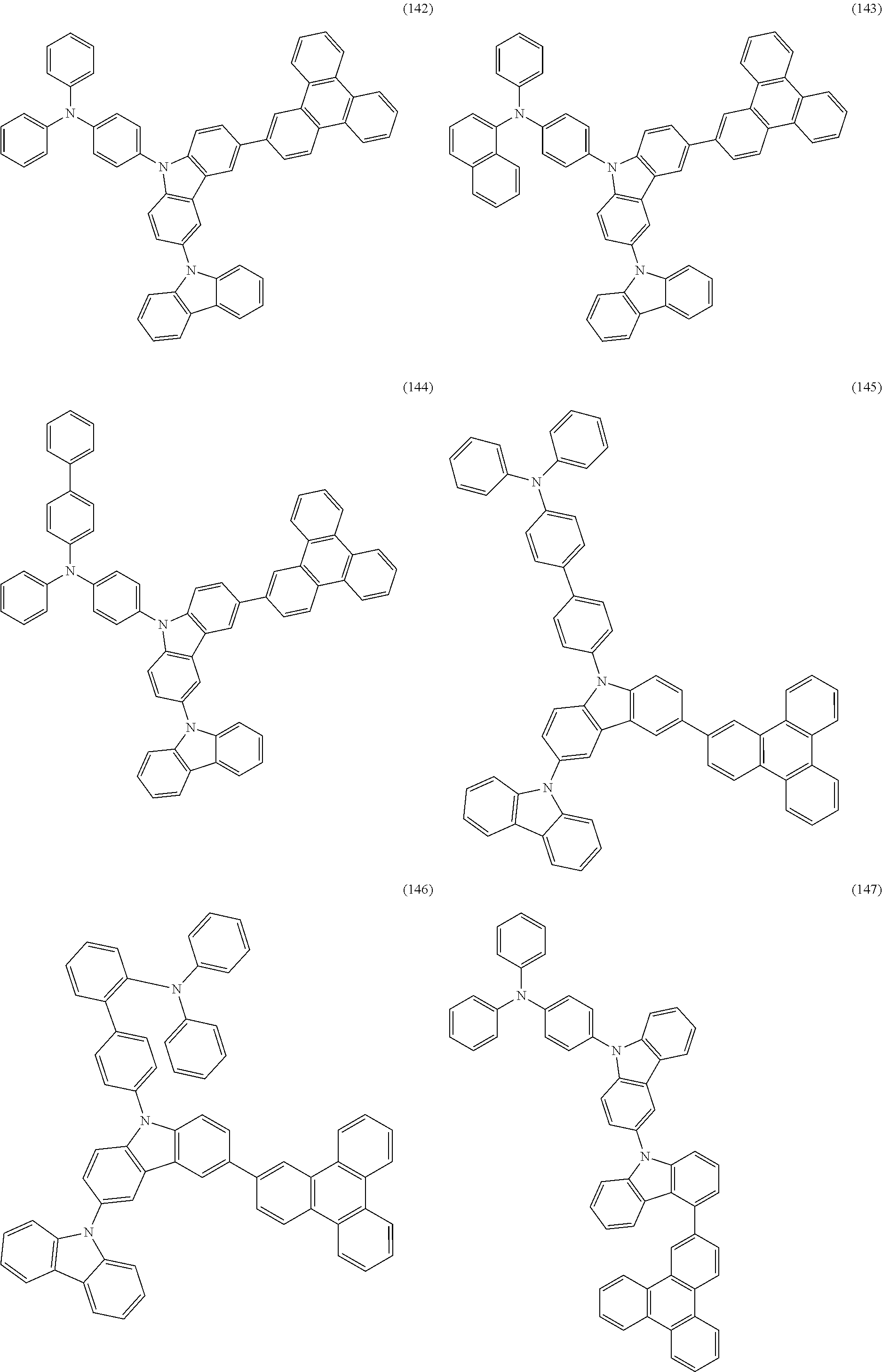

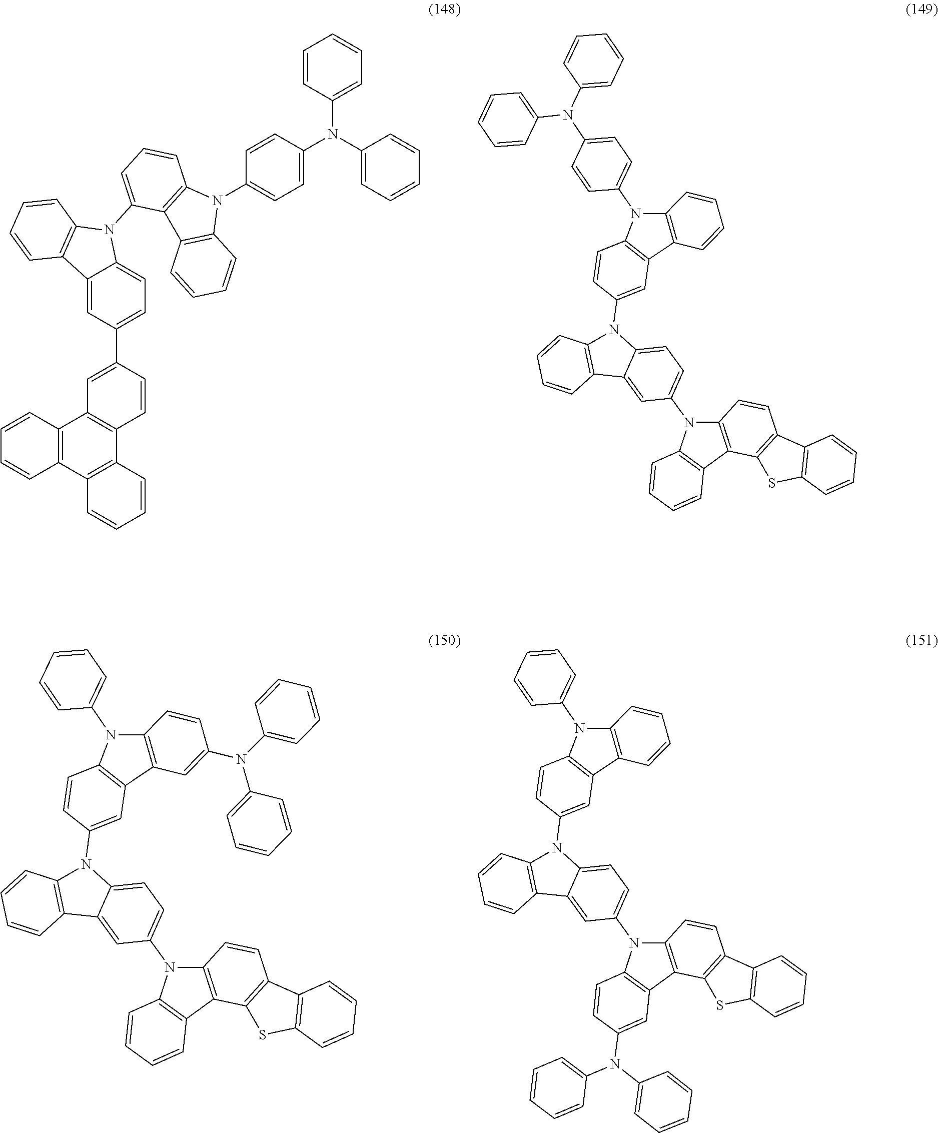

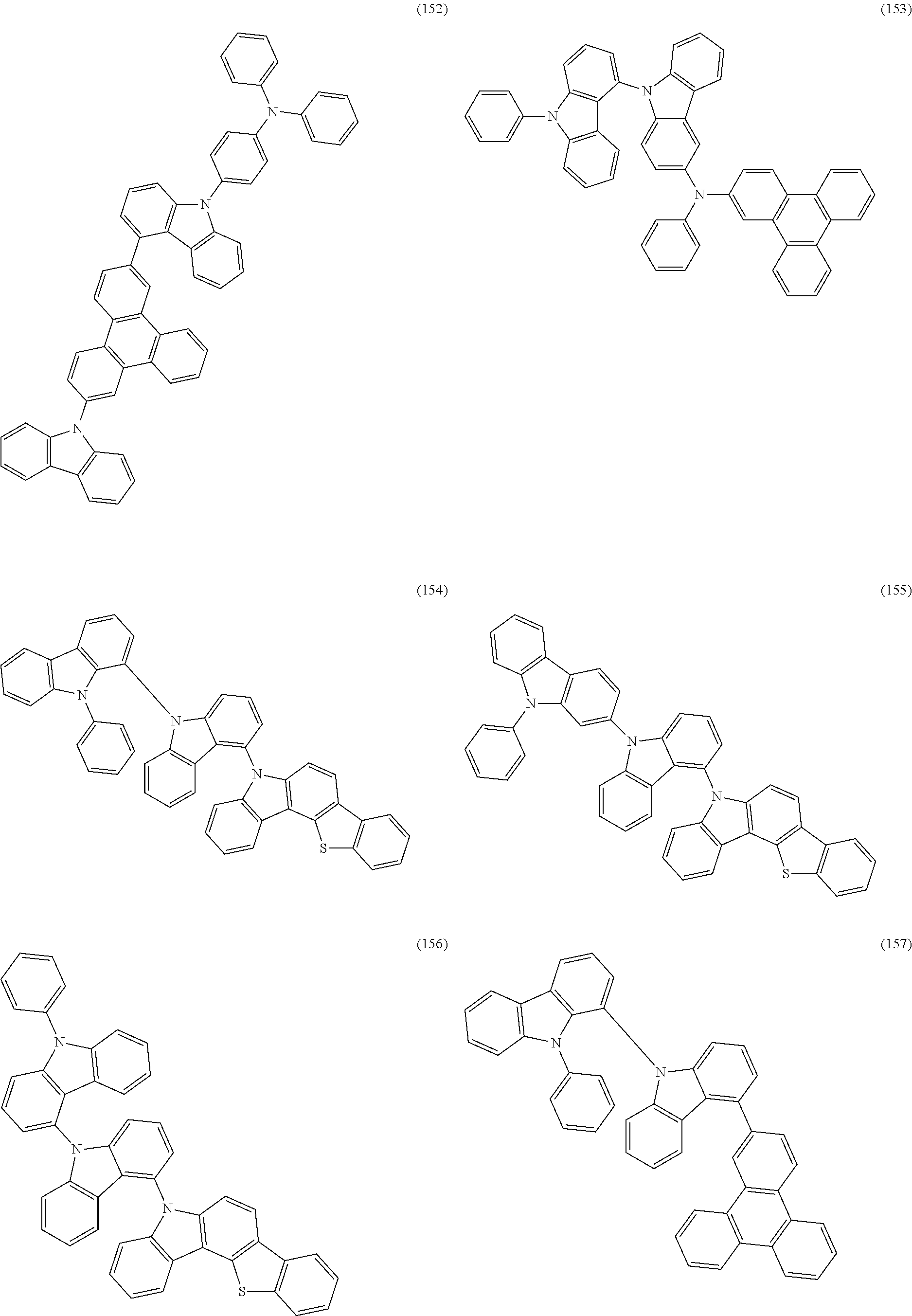

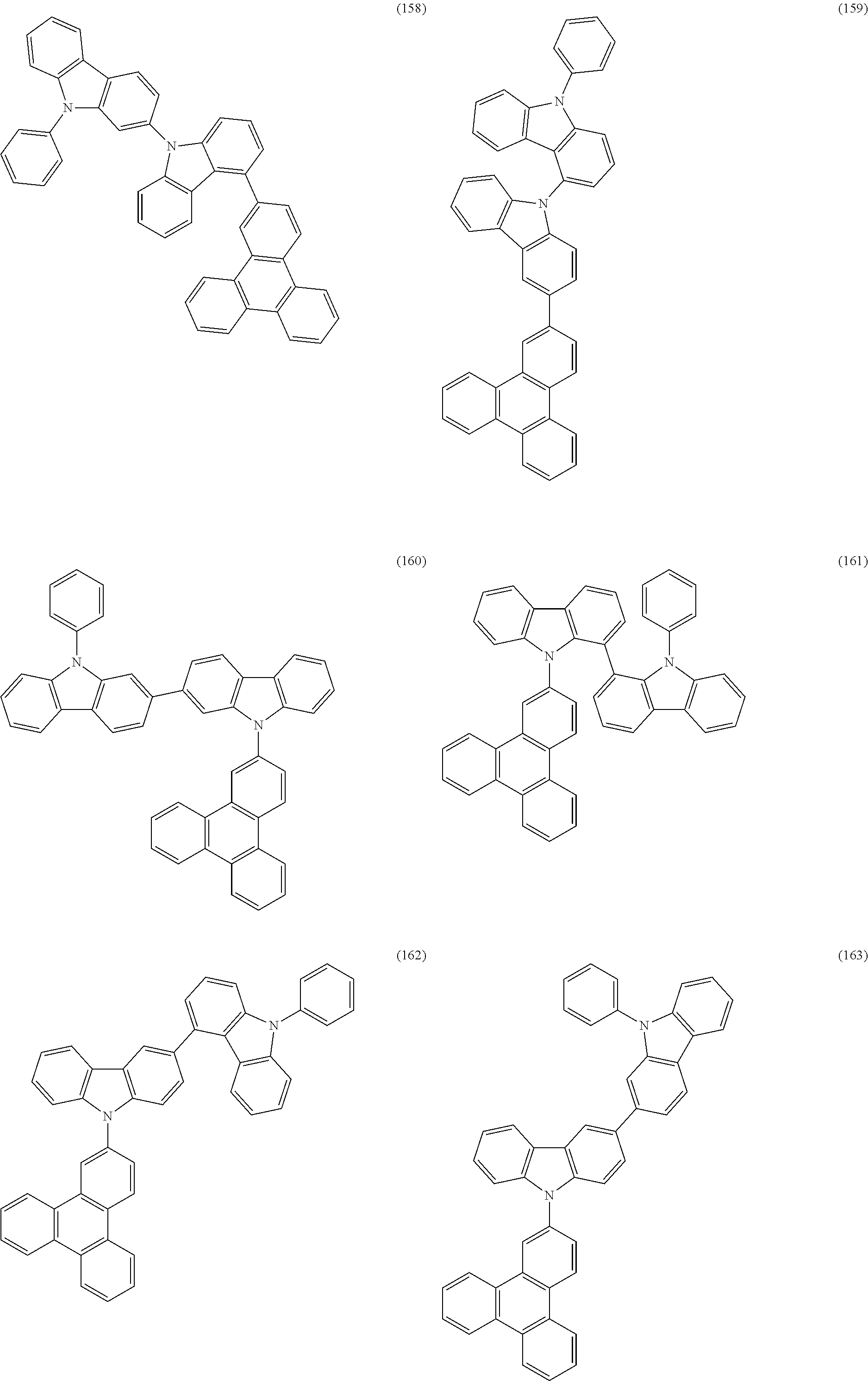

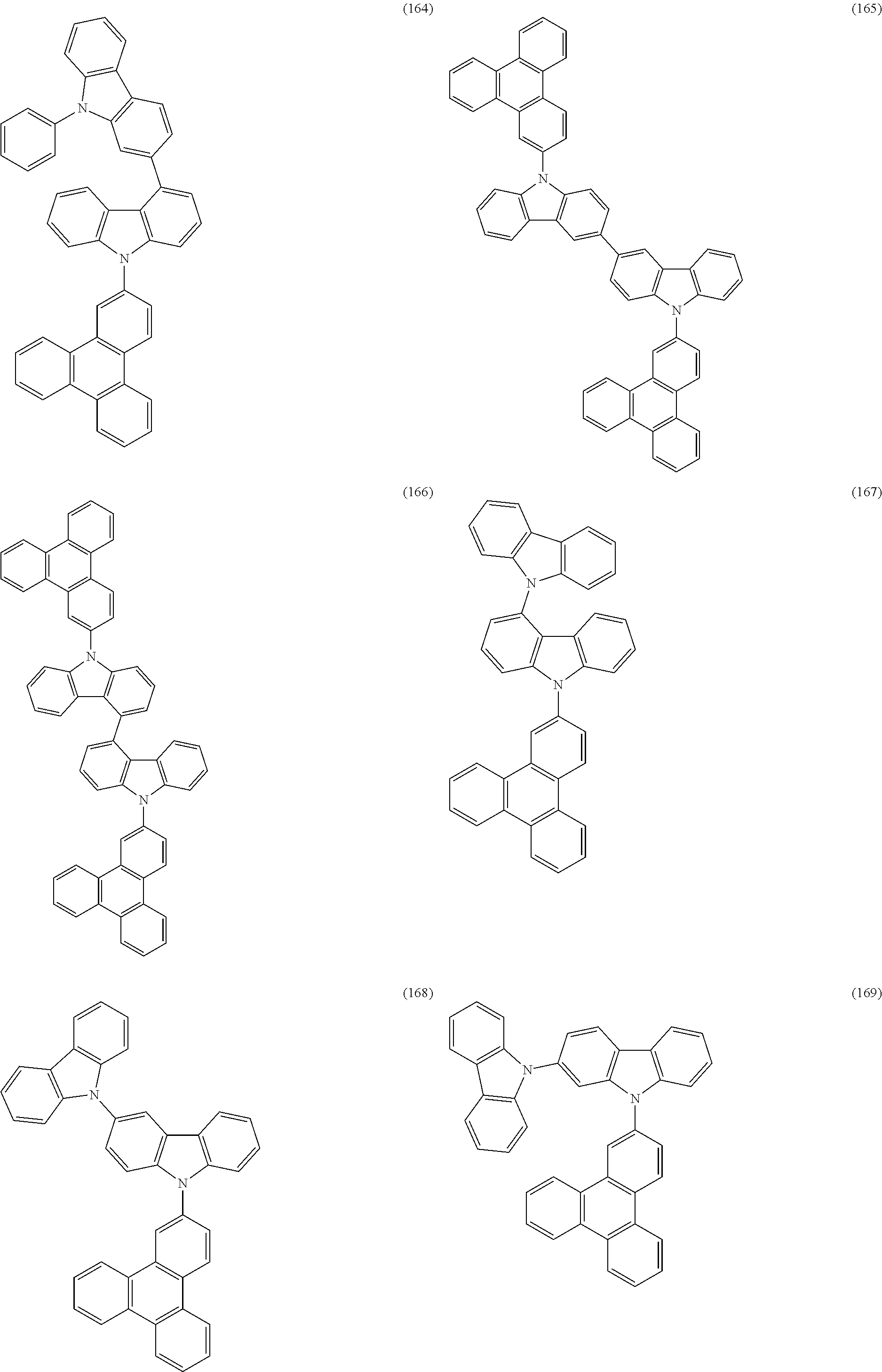

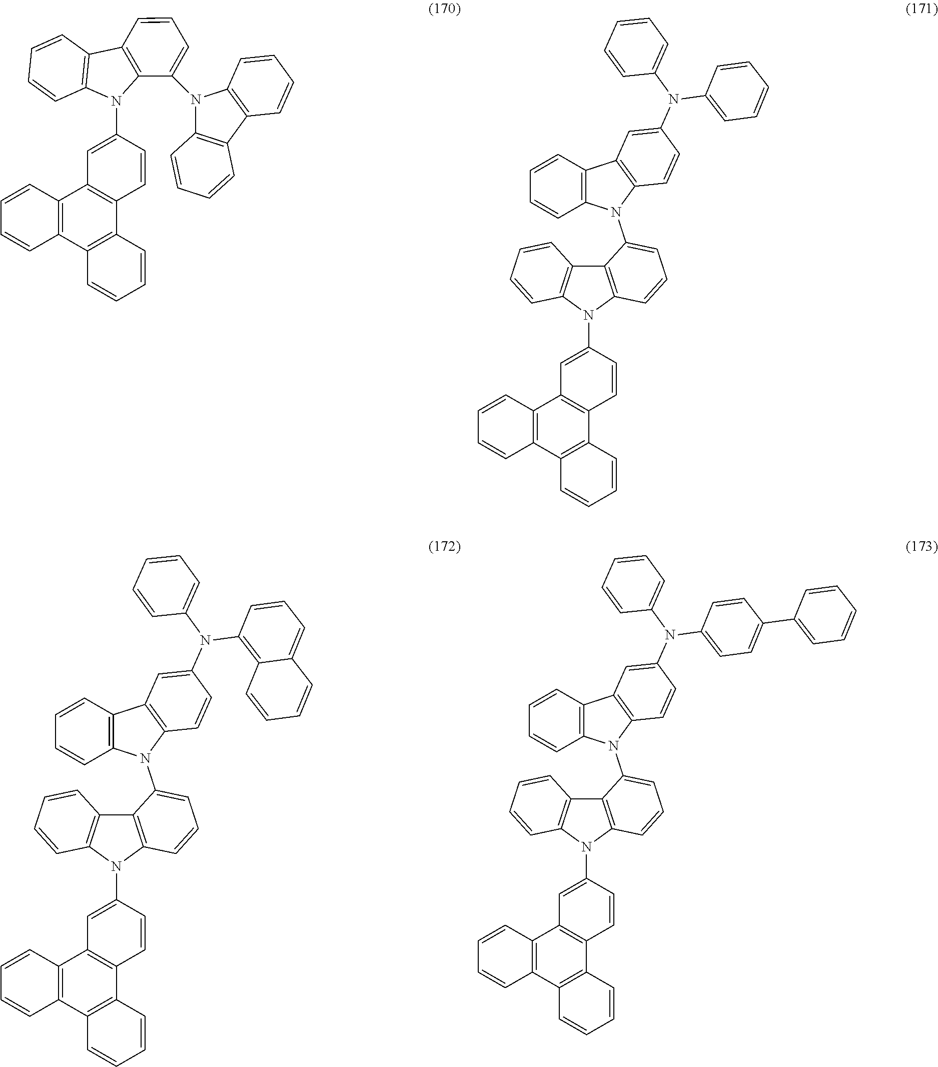

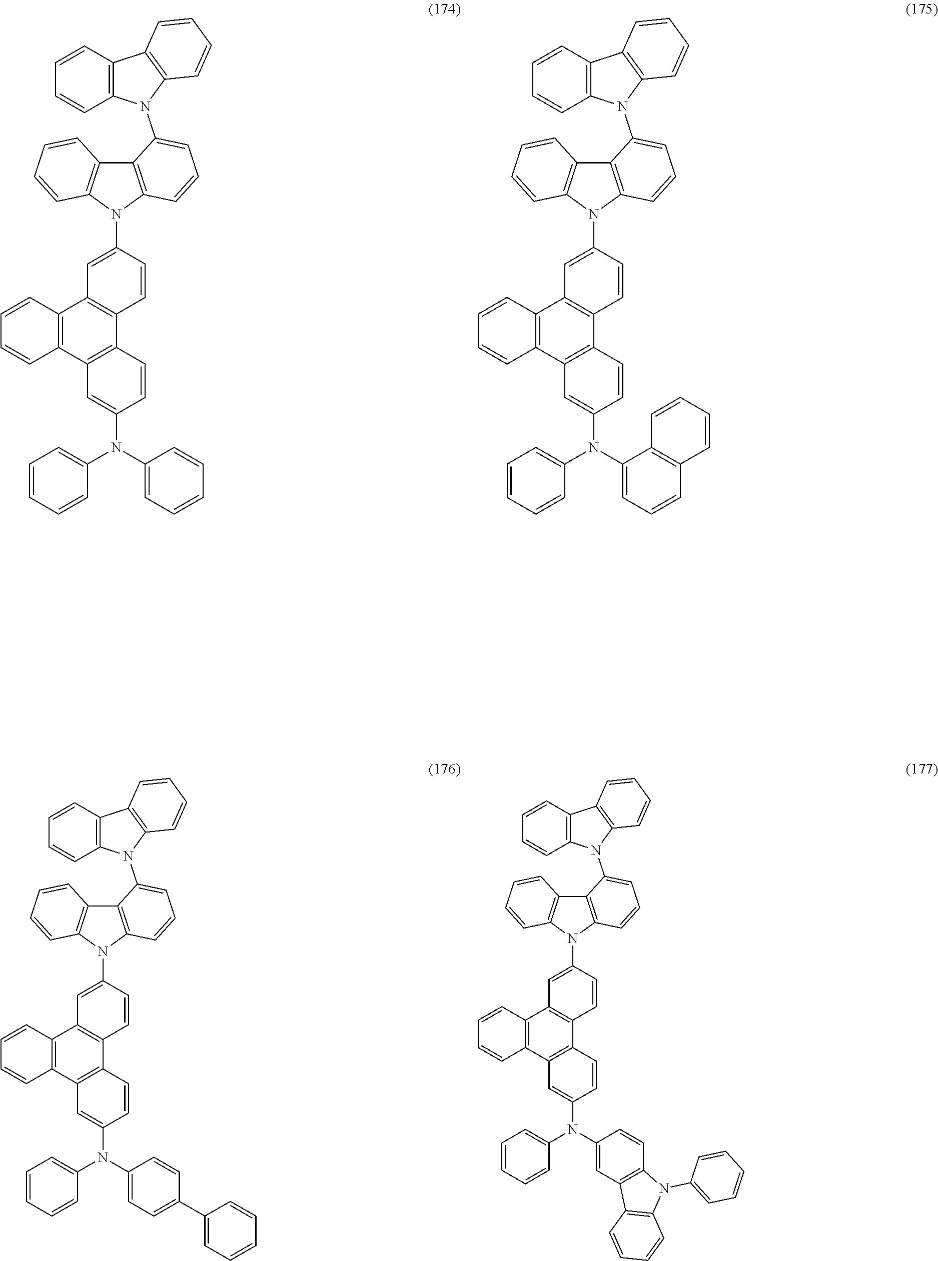

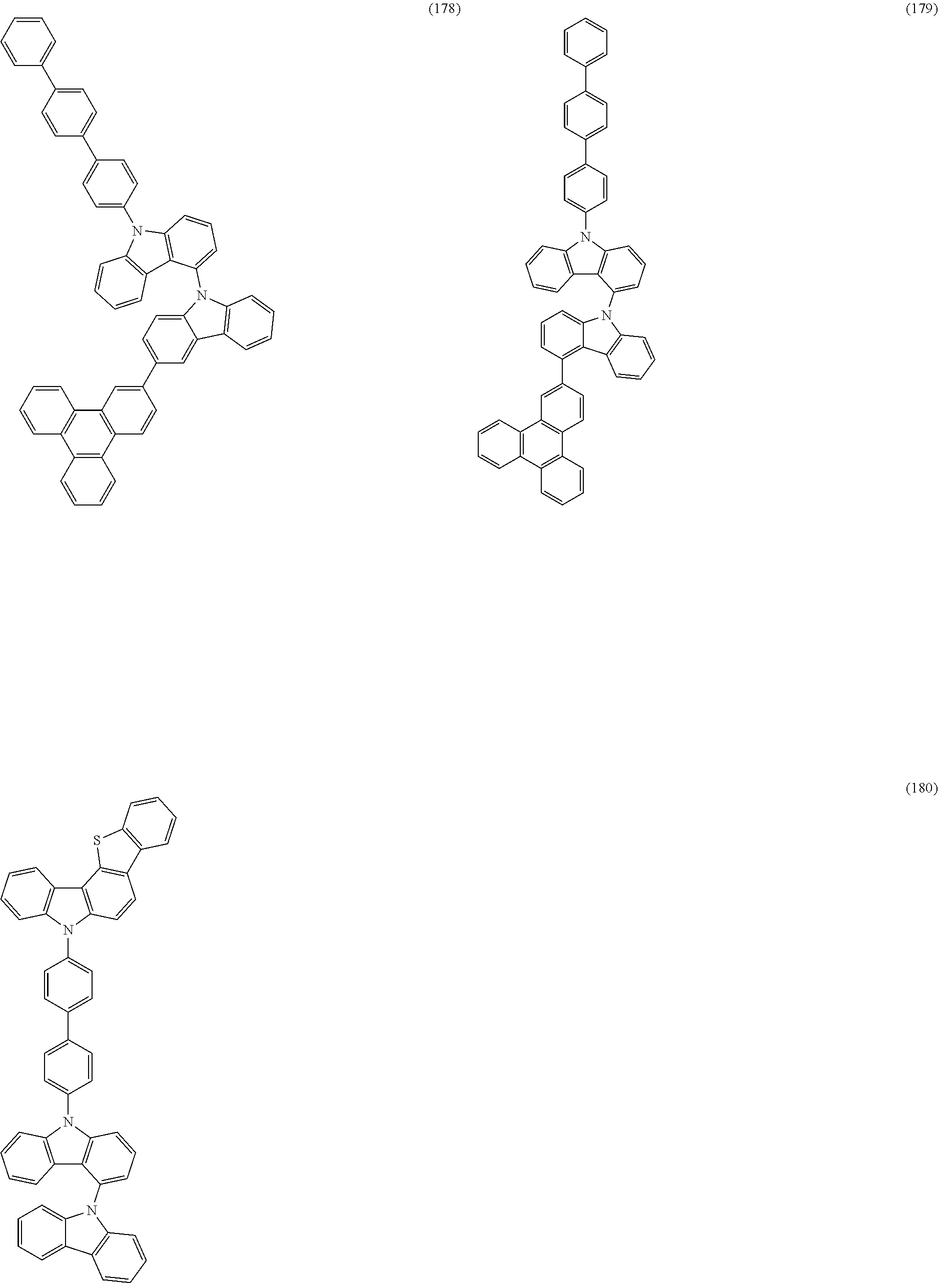

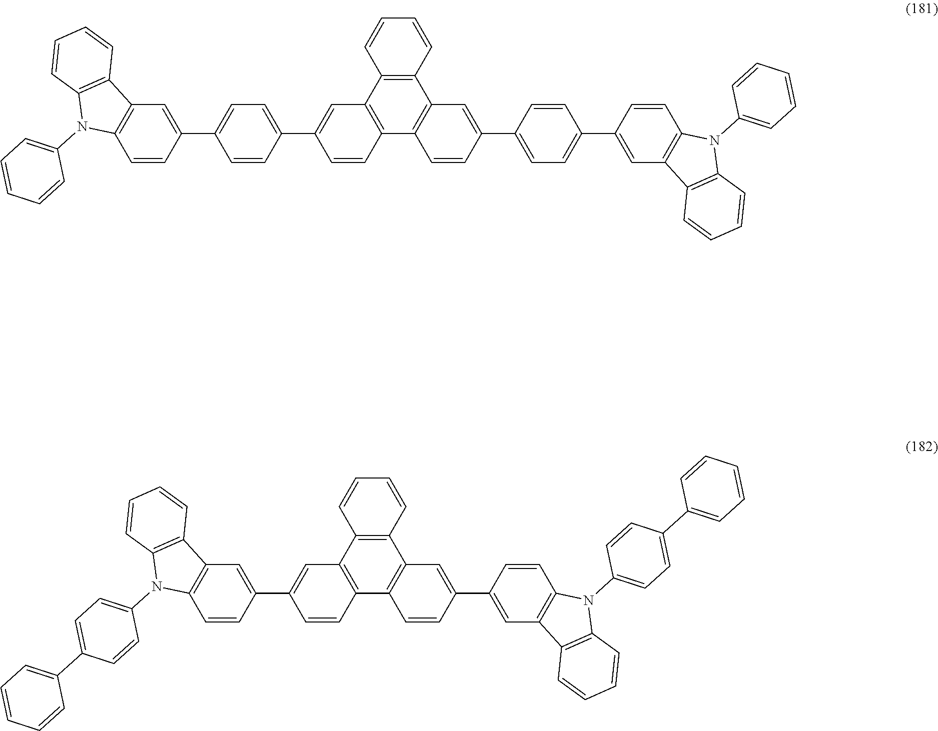

The material for a photoelectric conversion element of the present invention can be obtained by: synthesis by methods of various organic synthetic reactions established in the field of the organic synthetic chemistry including coupling reactions such as Suzuki coupling, Stille coupling, Grignard coupling, Ullmann coupling, Buchwald-Hartwig reaction, and Heck reaction, using commercially available reagents as raw materials; and then purification by using a known method such as recrystallization, column chromatography, and sublimation and purification. The method is not limited to this method.

The material for a photoelectric conversion element of the present invention preferably has an energy level of highest occupied molecular orbital (HOMO) obtained by structural optimization calculation with a density functional calculation B3LYP/6-31G (d) of −4.5 eV or lower, more preferably within a range of −4.5 eV to −6.0 eV.

The material for a photoelectric conversion element of the present invention preferably has an energy level of lowest unoccupied molecular orbital (LUMO) obtained by structural optimization calculation with a density functional calculation B3LYP/6-31G (d) of −2.5 eV or higher, more preferably within a range of −2.5 eV to −0.5 eV.

In the material for a photoelectric conversion element of the present invention, a difference (absolute value) between the HOMO energy level and the LUMO energy level is preferably within a range of 2.0 to 5.0 eV, and more preferably within a range of 2.5 to 4.0 eV.

The material for a photoelectric conversion element of the present invention preferably has a hole mobility of 1×10⁻⁶ cm²/Vs to 1 cm²/Vs, more preferably has a hole mobility of 1×10⁻⁵ cm²/Vs to 1×10⁻¹ cm²/Vs. The hole mobility can be evaluated by known methods such as a method with a FET-type transistor element, a method with a time-of-flight method, and an SCLC method.

The material for a photoelectric conversion element of the present invention is preferably amorphous. The amorphousness can be confirmed by various methods, and can be confirmed by, for example, detecting no peak in an XRD method or by detecting no endothermic peak in a DSC method.

Next, a photoelectric conversion element for imaging using the material for a photoelectric conversion element of the present invention will be described, but a structure of the photoelectric conversion element for imaging of the present invention is not limited thereto. The description will be made with reference to the Drawing.

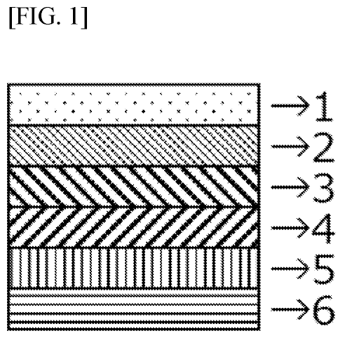

FIG. 1 is a sectional view schematically illustrating a structure of the photoelectric conversion element for imaging using the material for the photoelectric conversion element for imaging of the present invention. In FIG. 1, 1 represents a substrate, 2 represents an electrode, 3 represents an electron blocking layer, 4 represents a photoelectric conversion layer, 5 represents a hole blocking layer, and 6 represents an electrode. The photoelectric conversion element is not limited to the structure in FIG. 1, and adding or omitting a layer can be made as necessary. An inverted structure of FIG. 1, that is, an electrode 6, a hole blocking layer 5, a photoelectric conversion layer 4, an electron blocking layer 3, and an electrode 2 may be stacked on a substrate 1 in this order. In this case, adding or omitting a layer can also be made as necessary. In the photoelectric conversion element for imaging as noted above, the layers constituting the stacked structure on the substrate other than electrodes, such as a positive electrode and a negative electrode, may be collectively referred to as an organic layer.

### —Electrode—

An electrode used for the photoelectric conversion element for imaging of the present invention has a function of trapping a hole and an electron generated in the photoelectric conversion layer. A function to let light enter the photoelectric conversion layer is also required. Thus, at least one of two electrodes is desirably transparent or semi-transparent. A material used for the electrode is not particularly limited as long as it has conductivity, and examples thereof include: conductive transparent materials, such as ITO, IZO, SnO₂, ATO (antimony-doped tin oxide), ZnO, AZO (Al-doped zinc oxide), GZO (gallium-doped zinc oxide), TiO₂, and FTO; metals, such as gold, silver, platinum, chromium, aluminum, iron, cobalt, nickel, and tungsten; inorganic conductive substances, such as copper iodide and copper sulfide; and conductive polymers, such as polythiophene, polypyrrole, and polyaniline. A plurality of these materials may be mixed to use as necessary. In addition, two or more layers thereof may be stacked.

### —Photoelectric Conversion Layer—

The photoelectric conversion layer is a layer in which a hole and an electrode are generated by charge separation of an exciton generated by the incident light. The photoelectric conversion layer may be formed with a single photoelectric converting material, or may be formed by combination with a P-type organic semiconductor material being a hole transport material and an N-type organic semiconductor material being an electron transport material. Two or more kinds of the P-type organic semiconductor may be used, and two or more kinds of the N-type organic semiconductor may be used. One or more kinds of these P-type organic semiconductor and/or N-type organic semiconductor desirably use a dye material having a function of absorbing light with a desired wavelength in the visible region. As the P-type organic semiconductor material being the hole transport material, the material for a photoelectric conversion element of the present invention can be used.

The P-type organic semiconductor material may be any material having a hole transportability. The material for a photoelectric conversion element of the present invention is preferably used, but another P-type organic semiconductor material may be used. In addition, two or more kinds of the compounds represented by formulae (1) to (3) may be mixed to use. Furthermore, the above compound and another P-type organic semiconductor material may be mixed to use.

The another P-type organic semiconductor material may be any material having the hole transportability, and examples thereof include: compounds having a fused polycyclic aromatic group such as naphthalene, anthracene, phenanthrene, pyrene, chrysene, naphthacene, triphenylene, perylene, fluoranthene, fluorene, and indene; compounds having a T-excess aromatic group such as a cyclopentadiene derivative, a furan derivative, a thiophene derivative, a pyrrole derivative, a benzofuran derivative, a dibenzothiophene derivative, a dinaphthothienothiophene derivative, an indole derivative, a pyrazoline derivative, a dibenzofuran derivative, a dibenzothiophene derivative, a carbazole derivative, and indolocarbazole; an aromatic amine derivative, a styrylamine derivative, a benzidine derivative, a porphyrin derivative, a phthalocyanine derivative, and a quinacridone derivative.

In addition, examples of a polymer P-type organic semiconductor material include a polyphenylene-vinylene derivative, a polyparaphenylene derivative, a polyfluorene derivative, a polyvinylcarbazole derivative, and a polythiophene derivative. Two or more kinds selected from the compounds represented by the formulae (1) to (3) of the present invention, the P-type organic semiconductor material, and the polymer P-type organic semiconductor material may be mixed to use.

The N-type organic semiconductor material may be any material having the electron transportability, and examples thereof include naphthalenetetracarboxylic diimide and perylenetetracarboxylic diimide, fullerenes, and azole derivatives such as imidazole, thiazole, thiadiazole, oxazole, oxadiazole, and triazole. Two or more kinds selected from the N-type organic semiconductor materials may be mixed to use.

### —Electron Blocking Layer—

The electron blocking layer is provided in order to inhibit a dark current generated by injecting an electron from one electrode into the photoelectric conversion layer when a bias voltage is applied between the two electrodes. The electron blocking layer also has a function of hole transportation for transporting a hole generated by charge separation in the photoelectric conversion layer toward the electrode. A single layer or multiple layers of the electron blocking layer can be disposed as necessary. For the electron blocking layer, a P-type organic semiconductor material being the hole transport material can be used. The P-type organic semiconductor material may be any material having the hole transportability. Although the compound represented by the formulae (1) to (3) is preferably used, another P-type organic semiconductor material may be used. In addition, the compound represented by the formulae (1) to (3) and the other P-type organic semiconductor material as noted above or a polymer P-type organic semiconductor material may be mixed to use.

### —Hole Blocking Layer—

The hole blocking layer is provided in order to inhibit a dark current generated by injecting a hole from one electrode into the photoelectric conversion layer when a bias voltage is applied between the two electrodes. The hole blocking layer also has a function of electron transportation for transporting an electron generated by charge separation in the photoelectric conversion layer toward the electrode. A single layer or multiple layers of the hole blocking layer can be disposed as necessary. For the hole blocking layer, the N-type organic semiconductor material having the electron transportability can be used. The N-type organic semiconductor material may be any material having the electron transportability, and examples thereof include: polycyclic aromatic multivalent carboxylic anhydride or imidized products thereof, such as naphthalenetetracarboxylic diimide and perylenetetracarboxylic diimide; fullerenes, such as C60 and C70; azole derivatives, such as imidazole, thiazole, thiadiazole, oxazole, oxadiazole, and triazole; a tris(8-quinolinolate)aluminum (III) derivative, a phosphine oxide derivative, a nitro-substituted fluorene derivative, a diphenylquinone derivative, a thiopyran dioxide derivative, a carbodiimide, a fluorenylidene methane derivative, an anthraquinodimethane derivative and an anthrone derivative, a bipyridine derivative, a quinoline derivative, and an indolocarbazole derivative. Two or more kinds selected from the N-type organic semiconductor materials may be mixed to use.

Hydrogen in the material of the present invention may be deuterium. That is, a part or all of hydrogens on the aromatic rings in the general formulae (1) to (5), and hydrogen on the aromatic rings of Ar¹ to Ar⁶, L¹, and R may be deuterium. Furthermore, a part or all of hydrogens in a compound used as the N-type organic semiconductor material and the P-type organic semiconductor material may be deuterium.

A method for producing a film of each layer in producing the photoelectric conversion element for imaging of the present invention is not particularly limited. The photoelectric conversion element may be produced by any one of dry process and wet process. The organic layer containing the material for a photoelectric conversion element of the present invention may be a plurality of layers as necessary.

### EXAMPLES

Hereinafter, the present invention will be described in more detail with Examples, but the present invention is not limited to these Examples.

### Calculation Example

### Calculation of HOMO and LUMO Values

Calculated were HOMO and LUMO of the compounds shown in the following Table 1 of the above compounds. The calculation was performed by using a density functional theory (DFT), using Gaussian as a calculation program, and with structural optimization calculation of a density functional calculation B3LYP/6-31G (d). Table 1 shows the results. It can be mentioned that any of the materials for the photoelectric conversion element for imaging of the present invention has preferable HOMO and LUMO values.

As comparison, HOMO and LUMO for the compounds H1 and H2 were calculated by the same method. Table 1 shows the results.

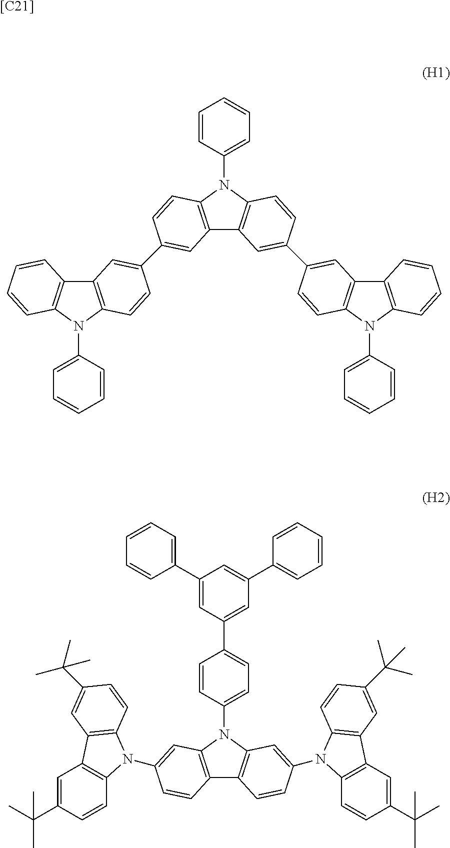

TABLE 1

| Compound | HOMO[eV] | LUMO[eV] |
|---|---|---|
| 1 | −5.0 | −1.1 |
| 7 | −5.0 | −1.2 |
| 33 | −5.1 | −1.0 |
| 51 | −5.2 | −1.0 |
| 60 | −5.0 | −1.1 |
| 61 | −5.0 | −1.1 |
| 62 | −5.1 | −1.1 |
| 66 | −5.0 | −1.2 |
| 93 | −5.1 | −1.1 |
| 98 | −5.3 | −1.4 |
| 118 | −5.0 | −1.3 |
| 148 | −4.9 | −1.0 |
| 159 | −5.1 | −1.0 |
| 167 | −5.2 | −1.3 |
| 178 | −5.1 | −1.0 |
| 181 | −5.1 | −1.0 |
| 182 | −5.1 | −1.0 |
| H1 | −4.9 | −0.7 |
| H2 | −5.0 | −1.3 |

Synthesis examples of the compounds 1, 33, 60, and 93 will be described below as representative examples. The other compounds were also synthesized by similar methods.

### Synthesis Example 1 (Synthesis of Compound 1)

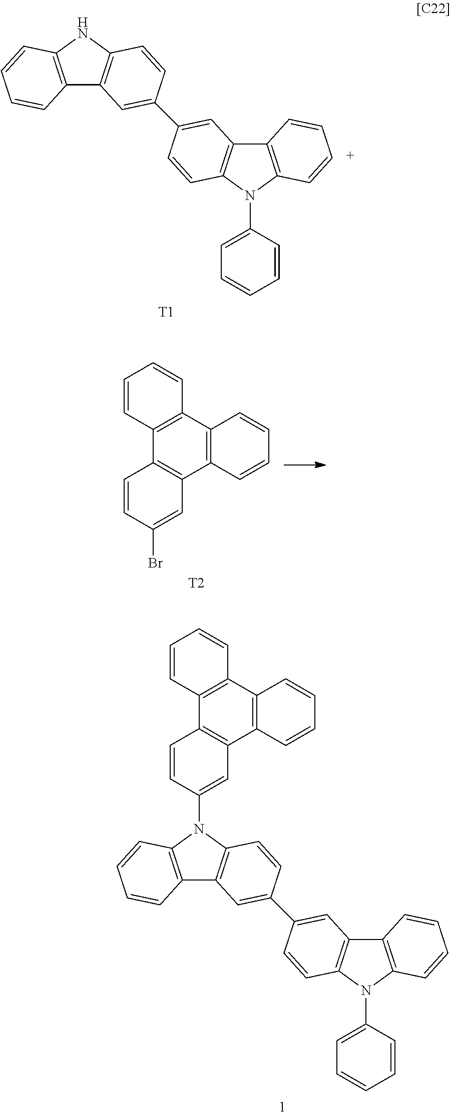

Into a three-necked 200-ml flask with degassed and nitrogen-replenished, T1 (17.2 mmol), T2 (20.6 mmol), copper iodide (5.1 mmol), potassium carbonate (51.5 mmol), and 8-quinolinol (5.1 mmol) were added, 43 ml of 1,3-dimethyl-2-imidazolidinone (DMI) was added thereinto, and then the mixture was stirred at 190° C. for 8 hours. The mixture was once cooled to a room temperature, and then 200 ml of water was added to filter a produced white precipitate. The obtained residue was purified by column chromatography to obtain a compound 1 (white solid). The obtained solid was evaluated by an XRD method but no peak was detected. Thus, this compound was found to be amorphous.

### Synthesis Example 2 (Synthesis of Compound 33)

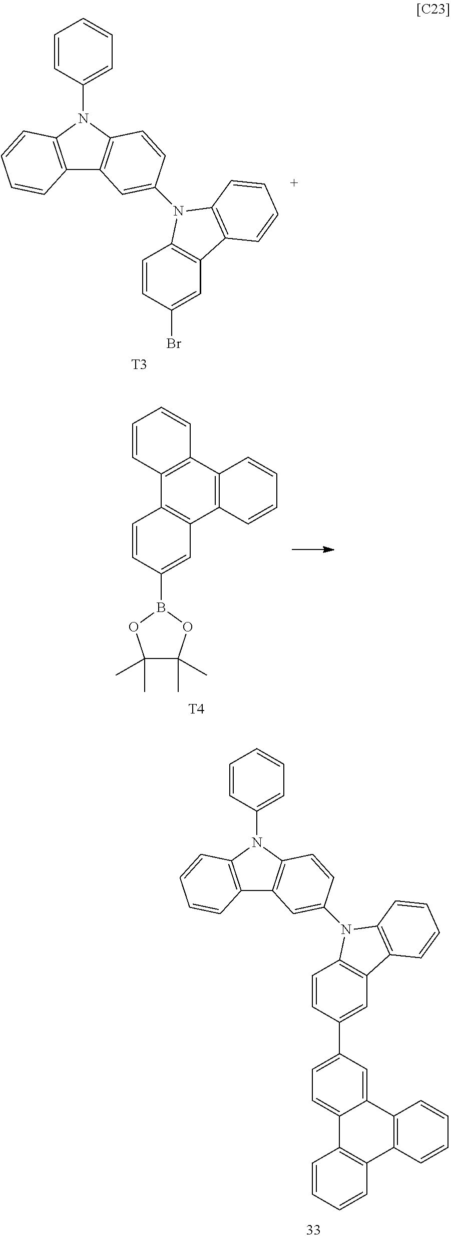

Into a three-necked 200-ml flask with degassed and nitrogen-replenished, T3 (8.5 mmol), T4 (9.3 mmol), tetrakis(triphenylphosphine) palladium (0) (0.4 mmol), and potassium carbonate (42.4 mmol) were added, and 80 ml of toluene, 20 ml of ethanol, and 20 ml of water were added thereinto, and then the mixture was stirred at 100° C. for 4 hours. The mixture was once cooled to a room temperature, then 100 ml of water was added, the mixture was transferred into a separatory funnel and separation into an organic layer and an aqueous layer was performed. The organic layer was washed three times with 100 ml of water, and then the obtained organic layer was concentrated under a reduced pressure. The obtained residue was purified by column chromatography to obtain a compound 33 (white solid). The obtained solid was evaluated by an XRD method but no peak was detected.

### Synthesis Example 3 (Synthesis of Compound 60)

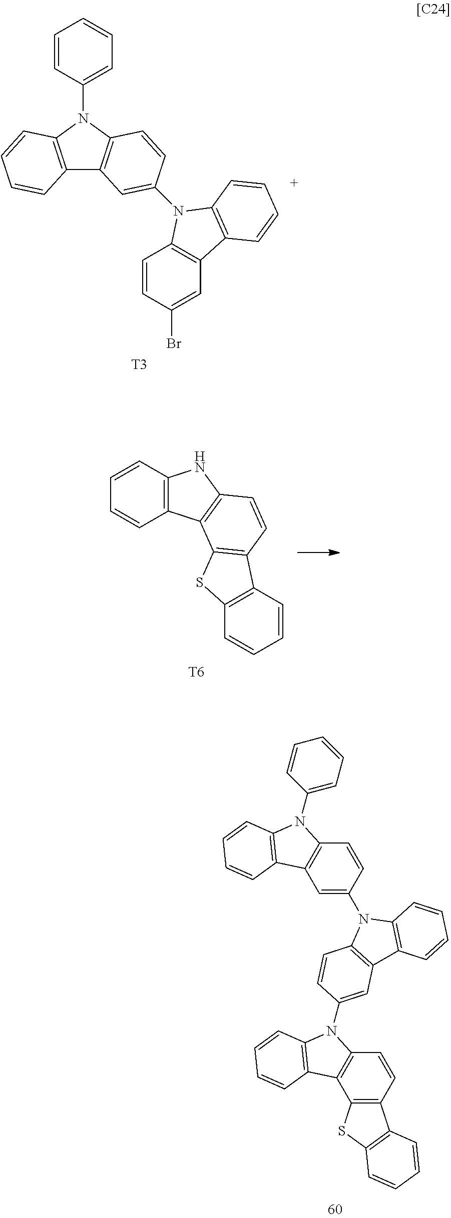 [SMILES: Brc1ccc2c(c1)c1ccccc1n2-c1ccc2c(c1)c1ccccc1n2-c1ccccc1 ; "T3" ; c1ccc2c(c1)[nH]c1ccc3c4ccccc4sc3c12 ; "T6" ; c1ccc(-n2c3ccccc3c3cc(-n4c5ccccc5c5cc(-n6c7ccccc7c7c8sc9ccccc9c8ccc76)ccc54)ccc32)cc1]

Into a three-necked 200-ml flask with degassed and nitrogen-replenished, T3 (10.1 mmol), T6 (9.2 mmol), copper iodide (2.7 mmol), potassium carbonate (27.5 mmol), and 8-quinolinol (2.7 mmol) were added, 23 ml of 1,3-dimethyl-2-imidazolidinone (DMI) was added thereinto, and then the mixture was stirred at 190° C. for 8 hours. The mixture was once cooled to a room temperature, and then 100 ml of water was added to filter a produced white precipitate. The obtained residue was purified by column chromatography to obtain a compound 60 (white solid). The obtained white solid was evaluated by an XRD method but no peak was detected.

### Synthesis Example 4 (Synthesis of Compound 93)

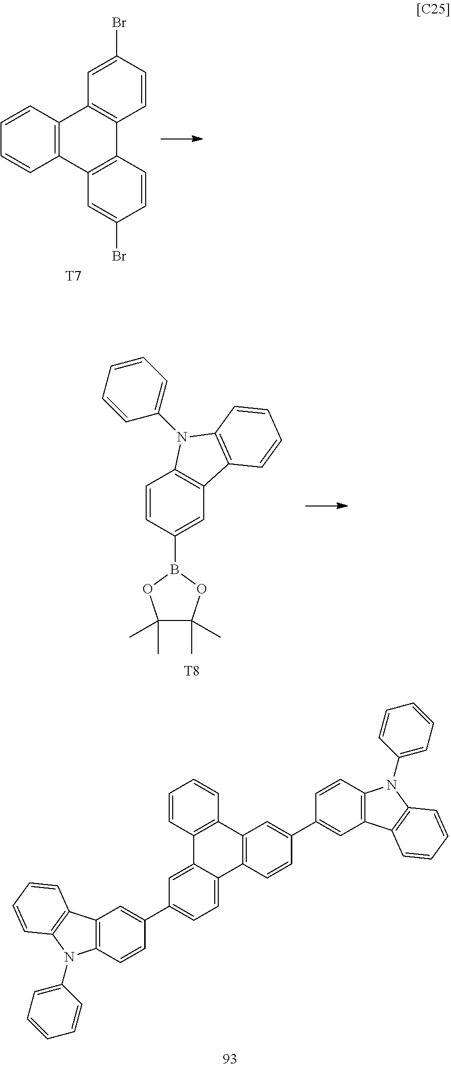

Into a three-necked 200-ml flask with degassed and nitrogen-replenished, T7 (9.9 mmol), T8 (21.7 mmol), tetrakis(triphenylphosphine) palladium (0) (0.5 mmol), and potassium carbonate (49.3 mmol) were added, 220 ml of toluene, 55 ml of ethanol, and 55 ml of water were added thereinto, and then the mixture was stirred at 100° C. for 4 hours. The mixture was once cooled to a room temperature, then 200 ml of water was added, the mixture was transferred into a separatory funnel and separation into an organic layer and an aqueous layer was performed. The organic layer was washed three times with 200 ml of water, and then the obtained organic layer was concentrated under a reduced pressure. The obtained residue was purified by column chromatography to obtain the compound 93 (white solid). The obtained solid was evaluated by an XRD method but no peak was detected.

### Example of Physical Properties Evaluation

On a glass substrate on which a transparent electrode composed of ITO with 110 nm in film thickness was formed, the compound 1 was produced to a film as an organic layer by a vacuum deposition method under a condition that a film thickness was approximately 3 μm. Subsequently, charge mobility was measured by a time-of-flight method using an element in which aluminum (Al) was formed with 70 nm in thickness as an electrode. As a result, the hole mobility was 8.1×10⁻⁵ cm²/Vs.

The hole mobility was evaluated in the same procedure except that compounds shown in the following Table 2 were used instead of the compound 1. Table 2 shows the results.

TABLE 2

| Compound | Hole mobility [cm²/Vs] |
|---|---|
| 1 | 8.1 × 10⁻⁵ |
| 33 | 1.9 × 10⁻⁴ |
| 51 | 4.5 × 10⁻⁵ |
| 60 | 6.5 × 10⁻⁵ |
| 61 | 1.1 × 10⁻⁵ |
| 62 | 5.3 × 10⁻⁵ |
| 93 | 7.2 × 10⁻⁵ |
| 118 | 8.7 × 10⁻⁵ |
| 148 | 2.3 × 10⁻⁴ |
| 159 | 1.0 × 10⁻⁵ |
| 167 | 4.5 × 10⁻⁵ |
| 181 | 1.2 × 10⁻⁵ |
| 182 | 1.3 × 10⁻⁵ |
| H1 | 1.2 × 10⁻⁵ |
| H2 | 9.3 × 10⁻⁶ |

### Example 1

On a glass substrate on which an electrode composed of ITO with 70 nm in film thickness was formed, a 100-nm film of the compound 93 was formed with a vacuum degree of 4.0×10⁻⁵ Pa as an electron blocking layer. Then, a 100-nm thin film of quinacridone was formed as a photoelectric conversion layer. Finally, a 70-nm aluminum film was formed as an electrode to produce a photoelectric conversion element. A current in a dark place was 2.5×10⁻¹⁰ A/cm² with the electrodes of ITO and aluminum and with applying a voltage of 2 V. When a voltage of 2 V was applied on the ITO electrode (transparent conductive glass) side and the side was irradiated with light to be an irradiation light wavelength of 500 nm, a current was 1.4×10⁻⁷ A/cm². A contrast ratio with applying a voltage of 2 V on the transparent conductive glass side was 5.6×10².

### Comparative Example 1

On a glass substrate on which an electrode composed of ITO with 70 nm in film thickness was formed, a 100-nm film of the compound H1 was formed with a vacuum degree of 4.0×10⁻⁵ Pa as an electron blocking layer. Then, a 100-nm film of quinacridone was formed as a photoelectric conversion layer. Finally, a 70-nm aluminum film was formed as an electrode to produce a photoelectric conversion element. A current in a dark place was 5.6×10⁻⁹ A/cm² with the electrodes of ITO and aluminum and with applying a voltage of 2 V. When a voltage of 2 V was applied on the ITO electrode side and the side was irradiated with light to be an irradiation light wavelength of 500 nm, a current was 1.2×10⁻⁷ A/cm². A contrast ratio with applying a voltage of 2 V on the transparent conductive glass side was 0.21×10².

### Example 2

On an electrode composed of ITO with 70 nm in film thickness and formed on a glass substrate, a 10-nm film of the compound 1 was formed with a vacuum degree of 4.0×10⁻⁵ Pa as an electron blocking layer. Then, 2Ph-BTBT, F6-SubPc-OC6F5, and fullerene (C60) were co-deposited at a deposition rate ratio of 4:4:2 with 200 nm to form a film as a photoelectric conversion layer. Subsequently, 10-nm of dpy-NDI was deposited to form a hole blocking layer. Finally, an aluminum film was formed with 70 nm in thickness as an electrode to produce a photoelectric conversion element. A current in a dark place (dark current) was 6.3×10⁻¹⁰ A/cm² with the electrodes of ITO and aluminum and with applying a voltage of 2.6 V. When a voltage of 2.6 V was applied and the ITO electrode side was irradiated with light with an LED adjusted to be an irradiation light wavelength of 500 nm and 1.6 μW from a height of 10 cm, a current (bright current) was 3.5×10⁻⁷ A/cm². A contrast ratio was 5.6×10² with applying a voltage of 2.6 V. Table 3 shows the results.

### Examples 3 to 13

Photoelectric conversion elements were produced in the same manner as in Example 2 except that compounds shown in Table 3 were used as the electron blocking layer.

### Comparative Examples 2 to 3

Photoelectric conversion elements were produced in the same manner as in Example 2 except that compounds shown in Table 3 were used as the electron blocking layer.

Table 3 shows the results of Examples 2 to 13 and Comparative Examples 2 to 3.

The compounds used in Examples and Comparative Examples are shown below.

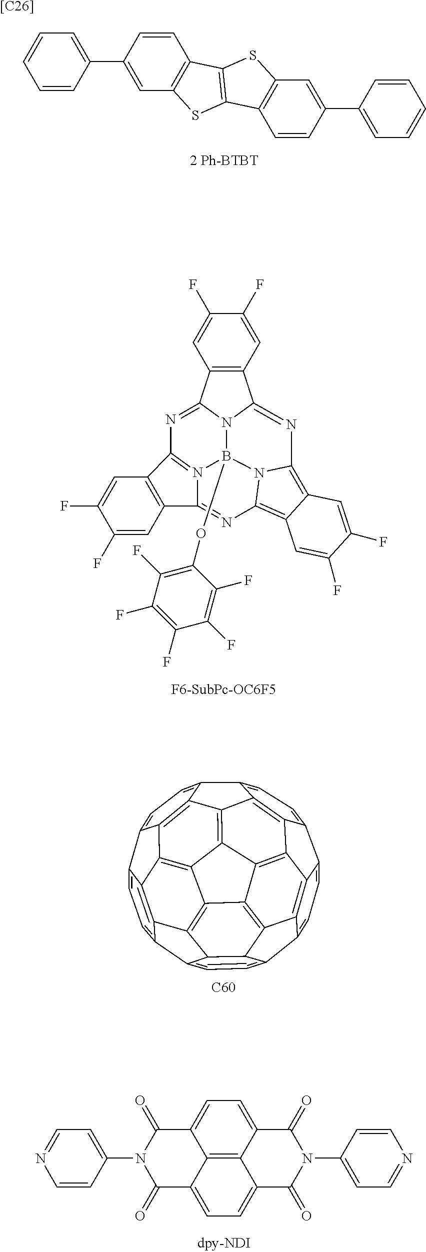

TABLE 3

| | | | Current value | |
|---|---|---|---|---|
| | | Current value | in light | |
| | | in dark place | irradiation | Contrast |
| | Compound | [A/cm²] | [A/cm²] | ratio |
| Example 2 | 1 | 6.3 × 10⁻¹⁰ | 3.5 × 10⁻⁷ | 5.6 × 10² |
| Example 3 | 33 | 5.1 × 10⁻¹⁰ | 3.1 × 10⁻⁷ | 6.1 × 10² |
| Example 4 | 51 | 3.9 × 10⁻¹⁰ | 3.1 × 10⁻⁷ | 7.9 × 10² |
| Example 5 | 60 | 5.3 × 10⁻¹⁰ | 3.4 × 10⁻⁷ | 6.4 × 10² |
| Example 6 | 61 | 4.3 × 10⁻¹⁰ | 3.4 × 10⁻⁷ | 7.9 × 10² |
| Example 7 | 62 | 4.9 × 10⁻¹⁰ | 3.3 × 10⁻⁷ | 6.7 × 10² |
| Example 8 | 93 | 4.5 × 10⁻¹⁰ | 3.1 × 10⁻⁷ | 6.9 × 10² |
| Example 9 | 148 | 5.1 × 10⁻¹⁰ | 3.3 × 10⁻⁷ | 6.5 × 10² |
| Example 10 | 159 | 4.4 × 10⁻¹⁰ | 3.2 × 10⁻⁷ | 7.3 × 10² |
| Example 11 | 167 | 4.1 × 10⁻¹⁰ | 3.0 × 10⁻⁷ | 7.3 × 10² |
| Example 12 | 181 | 3.2 × 10⁻¹⁰ | 3.0 × 10⁻⁷ | 9.4 × 10² |
| Example 13 | 182 | 4.7 × 10⁻¹⁰ | 2.9 × 10⁻⁷ | 6.9 × 10² |
| Comparative | H1 | 7.7 × 10⁻¹⁰ | 2.4 × 10⁻⁷ | 3.1 × 10² |
| Example 2 | | | | |
| Comparative | H2 | 6.1 × 10⁻¹⁰ | 2.0 × 10⁻⁷ | 3.3 × 10² |
| Example 3 | | | | |

It is found from the results in Table 3 that the photoelectric conversion elements using the compound of the present invention exhibit a low dark current value and a high contrast ratio.

### INDUSTRIAL APPLICABILITY

Using the material for a photoelectric conversion element for imaging of the present invention can achieve appropriate move of the hole and the electron in the photoelectric conversion element, and consequently enables to reduce a leakage current generated by applying a bias voltage during the conversion of light into electric energy. As a result, it is considered that a photoelectric conversion element that achieves a low dark current value and a high contrast ratio can be obtained. The material of the present invention is useful as a material for a photoelectric conversion element for a photoelectric-converting film-stacked imaging device.

### REFERENCE SIGNS LIST

1 Substrate, 2 Electrode, 3 Electron blocking layer, 4 Photoelectric conversion layer, 5 Hole blocking layer, 6 Electrode

1. A material for a photoelectric conversion element for imaging, the material comprising a carbazole compound represented by the following general formula (1), (2), or (3): 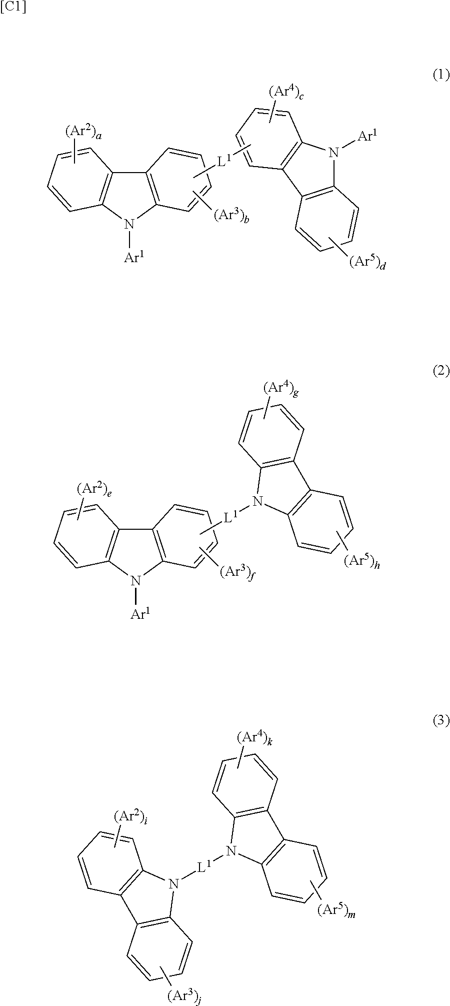
wherein in the general formulae (1), (2), and (3), Ar¹ to Ar⁵ each independently represent deuterium, a cyano group, a substituted or unsubstituted diarylamino group having 12 to 30 carbon atoms, a substituted or unsubstituted arylheteroarylamino group having 12 to 30 carbon atoms, a substituted or unsubstituted diheteroarylamino group having 12 to 30 carbon atoms, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to six of aromatic rings of the aromatic hydrocarbon group or the aromatic heterocyclic group are linked; L¹ each independently represents a direct bond, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to six of aromatic rings thereof are linked; “a” to “k” and “m” each independently represent an integer of 0 to 3; at least one of L¹ and Ar¹ to Ar⁵ represents a group having an aromatic ring structure selected from the following formula (4) or (5), and the aromatic ring structure optionally has a substituent; L¹ or Ar¹ to Ar⁵ do not represent an aromatic heterocyclic group having 12 or more carbon atoms except for a case of the group having the aromatic ring structure, 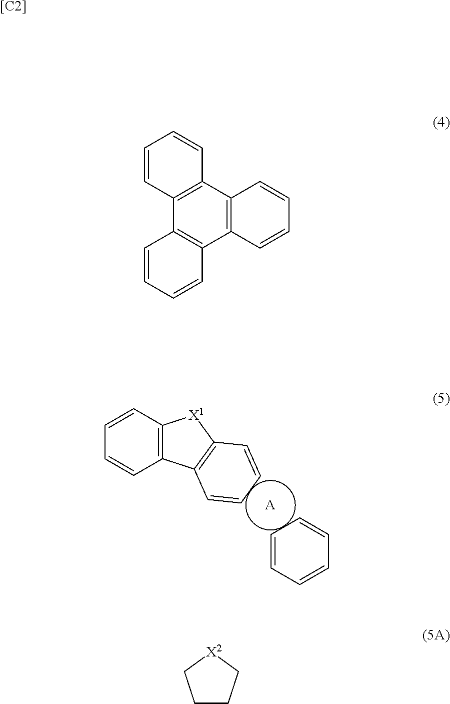
wherein a ring A represents a heterocyclic ring represented by formula (5), and the ring A is fused with an adjacent ring at any position; X¹ represents O, S, Se, N—R, or N; X² represents O, S, or Se; R represents a substituted or unsubstituted aromatic hydrocarbon group having 6 to 30 carbon atoms, a substituted or unsubstituted aromatic heterocyclic group having 3 to 11 carbon atoms, or a substituted or unsubstituted linked aromatic group in which two to six of aromatic rings thereof are linked.

2. The material for a photoelectric conversion element according to claim 1, wherein the Ar¹ to Ar⁵ each independently represents deuterium, a substituted or unsubstituted aromatic hydrocarbon group having 6 to 30 carbon atoms, a substituted or unsubstituted aromatic heterocyclic group having 3 to 18 carbon atoms, or a substituted or unsubstituted linked aromatic group in which two to six of the aromatic hydrocarbon group or the aromatic heterocyclic group are linked; “a” to “f” each represent 0; and g+h, i+j, and k+m each independently represent 0 or 1.

3. The material for a photoelectric conversion element according to claim 1, wherein the material is represented by the general formula (1) or (2).

4. The material for a photoelectric conversion element according to claim 1, wherein the group having the aromatic ring structure is represented by the following formula (4a), (4b), (5a), (5b), or (5c): 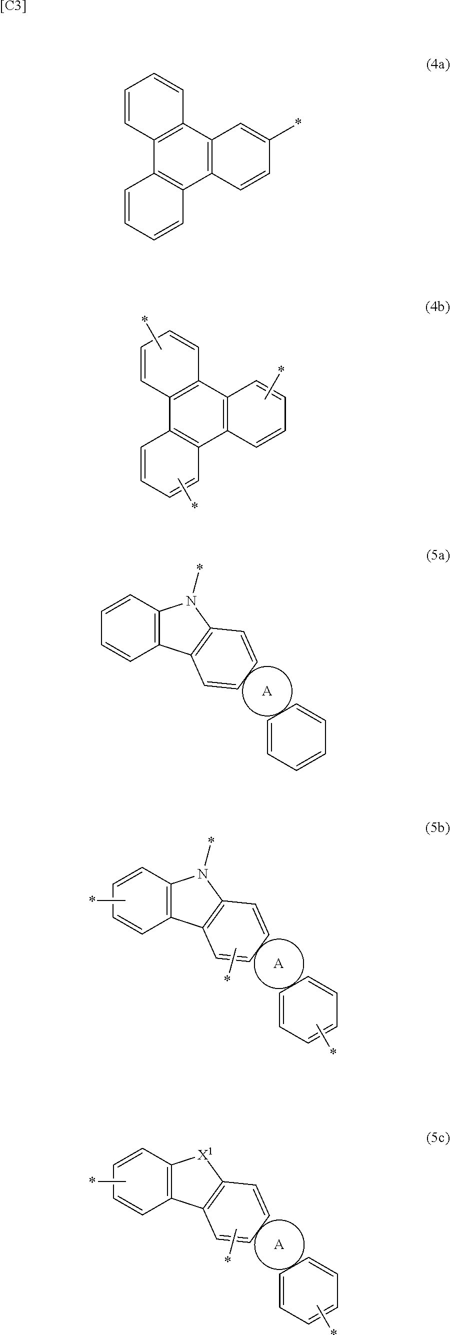
wherein a ring A is same as in formula 5; and “*” represents a bonding point, and at least two thereof are bonding points in formulae (4b), (5b), and (5c).

5. The material for a photoelectric conversion element according to claim 4, wherein the formula (4b) is represented by the following formula (4c): 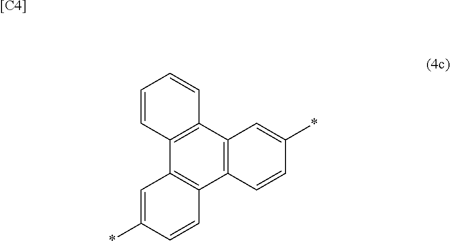
wherein “*” represents a bonding point.

6. The material for a photoelectric conversion element according to claim 1, wherein an energy level of highest occupied molecular orbital (HOMO) obtained by structural optimization calculation with a density functional calculation B3LYP/6-31G(d) is −4.5 eV or lower.

7. The material for a photoelectric conversion element according to claim 1, wherein an energy level of lowest unoccupied molecular orbital (LUMO) obtained by the structural optimization calculation with a density functional calculation B3LYP/6-31G(d) is −2.5 eV or higher.

8. The material for a photoelectric conversion element according to claim 1, wherein the material has a hole mobility of 1×10⁻⁶ cm²/Vs or more.

9. The material for a photoelectric conversion element according to claim 1, wherein the material is amorphous.

10. The material for a photoelectric conversion element according to claim 1, wherein the material is used as a hole transport material of a photoelectric conversion element for imaging.

11. A photoelectric conversion element for imaging, comprising a photoelectric conversion layer and an electron blocking layer between two electrodes, wherein at least one layer of the photoelectric conversion layer and the electron blocking layer contains the material for a photoelectric conversion element according to claim 1.

12. The photoelectric conversion element for imaging according to claim 11, wherein the electron blocking layer contains the material for the photoelectric conversion element.

13. The photoelectric conversion element for imaging according to claim 11, wherein the photoelectric conversion layer contains an electron transport material.

14. The photoelectric conversion element for imaging according to claim 11, wherein the electron blocking layer contains the material for the photoelectric conversion element, and the photoelectric conversion layer contains a fullerene derivative.
